(19) United States
(12) Patent Application Publication
(10) Pub. No.: US 2023/0416344 A1
(43) Pub. Date: Dec. 28, 2023

(54) METHODS FOR TREATING A COMPLEMENT MEDIATED DISORDER CAUSED BY VIRUSES

(71) Applicants: Alexion Pharmaceuticals, Inc., Boston, MA (US); Assistance Publique, Hopitaux de Paris, Paris (FR); Djillali Annane, Maisons-Alfort (FR); Veronique Fremeaux-Bacchi, Vanves (FR); Regis Peffault De Latour, Neuilly-sur-Seine (FR)

(72) Inventors: Sharon Barr, Madison, CT (US); Derek Dunn, Somerville, MA (US); Xiang Gao, Guilford, CT (US); Shamsah D. Kazani, Watertown, MA (US); Michele Mercuri, Boston, MA (US); Jonathan Monteleone, Raleigh, NC (US); Stephan Ortiz, Wellesley, MA (US); Scott T. Rottinghaus, Salem, CT (US); Martine Zimmermann, Saint-Germain-en-Laye (FR); Djillali Annane, Maisons-Alfort (FR); Veronique Fremeaux-Bacchi, Vanves (FR); Regis Peffault De Latour, Neuilly-sur-Seine (FR)

(21) Appl. No.: 17/918,863

(22) PCT Filed: Apr. 16, 2021
(86) PCT No.: PCT/US2021/027636

§ 371 Date: Oct. 13, 2022

Related U.S. Application Data
(60) Provisional application No. 63/010,905, filed on Apr. 16, 2020.
(60) Provisional application No. 63/014,999, filed on Apr. 24, 2020.
(60) Provisional application No. 63/019,050, filed on May 1, 2020.
(60) Provisional application No. 63/020,195, filed on May 5, 2020.
(60) Provisional application No. 63/020,286, filed on May 5, 2020.
(60) Provisional application No. 63/033,140, filed on Jun. 1, 2020.
(60) Provisional application No. 63/063,538, filed on Aug. 10, 2020.

(51) Int. Cl. C07K 16/18; A61K 45/06; A61P 31/14; G01N 33/569

(52) U.S. Cl. CPC C07K 16/18; A61K 45/06; A61P 31/14; G01N 2469/20; C07K 2317/76; C07K 2317/24; G01N 33/56983

(57) ABSTRACT

The present disclosure relates to, inter alia, a method of treating a complement mediated disorder caused by a virus, e.g., corona virus; Dengue virus (DENY); Ross River vims (RRV) and/or influenza virus (flu) by administering an effective amount of a complement modulator, such as, e.g., C5 inhibitor, such as eculizumab or an eculizumab variant or a C5a inhibitor such as olendalizumab (ALXN1007) or a variant thereof, to the subject. In addition, the present disclosure relates to, inter alia, a method of treating human patients inflicted with severe coronavirus disease-2019 (severe COVID-19) who is undergoing treatment with eculizumab. The method includes measuring a level of circulating component C5b-9 (membrane attack complex), in the patient's blood sample to titrate an effective eculizumab dose for the treatment of COVID-19.

### CROSS REFERENCE TO RELATED APPLICATIONS

This application claims the benefit of U.S. Provisional Application No. 63/010,905, filed Apr. 16, 2020, U.S. Provisional Application No. 63/014,999, filed Apr. 24, 2020, U.S. Provisional Application No. 63/019,050, filed May 1, 2020, U.S. Provisional Application No. 63/020,195, filed May 5, 2020, U.S. Provisional Application No. 63/020,286, filed May 5, 2020, U.S. Provisional Application No. 63/033,140, filed Jun. 1, 2020, and U.S. Provisional Application No. 63/063,538, filed Aug. 10, 2020, each of which is incorporated by reference herein in its entirety.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition.

While a properly functioning complement system provides a robust defense against infecting microbes, inappropriate regulation or activation of the complement pathways has been implicated in the pathogenesis of a variety of disorders, including disorders caused by infectious agents.

Non-clinical data support the role of complement 3 (C3) in mediation of lung injury elicited by infectious agents. For instance, in a mouse model of coronavirus (CoV), infection of C57BL/6J mice with mouse-adapted severe acute respiratory syndrome coronavirus (SARS-CoV) results in high-titer virus replication within the lung, induction of inflammatory cytokines and chemokines, and immune cell infiltration within the lung. See, Gralinski et al. (mBio, 2018 Oct. 9; 9(5); PMID: 30301856). Since C3 deposition was evident on day 2 and day 4 post infection with SARS-CoV, the authors hypothesize that it is likely that complement deposition contributes to pulmonary disease and inflammatory cell recruitment in the in vivo mouse model.

Studies with transgenic and/or knockout animal models further point to a role of complement system in the pathogenesis of pulmonary dysfunction following infection with viruses targeting the respiratory system. In mice treated with a mouse-infective coronavirus, infection is attenuated in C3 knockout mice, as evidenced by (a) protection against SARS-CoV-induced weight loss); (b) attenuation in pathological features (e.g., (1) presence of inflammatory cells in the large airway and parenchyma; (2) perivascular cuffing; (3) thickening of the interstitial membrane; and (4) intra-alveolar edema); (c) improved respiratory function; and/or (d) reduction in inflammatory cytokines/chemokines in the lung and its periphery. See, Gralinski et al. (supra). Gralinski further found that C3-deficient mice had reduced neutrophilia in their lungs and reduced systemic inflammation, thereby resulting in attenuation in infection. Gralinski et al. propose that inhibition of C3 complement may be therapeutically effective against coronavirus-mediated disease.

While Gralinski's studies using C3 inhibition in mice models suggest that C3 antagonism protects against SARS-CoV infection, inhibition of complement alternate pathway alone is insufficient. For instance, Factor B (fB) knockout (−/−) and complement 4 (C4) knockout (−/−) mice do not have the same protection from CoV-mediated weight loss as compared to complement 3 (C3) knockout (−/−) mice.

Excessive complement activation has also been postulated to serve an important factor that contributes to acute lung injury after Middle East respiratory syndrome coronavirus (MERS-CoV) infection. This has been demonstrated in vivo using a transgenic mouse model for MERS-CoV. See, Jiang et al. (Emerg Microbes Infect. 2018 Apr. 24; 7(1):77; PMID: 29691378). In this mouse model, MERS-CoV causes severe acute respiratory failure and high mortality accompanied by an elevated secretion of cytokines and chemokines. Histopathological analysis revealed that complement was excessively activated and concomitantly, increased concentrations of the C5a and C5b-9 complement activation products were observed in sera and lung tissues, respectively. Blocking C5aR, using an antibodies, alleviated lung and spleen tissue damage and reduced inflammatory responses. Furthermore, anti-C5aR antibody treatment attenuated viral replication in lung tissues. These results showed that blockade of the C5a-C5aR alleviates lung damage in a transgenic mice model that has been infected with MERS-CoV.

A similar finding has been reported in the context of infections mediated by influenza virus strain H5N1 (commonly called “bird flu”). See, Sun et al. (Am J Respir Cell Mol Biol. 2013 August; 49(2):221-30; PMID: 23526211). Sun showed that acute lung injury (ALI) in H5N1-infected mice was caused by excessive complement activation, as demonstrated by deposition of C3, C5b-9, and mannose-binding lectin C (MBL)-C in lung tissue, and by up-regulation of MBL-associated serine protease-2 and the complement receptors C3aR and C5aR. Treating H5N1-infected mice with a C3aR antagonist led to significantly reduced lung inflammation, alleviating ALI. Additionally, treating H5N1 challenged mice with anti-C5a antibody or depleting complement with cobra venom factor afforded protection that was similar to C3aR antagonist-treated mice. These results show a role of complement in H5N1-induced ALI and that C3aR and/or C5a antagonism may provide either direct or adjunctive options for therapy.

A recent report points to a role of nucleocapsid proteins SARS-CoV, SARS-CoV-2 and MERS-CoV in complement activation via the mannan-binding lectin (MBL) pathway (Gao et al., MedXriv, Posted Mar. 30, 2020; DOI: 10.1101/2020.03.29.20041962). It was shown that N proteins of SARS-CoV, MERS-CoV and SARS-CoV-2 bind to, and thereby potentiate MBL-associated serine protease-2 (MASP-2)-dependent complement activation, which in turn, aggravates LPS-induced pneumonia by MASP-2-involved complement activation in a mouse model. Further immunohistochemical staining of lung tissues of human COVID19 patients showed deposit of MBL, MASP-2, C4alpha, C3 and C5b-9 in type I and type II alveolar epithelia cells, as well as inflammatory cells, some hyperplastic pneumocytes, and exudates in alveolar spaces with necrotic cell debris. Further, significantly increased serum C5a level was also observed in COVID-19 patient, particularly in severe cases. Treatment with a recombinant anti-C5a monoclonal antibody (BDB-001) conferred some clinical benefit to two patients with COVID-19.

While the above references indicate that lung injury following viral infection is mediated, in part, by the members of the complement system such as C3, C5a, C5b-9, including receptors such as C3aR and C5aR, they are silent regarding a role of complement 5 (C5) in the pathogenesis of coronavirus-induced lung injury. Moreover, there is little information, if any, as to what therapeutic roles C5/C5a antagonists, such as eculizumab, ravulizumab, olendalizumab (ALXN 1007), or antigen-binding fragments thereof, including, antibody derivatives such as bispecific minibodies comprising the antigen-binding fragments (e.g., ALXN1720), may play in alleviating lung injury in subjects infected with coronavirus.

There is an immediate and unmet need for developing effective strategies for the prevention, treatment and/or management of subjects, e.g., human patients and veterinary animals, that have been infected with coronavirus.

### SUMMARY

The present disclosure provides a method of treating a complement mediated disorder caused by a virus, e.g., coronavirus such as SARS-CoV, MERS-CoV, or severe acute respiratory syndrome coronavirus 2 (SARS-CoV-2), COVID-19 coronavirus (2019-nCoV); Dengue virus (DENV); Ross River virus (RRV) and/or influenza virus (flu) in a subject comprising administering an effective amount of a modulator of a complement pathway, e.g., classical pathway (CP) alternate pathway (AP), and/or lectin pathway comprising, e.g., mannose-binding lectin (MBL) or ficolin binding to certain sugars.

The present disclosure is based, in part, on the understanding that the complement functions as an immune surveillance system that rapidly responds to viral infection and plays a pivotal role in inflammatory responses triggered in response to infection by one or more of the aforementioned viruses, e.g., coronavirus such as SARS-CoV, MERS-CoV, or SARS-CoV-2 (2019-nCoV); Dengue virus (DENV); Ross River virus (RRV) and/or influenza virus (flu) and that modulation of the complement system, e.g., using inhibitors of the complement pathway provide therapeutic benefit.

In another aspect, the present disclosure is based, in part, on the discovery that circulating complement sC5b9 (membrane attack complex) levels are elevated in human patients suffering from COVID-19, especially, in severe COVID-19 patients needing hospitalization and/or intensive care unit (ICU) stay. The increase in circulating terminal complement components C5b-9, C4d, C3 and C4 in these patients highlights the systemic C5 cleavage in COVID-19. Treatment with a high dose of eculizumab suppressed levels of terminal component in patients with severe COVID-19.

Additionally, a significant correlation was observed between circulating sC5b-9 levels at time of sampling and patient outcome, e.g., time to discharge from hospital. These data point to a contributory role of C5 activation, as indicated by elevated circulating sC5b9 levels, in COVID-19 disease severity. The disclosure further provides a method for treating severe COVID-19 in human patients by administering eculizumab at a dose that attenuates terminal complement sC5b9 levels, e.g., to a baseline level of about 340 ng/ml or lower, thereby reducing the duration of hospitalization in severely ill COVID-19 patients.

In some embodiments, the disclosure provides a method of treating a complement mediated disorder caused by a virus, e.g., coronavirus such as SARS-CoV, MERS-CoV, or SARS-CoV-2 (2019-nCoV); Dengue virus (DENV); Ross River virus (RRV) and/or influenza virus (flu) in a subject comprising administering an effective amount of a modulator of a complement pathway, e.g., classical pathway (CP). Preferably, the disclosure provides a method of treating a complement mediated disorder caused by a coronavirus in a subject comprising administering an effective amount of an inhibitor of one or more members of the CP, e.g., C1r/s or MASP inhibitor such as CINRYZE; BERINERT; OR RUCONEST; or CIs inhibitor such as Sutimlimab or BIVV020 or C1s inhibitor peptide from RaPharma.

In some embodiments, the disclosure provides a method of treating a complement mediated disorder caused by a virus, e.g., coronavirus such as SARS-CoV, MERS-CoV, or SARS-CoV-2 (2019-nCoV); Dengue virus (DENV); Ross River virus (RRV) and/or influenza virus (flu) in a subject comprising administering an effective amount of a modulator of a complement pathway, e.g., alternate pathway (AP). Preferably, the modulator of the AP is an inhibitor of the terminal AP pathway, e.g., an inhibitor of C5/C5a axis or the C3/C3a axis.

In some embodiments, the disclosure provides a method of treating a complement mediated disorder caused by a virus, e.g., coronavirus such as SARS-CoV, MERS-CoV, or SARS-CoV-2 (2019-nCoV); Dengue virus (DENV); Ross River virus (RRV) and/or influenza virus (flu) in a subject comprising administering an effective amount of a modulator of a complement pathway, e.g., lectin pathway (LP). Preferably, the disclosure provides a method of treating a complement mediated disorder caused by a coronavirus in a subject comprising administering an effective amount of an inhibitor of one or more members of the LP, e.g., MASP2 or MASP3 inhibitor such as narsoplimab (MASP2) or OMS906 (MASP3);

The present disclosure provides a method of treating a complement mediated disorder caused by a virus, e.g., coronavirus, such as SARS-CoV, MERS-CoV, or SARS-CoV-2 (2019-nCoV) in a subject comprising administering an effective amount of an inhibitor of complement C5 or C5a protein to the subject. In some embodiments, the human subject with the viral complement-mediated disease exhibits (A) a respiratory symptom selected from: (1)) inflammation of cells in the large airway and parenchyma; (2) perivascular cuffing; (3) thickening of the interstitial membrane; (4) intra-alveolar edema; (5) rhinorrhea; (6) sneezing; (7) sore throat; (8) pneumonia; (9) ground-glass opacity; (10) RNAaemia; and (11) acute respiratory distress syndrome (ARDS); and/or (B) a systemic disorder selected from (1) fever; (2) cough; (3) fatigue; (4) headache; (5) sputum production; (6) haemoptysis; (7) acute cardiac injury; (8) hypoxemia; (9) dyspnoea; (10) lymphopenia; (11) renal injury; and (12) diarrhea.

The present disclosure is based, in part, on the understanding that inhibitors of C5/C5a, e.g., anti-C5 antibodies, such as eculizumab, or anti-C5a antibodies, such as olendalizumab (ALXN1007), are useful for the prevention, amelioration and/or therapy of lung injury elicited in vivo by viral infection, e.g., coronaviral infection caused by SARS-CoV, MERS-CoV, or SARS-CoV-2 (2019-nCoV and/or influenza caused by influenza virus.

In certain aspects, a method is provided of treating a complement mediated disorder caused by a virus that can cause lung or pulmonary injury in a subject (i.e., inflammation of cells in the large airway and parenchyma; (2) perivascular cuffing; (3) thickening of the interstitial membrane; and/or (4) intra-alveolar edema), comprising administering an effective amount of an inhibitor of a complement C5 protein (“a C5 inhibitor”) to the subject. In some embodiments, the virus causing lung or pulmonary injury includes coronavirus such as SARS-CoV, MERS-CoV, or SARS-CoV-2 (2019-nCoV) or flu virus.

In some aspects, the human subject is suffering from severe viral disease comprising shortness of breath (e.g., resting rate>30 breaths/minute; oxygen saturation<93% at rest or arterial oxygen partial pressure (PaO2)/fraction of inspired oxygen (FiO2)<300 mmHg (1 mmHg=0.133 kPa)). In some aspects, the human subject is suffering from critical viral disease comprising respiratory failure requiring mechanical ventilation; respiratory shock; severe pneumonia; acute lung injury (ALI); ARDS requiring oxygen supplementation; and/or combined failure of non-respiratory organs (e.g., heart, kidney) that require ICU monitoring. In some aspects, the human subject is suffering from critical viral disease displays at least one symptom selected from (a) progressive reduction of peripheral blood lymphocytes; (b) progressive increase of peripheral inflammatory cytokines such as IL-6 and C-reactive protein; (c) progressive increase of lactate; and (d) rapid progression of lung pathologies in a short period of time.

In certain aspects, a method is provided of treating lung or pulmonary injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the treatment of lung or pulmonary injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating connective or skeletal tissue injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the virus causing lung or pulmonary injury includes Ross River virus (RRV). In some embodiments, the treatment of connective or skeletal tissue injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating endothelial or vascular injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the virus causing endothelial or vascular injury includes Dengue virus (DENV). In some embodiments, the treatment of endothelial or vascular injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating a subject with coronaviral disease, e.g., 2019-nCoV acute respiratory disease (COVID-19), the method comprising administering to the subject an effective amount of an anti-C5 antibody, or antigen binding fragment thereof, wherein the method comprises an administration cycle comprising an induction phase followed by a maintenance phase, wherein: the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or the anti-C5 antibody, or antigen binding fragment thereof, is administered during the induction phase at a dose of 600 mg weekly for 1 week, starting at day 0, and is administered during the maintenance phase at a dose of 600 mg every week; or the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

In certain aspects, a method for treating a complement mediated disorder caused by a virus (e.g., coronavirus such as SARS-CoV, MERS-CoV, or SARS-CoV-2 (2019-nCoV); Dengue virus (DENV); Ross River virus (RRV) and/or influenza virus (flu)) in a human subject is provided, wherein the method comprises intravenously administering eculizumab at a dose of 900 mg on Days 1, 8, 15, and 22. In one embodiment, the method further comprises administering eculizumab at a dose of 900 mg on Day 4, Day 12, and Day 18.

In some embodiments, the method comprises monitoring complement (e.g., CH50, C3, C4, C4d, sC5b9, C5) and residual eculizumab plasma levels before, during, and after the treatment period. In one embodiment, the method comprises monitoring complement (e.g., CH50, C3, C4, C4d, sC5b9, C5) and residual eculizumab plasma levels before each administration of eculizumab and at Day 1, Day 2, Day 3, and Day 6 to ensure satisfactory drug exposition. In some embodiments, the treatment eliminates the need for intubation (e.g., at day 14). In other embodiments, the treatment results in an improvement on the OMS progression scale compared to baseline. In other embodiments, the treatment results in an improvement on the OMS progression scale at Days 4, 7, and/or 14 compared to baseline. In other embodiments, the treatment results in a decreased time to discharge. In other embodiments, the treatment results in a decreased time to oxygen supply independency. In other embodiments, the treatment results in a decreased time to negative viral excretion. In other embodiments, the treatment results in an improvement in one or more biological parameters (e.g., C5b9, estimated GFR, CRP, myoglobin, CPK, cardiac troponin, ferritin, lactate, cell blood count, liver enzymes, LDH, D-Dimer, albumin, fibrinogen, triglycerides, coagulation tests, urine electrolyte, creatinuria, proteinuria, uricemia, IL6, procalcitonin, immunophenotype and/or exploratory tests).

In other embodiments, the patient requires hospitalization and/or treatment in an intensive care unit (ICU). In some embodiments, the treatment results in a decrease in organ failure at Day 3 (e.g., as defined by the relative variation in Sequential Organ Failure Assessment (SOFA) score at Day 3) in the ICU patient. In other embodiments, the treatment results in a decrease or elimination of secondary infections (e.g., pneumonia acquired) in the ICU patient. In other embodiments, the treatment results in vasopressor free survival (e.g., pneumonia acquired) in the ICU patient. In other embodiments, the treatment results in ventilator free survival in the ICU patient. In other embodiments, the treatment results in a decreased incidence of dialysis in the ICU patient. In other embodiments, the treatment results in an improvement on the OMS progression scale for the ICU patient compared to baseline. In other embodiments, the treatment results in an improvement on the OMS progression scale for the ICU patient compared to baseline at Days 4, 7 and 14 days, overall survival at 14, 28 and 90 days, 28-day ventilator free-days, improved evolution of PaO2/FiO2 ratio, decreased respiratory acidosis at day 4 (arterial blood pH of <7.25 with a partial pressure of arterial carbon dioxide [Paco2] of ≥60 mm Hg for >6 hours), decreased time to oxygen supply independency, decreased duration of hospitalization, decreased time to negative viral excretion, and/or decreased time to ICU and hospital discharge. In other embodiments, the treatment results in an improvement in one or more of the following biological parameters for the ICU patient: sC5b9, estimated GFR, CRP, cardiac troponin, urine electrolyte and creatinine, proteinuria, uricemia, IL6, myoglobin, KIM-1, NGAL, CPK, ferritin, lactate, cell blood count, liver enzymes, LDH, D-Dimer, albumin, fibrinogen, triglycerides, coagulation tests (including activated partial thromboplastin time), procalcitonin, immunophenotype, exploratory tests, rate of renal replacement therapy, and/or ventilation parameters.

In certain aspects, a method for treating a subject with coronaviral disease (e.g., COVID-19) is provided, wherein the method comprises intravenously administering eculizumab at a dose of 1200 mg on Days 1, 4, and 8. In some embodiments, the method comprises intravenously administering eculizumab at a dose of 1200 mg on Days 1, 4, and 8 and 900 mg on Days 15 and 22. In other embodiments, the method comprises intravenously administering eculizumab (a) at a dose of 1200 mg on Days 1, 4, and 8, (b) at a dose of 900 mg on Days 15 and 22, and (c) at a dose of 900 mg or 1200 mg on Days 12 and 18. In some embodiments, eculizumab is administered based on the therapeutic dose monitoring (TDM). In some embodiments, TDM comprises monitoring of a parameter selected from eculizumab plasma level and free C5 free C-5, and/or CH50 suppression, wherein, the optional dose is administered if the parameter is modulated (e.g., attenuated) compared to a reference standard. In some embodiments, the treatment results in improved mechanical ventilation status, improved oxygen saturation levels (SpO2 and/or PaO2), improved supplemental oxygen status, decreased time in the intensive care unit, and/or decreased duration of hospitalization.

In certain aspects, a method is provided for treating a subject with coronaviral disease, e.g., COVID-19, comprising intravenously administering ravulizumab on Day 1 based on weight-based loading dose per label (e.g., United States Product Insert (USPI) label for ULTOMIRIS® (ravulizumab-cwvz) injection, for intravenous use; Initial U.S. Approval: 2018; Revised: 10/2019); intravenously administering 900 mg (or 600 mg for patients<60 kg) on day 5 (D5); intravenously administering 900 mg (or 600 mg for patients<60 kg) of ravulizumab on Day 10 (D10) and intravenously administering 900 mg of ravulizumab for all patients on Day 15 (D15). For example, in one embodiment, ravulizumab is administered to the patient on Day 5 and Day 10 at a dose of 600 mg or 900 mg (based on weight category) and then on Day 15 at a dose of 900 mg. Specifically, a weight-based dose is administered on Day 1 as follows: Patients weighing≥40 to <60 kg: 2400 mg/kg; ≥60 to <100 kg: 2700 mg/kg; or ≥100 kg: 3000 mg/kg on Day 1. On Day 5 and Day 10, doses of 600 mg or 900 mg ravulizumab are administered (according to weight category) and on Day 15 patients receive 900 mg ravulizumab. Final assessment is performed at Day 29 or on day of discharge, whichever occurs first. Screening and the Day 1 visits can occur on the same day if the patient has met all inclusion and no exclusion criteria. In some embodiments, the treatment improves the survival rate of patients with SARS CoV 2 infection who are receiving ravulizumab plus best supportive care (BSC) compared with BSC alone. In other embodiments, the treatment decreases lung injury in patients with SARS CoV 2 infection while on supportive medical care. In other embodiments, the treatment improves clinical outcomes in patients with SARS CoV 2 infection while on supportive medical care. In some embodiments, the treatment results in one ore more of the following: (1) a decrease number of days free of mechanical ventilation at Day 29, (2) decreased duration of intensive care unit stay at Day 29, (3) improved change from baseline in sequential organ failure assessment at Day 29, (4) improved change from baseline in SpO2/FiO2 at Day 29, (5) decreased duration of hospitalization at Day 29, and/or (5) survival (based on all-cause mortality) at Day 60 and Day 90.

In certain aspects, a method of treating a subject with coronaviral disease (e.g., COVID-19) is provided, wherein the method comprises administering eculizumab to the patient according to a uniform schedule of eculizumab (e.g., 4 doses of 1200 mg every 3 days, followed by 3 doses of 900 mg every 3 days) until oxygen support independence. In some embodiments, the patient is an intubated patient (e.g., severe, non-ICU).

In certain aspects, a method of treating severe coronavirus disease-2019 (COVID-19) in a human patient infected with SARS-CoV-2 (2019-nCoV) is provided, wherein the method comprises administering an effective amount a pharmaceutical composition comprising eculizumab (SOLIRIS®). In one embodiment, the severe COVID-19 comprises a need for hospitalization and/or treatment in an intensive care unit (ICU).

In certain aspects, a method of effectively treating severe coronavirus disease-2019 (severe COVID-19) in a human patient with eculizumab is provided, wherein the method comprises: (a) measuring a level of a marker which is C5b-9 (membrane attack complex; MAC) in the patient's blood sample, prior to and after treatment with eculizumab; (b) comparing the marker level to a reference standard; (c) titrating the treatment dose of eculizumab until the marker level in the human patient converges towards the reference standard; and (d) administering the titrated dose of eculizumab to the human patient. In one embodiment, the marker is circulating sC5b9 level and the reference standard comprises a level of about 340 ng/ml, wherein the effective treatment comprises reduction in duration of hospitalization and/or length of intensive care unit (ICU) stay. In another embodiment, the marker comprises circulating sC5b9 level and the reference standard comprises a level of about 340 ng/ml, wherein a positive differential (e.g., sC5b9 levels in the patient's sample>about 340 ng/ml) indicates longer hospitalization and/or ICU stays.

In certain aspects, a method of prognosticating an outcome which is duration of hospitalization and/or treatment in an intensive care unit (ICU) in a human patient inflicted with severe coronavirus disease-2019 (severe COVID-19) is provided, wherein the method comprises measuring a level of a marker which is C5b-9 (membrane attack complex; MAC) in the patient's blood sample, wherein an increase in marker level compared to a reference standard is prognostic of the outcome.

Numerous other aspects are provided in accordance with these and other aspects of the invention. Other features and aspects of the present invention will become more fully apparent from the following detailed description and the appended claims.

### BRIEF DESCRIPTION OF THE DRAWING

FIG. 1 shows individual plasma free eculizumab concentration-time profiles (N=6). Regions in red are considered to have sub-therapeutic eculizumab exposures. Arrows signify planned SOLIRIS 900 mg dosing. The top middle profile is data from a patient who died 6 days after initiation of treatment with SOLIRIS. X-axis: Time (days); Y-axis: Plasma free eculizumab concentrations (μg/mL).

FIG. 2 shows individual plasma soluble C5b9 concentration-time profiles (N=6). Arrows signify second eculizumab 900 mg dosing, administered 7 days after treatment initiation with eculizumab 900 mg. The top middle profile is data from a patient who died 6 days after initiation of treatment with SOLIRIS. X-axis: Time (days); Y-axis: Soluble C5b9 (ng/mL).

FIG. 3 shows individual profiles: CH50 and time-matched soluble C5b9 profiles (N=1). Arrows signify eculizumab 900 mg dosing. Abbreviations and terms: jour=day; sC5b9=soluble C5b9.

FIG. 4 shows a schematic of the study protocol.

FIG. 5 shows eculizumab clearance changes over time in pediatric patients with thrombotic microangiopathy following hematopoietic stem-cell transplant.

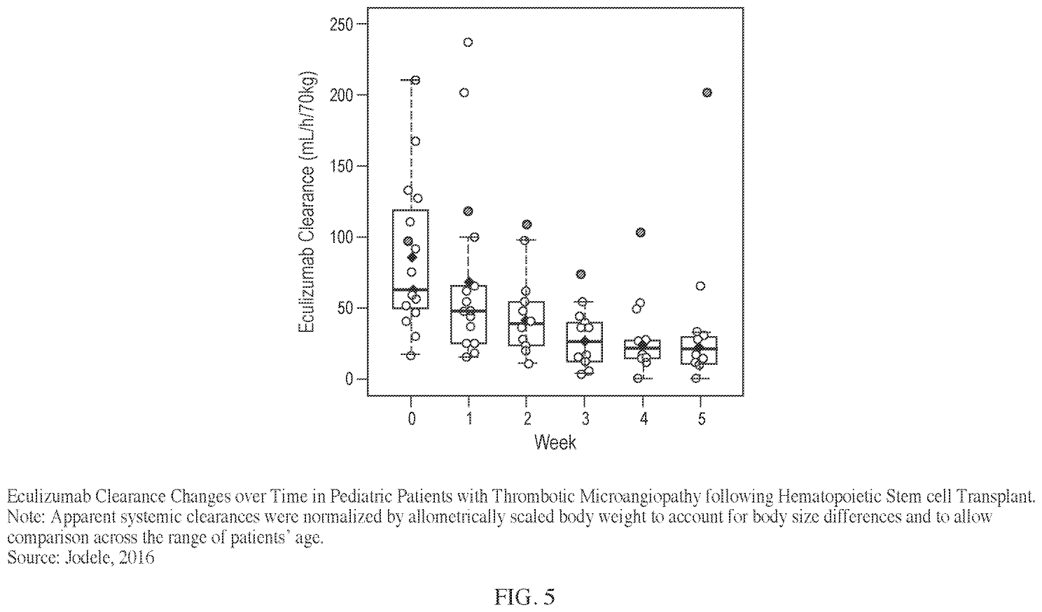

FIG. 6 shows simulations comparing “With” and “Without” the faster clearance assumption of ravulizumab in adult patients with thrombotic microangiopathy following hematopoietic stem-cell transplant.

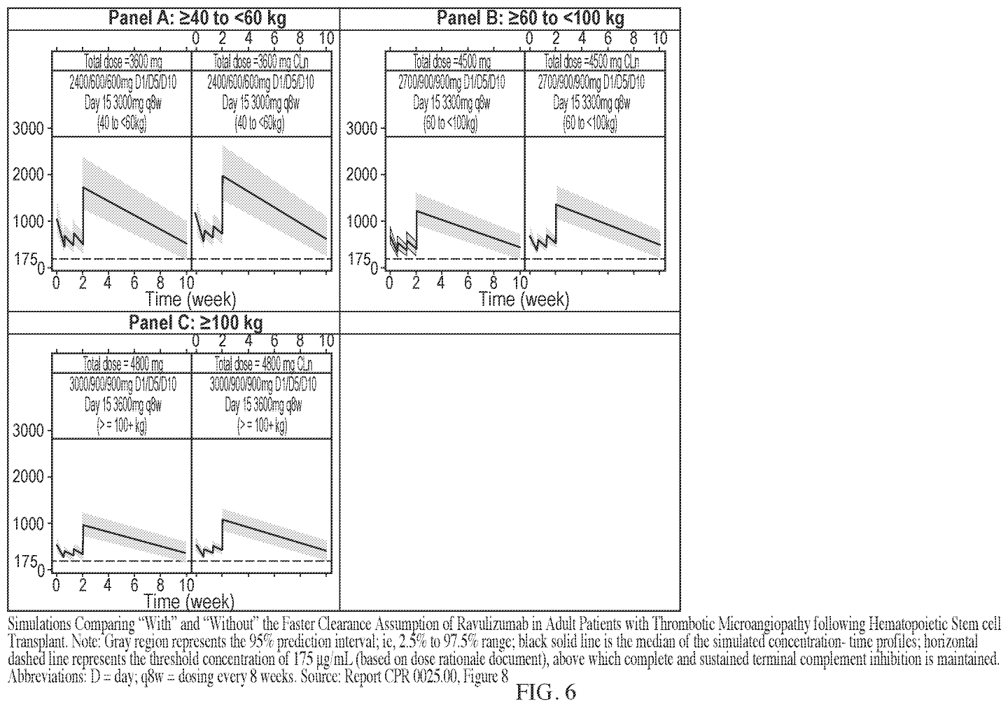

FIG. 7 shows Kaplan-Meier estimated probability of survival.

FIG. 8 shows change from baseline to day 1 and day 7 in (a) CH50 activity in patients treated with and without eculizumab and (b) free residual eculizumab in patients treated with eculizumab. Each diamond represents 1 patient sample.

FIGS. 9A-9B shows data on complement assessment in patients with COVID-19. Specifically, FIG. 9A shows circulating levels of sC5b-9 in healthy controls (left) and patients with COVID-19 (right). Patients with COVID-19 were sampled during their hospitalization. The normal values of sC5b-9 are below 340 ng/ml. FIG. 9B shows a Kaplan Meyer representation of time to discharge according to circulating levels of sC5b-9 at time of sampling. Median delay of blood sampling after admission was 2 days (interquartile range, 1;3).

### DETAILED DESCRIPTION

As used herein, the word “a” or “plurality” before a noun represents one or more of the particular nouns. For example, the phrase “a mammalian cell” represents “one or more mammalian cells.”

The singular form “a,” “an,” and “the” include plural reference unless the context clearly dictates otherwise.

The term “about”, particularly in reference to a given quantity or number, is meant to encompass deviations within plus or minus ten percent (+10%), (e.g., ±5%).

The term “pharmaceutical formulation” refers to preparations which are in such form as to permit the biological activity of the active ingredients to be unequivocally effective, and which contain no additional components which are significantly toxic to the subjects to which the formulation would be administered.

The term “recombinant protein” is known in the art. Briefly, the term “recombinant protein” can refer to a protein that can be manufactured using a cell culture system. The cells in the cell culture system can be derived from, for example, a mammalian cell, including a human cell, an insect cell, a yeast cell, or a bacterial cell. In general, the cells in the cell culture contain an introduced nucleic acid encoding the recombinant protein of interest (which nucleic acid can be borne on a vector, such as a plasmid vector). The nucleic acid encoding the recombinant protein can also contain a heterologous promoter operably linked to a nucleic acid encoding the protein.

The term “mammalian cell” is known in the art and can refer to any cell from or derived from any mammal including, for example, a human, a hamster, a mouse, a green monkey, a rat, a pig, a cow, a hamster, or a rabbit. In some embodiments, the mammalian cell can be an immortalized cell, a differentiated cell, or an undifferentiated cell.

The term “immunoglobulin” is known in the art. Briefly, the term “immunoglobulin” can refer to a polypeptide containing an amino acid sequence of at least 15 amino acids (e.g., at least 20, 30, 40, 50, 60, 70, 80, 90, or 100 amino acids, or more than 100 amino acids) of an immunoglobulin protein (e.g., a variable domain sequence, a framework sequence, or a constant domain sequence). The immunoglobulin can, for example, include at least 15 amino acids of a light chain immunoglobulin, e.g., at least 15 amino acids of a heavy chain immunoglobulin, such as a CDRH3. The immunoglobulin may be an isolated antibody (e.g., an IgG, IgE, IgD, IgA, or IgM). The immunoglobulin may be a subclass of IgG (e.g., IgG1, IgG2, IgG3, or IgG4). The immunoglobulin can be an antibody fragment, e.g., a Fab fragment, a F(ab′)₂ fragment, or a scFv.

The immunoglobulin can also be an engineered protein containing at least one immunoglobulin domain (e.g., a fusion protein). The engineered protein or immunoglobulin-like protein can also be a bi-specific antibody or a tri-specific antibody, or a dimer, trimer, or multimer antibody, or a diabody, a DVD-Ig, a CODV-Ig, an AFFIBODY®, or a NANOBODY®. Non-limiting examples of immunoglobulins are described herein, and additional examples of immunoglobulins are known in the art.

The term “engineered protein” is known in the art. Briefly, the term “engineered protein” can refer to a polypeptide that is not naturally encoded by an endogenous nucleic acid present within an organism (e.g., a mammal). Examples of engineered proteins include modified enzymes with one or more amino acid substitutions, deletions, insertions, or additions that result in an increase in stability and/or catalytic activity of the engineered enzyme, fusion proteins, humanized antibodies, chimeric antibodies, divalent antibodies, trivalent antibodies, four binding domain antibodies, a diabody, and antigen-binding proteins that contain at least one recombinant scaffolding sequence.

The terms “polypeptide,” “peptide,” and “protein” are used interchangeably and are known in the art and can mean any peptide-bond linked chain of amino acids, regardless of length or post-translational modification.

The term “antibody” is known in the art. The term “antibody” is sometimes used interchangeably with the term “immunoglobulin.” Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a DVD-Ig, a CODV-Ig, an AFFIBODY®, or a NANOBODY®.

The term “antigen-binding fragment” or similar terms are known in the art and can, for example, refer to a fragment of an antibody that retains the ability to bind to a target antigen (e.g., human C5) and inhibit the activity of the target antigen. Such fragments include, e.g., a single chain antibody, a single chain Fv fragment (scFv), an Fd fragment, a Fab fragment, a Fab′ fragment, or an F(ab′)2 fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., C5, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, unless otherwise indicated, an antibody that “specifically binds to human C” refers to an antibody that binds to soluble or cell bound human C5 with a K_{D} of 10⁻⁷ M or less, such as approximately less than 10⁻⁸ M, 10⁻⁹ M or 10⁻¹⁰ M or even lower.

The term “kₐ” is well known in the art and can refer to the rate constant for association of an antibody to an antigen. The term “k_{d}” is also well known in the art and can refer to the rate constant for dissociation of an antibody from the antibody/antigen complex. And the term “K_{D}” is known in the art and can refer to the equilibrium dissociation constant of an antibody-antigen interaction. The equilibrium dissociation constant is deduced from the ratio of the kinetic rate constants, K_{D}=kₐ/k_{d}. Such determinations are typically measured at, for example, 25° C. or 37° C. For example, the kinetics of antibody binding to human C5 can be determined at pH 8.0, 7.4, 7.0, 6.5 and 6.0 via surface plasmon resonance (“SPR”) on a BIAcore 3000 instrument using an anti-Fc capture method to immobilize the antibody.

As used herein, the term “preventing” is art-recognized, and when used in relation to a condition, such as a local recurrence, a disease such as coronavirus-mediated lung disorder or a symptom related thereto (e.g., ARDS), is well understood in the art, and includes administration of a composition which reduces the frequency of, or delays the onset of, symptoms of a medical condition in an individual relative to an individual who does not receive the composition.

As used herein, the term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). Preferably, it is intended that the severity of the subject's condition (e.g., lung dysfunction) is reduced or at least partially improved or modified and that some alleviation, mitigation, reversal or decrease in at least one clinical symptom (e.g., weight loss in subjects compared to normal subjects) is achieved.

As used herein, the terms “induction” and “induction phase” are used interchangeably and refer to the first phase of treatment in a clinical trial.

As used herein, the terms “maintenance” and “maintenance phase” are used interchangeably and refer to the second phase of treatment in a clinical trial. In certain embodiments, treatment is continued as long as clinical benefit is observed or until unmanageable toxicity or disease progression occurs.

As used herein, the term “subject” and includes both human subjects and non-human subjects (e.g., veterinary animal or wild animal). Preferably, “subjects” include human patients. As used herein, “effective treatment” refers to treatment producing a beneficial effect, e.g., amelioration of at least one symptom of a disease or disorder in a subject. A beneficial effect can take the form of an improvement over baseline, i.e., an improvement over a measurement or observation made prior to initiation of therapy according to the method.

In certain embodiments, for treating a subject with a viral disease such as influenza, Dengue fever, Ross river fever, coronavirus infection, e.g., a patient suffering from COVID-19, MERS, SARS, or a disease related thereto, effective treatment may refer to alleviation of at least one symptom of the disease.

In certain embodiments, effective treatment may refer to that improves the subject's chance of survival. In certain embodiments, a disclosed method improves the life expectancy of a subject by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease in a subject, or any other desired alteration of a biological system. An effective amount can be administered in one or more administrations. In some embodiments, an “effective amount” is the amount of a C5 inhibitor, such as an anti-C5 antibody, or antigen binding fragment thereof, that improves a pathological outcome, e.g., lung injury and/or inflammation. In some embodiments, In some embodiments, an “effective amount” is the amount of a C5 inhibitor, such as an anti-C5 antibody, or antigen binding fragment thereof, that improves a clinical outcome, e.g., survival of a subject by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

1. The Complement System

As is well known, the complement system acts in conjunction with other immunological systems of the body to defend against intrusion of cellular and viral pathogens. There are at least 25 complement proteins. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade can progress via the classical pathway (“CP”), the lectin pathway, or the alternative pathway (“AP”). The lectin pathway is typically initiated with binding of mannose-binding lectin (“MBL”) to high mannose substrates. The AP can be antibody-independent and can be initiated by certain molecules on pathogen surfaces. The CP is typically initiated by antibody recognition of, and binding to, an antigenic site on a target cell. These pathways converge at the C3 convertase—the point where complement component C3 is cleaved by an active protease to yield C3a and C3b.

The disclosure relates to use of modulators of complement proteins in the therapy of coronaviral diseases and/or symptoms related thereto. As is known in the art, complement proteins in mammals are largely generated in the hepatic tissue and make up about 5% of the plasma protein. The entire cascade progresses from C1 activation through to membrane attack complex (MAC) formation, interactions dictated by de novo binding sites revealed following protein conformational changes resulting from proteolytic cleavage of the circulating, native protein (C3, C4, C2, FB, C5) or as a consequence of unfolding (C9) or protein/protein interaction (C6-C9).

In some embodiments, the disclosure relates to a use of modulators of the classical pathway (CP) in the therapy of coronaviral diseases. Generally, CP is initiated when antigen-antibody complexes bind the recognition moiety, C1q, triggering activation of the associated proteases, C1r and C1s. Activated C1s cleaves C4 to C4b which binds covalently through its thioester to the target and there captures C2 which is also cleaved by C1s to form the CP C3 convertase C4b2a.

In some embodiments, the disclosure relates to therapy of coronaviral diseases using modulators of the lectin pathway (LP). Generally, LP differs from the CP only in the recognition/initiation unit which binds to bacterial sugars, lectins such as mannose binding lectin (MBL), ficolins or collectins. All bind carbohydrate epitopes, triggering activation of the associated proteases MASP1 and MASP2, the latter cleaving C4 and C2 to form C4b2a. C4b2a cleaves C3 to C3b, exposing the internal thioester that covalently binds C3b to surfaces, causing activating surfaces to become densely coated in C3b (opsonised), providing ligands for phagocyte uptake of the target, a crucial defense against infection. C3b also associates with the C3 convertase to create the C5 convertase C4b2a3b.

In some embodiments, the disclosure relates to therapy of coronaviral diseases using modulators of the alternative pathway (AP). Generally, AP is initiated by C3b (generated from the activation pathways or non-specific sources) binding factor B (FB), which is then cleaved by factor D (FD) to form the C3 convertase, C3bBb. C3bBb cleaves C3 to C3b, coating adjacent surfaces and generating a C5 convertase, C3bBbC3b. Activation of C3 in the fluid phase primes the system for rapid amplification on activating surfaces, typified by absence of the regulatory proteins that suppress activation on “self” cells. FB can bind to any C3b deposited on an activating surface, including that resulting from activation of the classical and lectin pathways. Thus, the alternative pathway is known as the amplification loop of the complement cascade and plays a crucial role in amplifying any small trigger to a large downstream response.

Preferably, the disclosure relates to therapy of coronaviral diseases using modulators of the terminal pathway (TAP). Generally, TAP begins with the capture and cleavage of C5 by either of the C5 convertases, releasing a proinflammatory peptide, C5a. C5b remains attached to the convertase and binds sequentially C6 and C7 and, after release of C5b67 from the convertase and association with membrane, C8 and C9 bind to form the lytic MAC. Recent studies have illustrated the structural complexity of the MAC pore. Notably, while MAC efficiently lyses aged (or unprotected) erythrocytes and susceptible bacteria, when formed on nucleated self-cells it triggers a plethora of activation events, many of which are highly pro-inflammatory.

In some embodiments, the disclosure relates to therapy of coronaviral diseases using complement regulatory proteins. These include, for example, plasma proteins factor H (FH) and C4b-binding protein (C4bp) and membrane proteins, CD35, CD46 and CD55 that inhibit the C3/C5 convertases. Control of the enzymes is brought about by decay accelerating activity, characterized by binding of control proteins, such as FH or CD55, to the multimolecular convertases and rapid dissociation of the enzymatic subunit, Bb or C2a. The C3b or C4b that remains is subject to cofactor activity where regulatory proteins bind the remaining subunit, enabling a complement serine protease, factor I (FI) to cleave and inactivate the substrate forming iC3b/C3dg or C4d/C4c. The MAC inhibitor CD59 blocks formation of the lytic pore as soon as C8 is bound to the complex, thus preventing polymerization of C9. Together these control proteins control complement activation on self-tissues.

In some embodiments, the disclosure further relates to modulators of complement receptors (CRs) in the therapy of coronaviral diseases. CRs bind the degradation fragments of C3 and C4, providing an additional route for immune defense. The activation fragments C3a and C5a, bind receptors (C3aR/C5aR1/C5aR2) on numerous cell types to trigger diverse responses, ranging from neutrophil recruitment and activation, to priming of endothelial cells to enhance adhesion. C5a/C5aR interactions activate the NLRP3 inflammasome, impact T cell responses in adaptive immunity and play a multitude of other roles. The receptors CR3 and CR4 on phagocytic cells bind iC3b to promote uptake and clearance of opsonized targets, while C3dg engages CR2 on B cells and follicular dendritic cells (FDCs) to amplify the immune response to opsonized antigens.

Without being bound to a particular theory, the details of the alternative pathway, including role(s) of various components thereof, are provided below.

The AP C3 convertase is initiated by the spontaneous hydrolysis of complement component C3, which is abundant in the plasma in the blood. This process, also known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). This formation of C3(H₂O) allows for the binding of plasma protein Factor B, which in turn allows Factor D to cleave Factor B into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to thus form the surface-bound AP C3 convertase complex containing C3b,Bb. See, e.g., Müller-Eberhard (1988) Ann Rev Biochem 57:321-347.

The AP C5 convertase—(C3b)₂,Bb—is formed upon addition of a second C3b monomer to the AP C3 convertase. See, e.g., Medicus et al. (1976) J Exp Med 144:1076-1093 and Fearon et al. (1975) J Exp Med 142:856-863. The role of the second C3b molecule is to bind C5 and present it for cleavage by Bb. See, e.g., Isenman et al. (1980) J Immunol 124:326-331. The AP C3 and C5 convertases are stabilized by the addition of the trimeric protein properdin as described in, e.g., Medicus et al. (1976), supra. However, properdin binding is not required to form a functioning alternative pathway C3 or C5 convertase. See, e.g., Schreiber et al. (1978) Proc Natl Acad Sci USA 75: 3948-3952, and Sissons et al. (1980) Proc Natl Acad Sci USA 77: 559-562.

The CP C3 convertase is formed upon interaction of complement component C1, which is a complex of C1q, C1r, and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to thereby generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase—C4b,C2a,C3b—is formed upon addition of a C3b monomer to the CP C3 convertase. See, e.g., Müller-Eberhard (1988), supra and Cooper et al. (1970) J Exp Med 132:775-793.

In addition to its role in C3 and C5 convertases, C3b also functions as an opsonin through its interaction with complement receptors present on the surfaces of antigen-presenting cells such as macrophages and dendritic cells. The opsonic function of C3b is generally considered to be one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

The AP and CP C5 convertases cleave C5, which is a 190 kDa beta globulin found in normal human serum at approximately 75 μg/ml (0.4 μM). C5 is glycosylated, with about 1.5-3 percent of its mass attributed to carbohydrate. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol. 146:362-368). The cDNA sequence of the transcript of this human gene predicts a secreted pro-C5 precursor of 1658 amino acids along with an 18 amino acid leader sequence. See, e.g., U.S. Pat. No. 6,355,245.

The pro-C5 precursor is cleaved after amino acids 655 and 659, to yield the beta chain as an amino terminal fragment (amino acid residues +1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i.e., amino acid residues 660-733 of the above sequence). Approximately 20 percent of the 11 kDa mass of C5a is attributed to carbohydrate. The cleavage site for convertase action is at, or immediately adjacent to, amino acid residue 733. A compound that would bind at, or adjacent to, this cleavage site would have the potential to block access of the C5 convertase enzymes to the cleavage site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. A compound, in a mechanism of action consistent with that of the tick saliva complement inhibitor, Ornithodoros moubata C inhibitor (‘OmCI”) (which can be a C5 inhibitor that can be used in the methods of this invention), may also prevent C5 cleavage by reducing flexibility of the C345C domain of the alpha chain of C5, which reduces access of the C5 convertase to the cleavage site of C5. See, e.g., Fredslund et al. (2008) Nat Immunol 9(7):753-760.

C5 can also be activated by means other than C5 convertase activity. Limited trypsin digestion (see, e.g., Minta and Man (1997) J Immunol 119:1597-1602 and Wetsel and Kolb (1982) J Immunol 128:2209-2216) and acid treatment (Yamamoto and Gewurz (1978) J Immunol 120:2008 and Damerau et al. (1989) Molec Immunol 26:1133-1142) can also cleave C5 and produce active C5b.

Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

The first step in the formation of the terminal complement complex involves the combination of C5b with C6, C7, and C8 to form the C5b-8 complex at the surface of the target cell. Upon the binding of the C5b-8 complex with several C9 molecules, the membrane attack complex (“MAC”, C5b-9, terminal complement complex—“TCC”) is formed. When sufficient numbers of MACs insert into target cell membranes the openings they create (MAC pores) mediate rapid osmotic lysis of the target cells, such as red blood cells. Lower, non-lytic concentrations of MACs can produce other effects. In particular, membrane insertion of small numbers of the C5b-9 complexes into endothelial cells and platelets can cause deleterious cell activation. In some cases, activation may precede cell lysis.

C3a and C5a are anaphylatoxins. These activated complement components can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

C5a receptors are found on the surfaces of bronchial and alveolar epithelial cells and bronchial smooth muscle cells. C5a receptors have also been found on eosinophils, mast cells, monocytes, neutrophils, and activated lymphocytes.

While a properly functioning complement system provides a robust defense against infecting microbes, inappropriate regulation or activation of complement has been implicated in the pathogenesis of a variety of disorders, including, e.g., rheumatoid arthritis; lupus nephritis; asthma; ischemia-reperfusion injury; atypical hemolytic uremic syndrome (“aHUS”); dense deposit disease; paroxysmal nocturnal hemoglobinuria (PNH); macular degeneration (e.g., age-related macular degeneration; hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis. See, e.g., Holers et al. (2008) Immunological Reviews 223:300-316.

2. Treatment of Viral Diseases

In some embodiments, the compositions containing modulators of complement pathway, such as, e.g., molecules of Table 1, are useful in the treatment of diseases elicited by viruses which stimulate complement activation in their host subjects, e.g., influenza, Dengue fever, Ross River fever, SARS, MERS, COVID-19, or disease related thereto. More specifically, because terminal complement proteins have been found to be involved in complement-mediated tissue damage triggered by viral infection, inhibitors of C5/C5a, as provided in Table 1, e.g., anti-C5 antibodies (such as eculizumab or ravulizumab or antigen-binding fragments thereof) or anti-C5a antibodies (such as olendalizumab (ALXN1007) or antigen-binding fragments thereof), are particularly useful in the therapy of viral diseases or symptoms related thereto.

In some embodiments, the compositions containing modulators of complement pathway, such as, e.g., molecules of Table 1, are useful in amelioration of symptoms or effects of viral infection. In the context of COVID-19, clinical manifestations in patients show damage to vital organs such as lungs, hearts, and kidneys. Aberrant complement activation and the concomitant aggravated inflammatory lung injury has been observed in COVID-19 patients (Gao et al., supra). Elevated cytokine release has also been observed in COVID-19 patients, which is postulated to play a role in organ failure. Particularly, clinical and experimental models of COVID-19 suggest that the abnormal presence of complement components in the tubular lumen of kidneys leads to the assembly of the complement C5b-9 on the apical brush border of tubular epithelial cells (TECs), and that this is an important factor in the pathogenesis of tubulointerstitial damage (Diao et al., medRxiv, DOI: 10.1101/2020.03.04.20031120, Apr. 10, 2020). Further, in patients with severe or fatal COVID-19, there is also evidence of end organ damage with acute cardiac injury and primarily mildly elevated troponin. Cardiac dysfunction is thought to be mediated via elevated D-dimer, elevated lactate dehydrogenase, elevated total bilirubin, and decreased platelets (Campbell et al., Circulation, 2020 Jun. 2; 141(22):1739-1741). Death among COVID-19 patients was significantly correlated to cardiac injury, as indicated by elevated troponin levels (average troponin I of 0.19 μg/L) (51.2% vs 4.5%, respectively).

Embodiments of the present disclosure relate to use of modulators of complement pathway in preventing organ damage elicited by viral infection. Specifically, since terminal complement inhibitors such as anti-C5 antibodies (e.g., eculizumab or ravilizumab) attenuate both C5a generation and C5b9 depositions (Volokhina et al., Blood. 2015 Jul. 9; 126(2): 278-279), the present disclosure provides use of complement modulators, such as molecules of Table A, in reducing inflammatory response and severe organ damage in patients. Particularly, the molecules of Table 1 are useful in preventing damage to lungs, heart, and kidneys in COVID-19 patients, which may be mediated via elevated levels of C5a anaphylatoxin and C5b9.

3. Coronaviral Diseases

The disclosure relates to treatment of coronaviral disease in a subject comprising administering an effective amount of a modulator of complement pathway, e.g., at least one modulator of Table 1. Preferably, the compositions containing C5 inhibitors, e.g., anti-C5 antibodies such as eculizumab or antigen-binding fragments thereof, are useful in the treatment of diseases elicited by coronaviruses such as SARS coronavirus (SARS-CoV), MERS coronavirus (MERS-CoV), COVID-19 coronavirus (2019-nCoV) or a coronavirus related thereto.

Coronaviruses are enveloped viruses having a capsid having a helical symmetry. They have a positive-sense single-stranded RNA genome and can infect the cells of birds and mammals. The viruses belonging to this very large family are known to be causative agents of colds (for example the hCoV and OC43 viruses), bronchiolitis (for example the NL63 virus) or even certain forms of severe pneumonia such as those observed during SARS epidemic (such as SARS-CoV).

Despite their belonging to the same viral family, important differences exist between the different coronaviruses, both at the genetic and structural level, but also in terms of biology and sensitivity to antiviral molecules. See, e.g., Dijkman et al. (J Formos Med Assoc. 2009 April; 108(4):270-9; PMID: 19369173); de Wit et al. (Nat Rev Microbiol. 2016 August; 14(8):523-34; PMID: 27344959).

a. SARS-CoV

SARS-CoV is a species of coronavirus known to infect certain mammals such as humans. Two strains of the virus have caused outbreaks of severe respiratory diseases in humans: SARS-CoV, which caused an outbreak of severe acute respiratory syndrome (SARS) between 2002 and 2004, and SARS-CoV-2, which since late 2019 has caused an outbreak of coronavirus disease 2019 (COVID-19). Both strains descended from a single ancestor but made the cross-species jump into humans separately. It is thought that SARS-CoV-2 is not a direct descendant of SARS-CoV (Gorbalenya et al., Feb. 11, 2020; world-wide-web at biorxiv(dot)org/content/10.1101/2020.02.07.937862v1). There are hundreds of other strains of SARS-CoV, most of which are only known to infect non-human species: bats are a major reservoir of many strains of SARS-like coronaviruses, and several strains have been identified in palm civets which were likely ancestors of SARS-CoV.

An epidemic of SARS affected 26 countries and resulted in more than 8000 cases in 2003 (WHO Report, 2020). Since then, a small number of cases have occurred as a result of laboratory accidents or, possibly, through animal-to-human transmission. Symptoms of SARS are influenza-like and include fever, malaise, myalgia, headache, diarrhea, and shivering (rigors). No individual symptom or cluster of symptoms has proved to be specific for a diagnosis of SARS. Although fever is the most frequently reported symptom, it is sometimes absent on initial measurement, especially in elderly and immunosuppressed patients. Cough (initially dry), shortness of breath, and diarrhea are present in the first and/or second week of illness. Severe cases often evolve rapidly, progressing to respiratory distress and requiring intensive care.

SARS is transmitted by aerosols of respiratory secretions, by the fecal-oral route, and by mechanical transmission. Most virus growth occurs in epithelial cells. Occasionally the liver, kidneys, heart or eyes may be infected, as well as other cell types such as macrophages. Transmission of SARS-CoV is primarily from person to person. It appears to have occurred mainly during the second week of illness, which corresponds to the peak of virus excretion in respiratory secretions and stool, and when cases with severe disease start to deteriorate clinically. Most cases of human-to-human transmission occurred in the health care setting, in the absence of adequate infection control precautions. Implementation of appropriate infection control practices brought the global outbreak to an end.

In cold-type respiratory infections, growth appears to be localized to the epithelium of the upper respiratory tract. Clinically, most infections cause a mild, self-limited disease (classical “cold” or upset stomach), but there may be rare neurological complications. The disease results in death in about 3 to 10% of cases.

Laboratory diagnosis of SARS can be carried out using ELISA, complement fixation or hemagglutination tests. Growth in culture is usually ineffective for coronavirus isolation. Since the complete genome of SARS-CoV (as well as common variants thereof) have been identified, genetic testing may be used for diagnosis. The genome of SARS-CoV is a 29,727-nucleotide polyadenylated RNA, has 11 open reading frames, and 41% of the residues are G or C. The genomic organization is typical of coronaviruses, with the characteristic gene order (5′-replicase (rep), spike (S), envelope (E), membrane (M), nucleocapsid (N)-3′ and short untranslated regions at both termini. The SARS-CoV rep gene, which comprises about two-thirds of the genome, is predicted to encode two polyproteins that undergo co-translational proteolytic processing. There are four open reading frames (ORFs) downstream of rep that are predicted to encode the structural proteins, S, E, M and N, which are common to all known coronaviruses. The hemagglutinin-esterase gene, which is present between ORFlb and S in group 2 and some group 3 coronaviruses was not found. Phylogenetic analyses and sequence comparisons showed that SARS-CoV is not closely related to any of the previously characterized coronaviruses. Other techniques for detection of bioagents include high-resolution mass spectrometry (MS), low-resolution MS, fluorescence, radioiodination, DNA chips and antibody techniques.

b. MERS-CoV

The MERS-CoV is a new emerging virus identified in 2012 in Saudi Arabia, responsible for SARS and kidney failure. Since its identification, this virus has been responsible for more than 1,806 cases of infection in 26 countries, mainly in the Middle East. It is responsible for 643 deaths or nearly 35.6% mortality according to the World Health Organization (Source WHO, Sep. 28, 2016).

The MERS-CoV belongs to the order of Nidovirales, to the family of Coronaviridae, and to the genus Betacoronavirus. Although most cases of MERS-CoV in humans are attributable to human-to-human transmission, the camel appears to be a permanent intermediate infected animal host of MERS-CoV and thus constitutes the main animal source of infection in humans.

There is currently no prophylactic or therapeutic solution to effectively treat this epidemic respiratory viral pathogen with pandemic potential. Several therapeutic avenues have recently been explored: use of ribavirin, interferon, or mycophenolic acid. Unfortunately, most of these compounds have not shown enough efficacy when used in infected patients (Al-Tawfiq et al., Int J Infect Dis. 2014 March; 20:42-6; PMID: 24406736) or as part of prophylactic treatment (de Wit et al., 2016, supra).

A first strategy for therapy against MERS-CoV was to test, among the many known antiviral molecules, those used to combat SARS-CoV. Thus, inhibitors of viral replication, such as protease inhibitors, helicase inhibitors, and inhibitors of entry of the virus into the target cells were tested in vitro. Dyall et al. (Antimicrob Agents Chemother. 2014 August; 58(8):4885-93; PMID: 24841273) tested different categories of drugs with the aim of identifying anti-viral agents active on the SARS and/or MERS-COV coronaviruses. Among the different classes of agents tested, it was shown that certain anti-inflammatory agents inhibited the proliferation of SARS-CoV, while MERS-CoV was rather inhibited by certain inhibitors of ion transport, inhibitors of tubulin, or apoptosis inhibitors. Out of 290 compounds tested, only 33 compounds with antiviral activity on MERS-CoV were identified in cell culture.

However, owing partly to differences in terms of both protein composition and functional interactions with the host cell, many antiviral compounds effective on SARS-CoV are not systematically active against MERS-CoV, and vice versa. Also, currently, there are no, or very few, therapeutic molecules recognized and/or approved by health authorities to fight infections with the MERS-CoV virus. In addition, there is no vaccine on the market against the MERS-CoV virus. Some candidates are being evaluated in a phase I clinical trial, with ongoing efficacy evaluations (National Clinical Trials #NCT02670187). See, Modjarrad et al. (Lancet Infect Dis. 2019 September; 19(9):1013-1022; PMID: 31351922).

c. Effect of Coronavirus in the Respiratory System

SARS-CoV infection in humans results in an acute respiratory illness that varied from mild febrile illness to ALI and in some cases ARDS and death. See, Channappanavar et al. (Semin Immunopathol. (Review) 2017 July; 39(5):529-539; PMID: 28466096). The clinical course of SARS presents in three distinct phases—(a) an initial phase characterized by robust virus replication accompanied by fever, cough, and other symptoms, all of which subsided in a few days; (b) a second clinical phase associated with high fever, hypoxemia, and progression to pneumonia-like symptoms, with declining virus titers towards the end of this phase; and (c) a third phase in which patients progress to ARDS, often resulting in death. The third phase is thought to have resulted from exuberant host inflammatory responses.

The most common clinical manifestations of MERS include flu-like symptoms such as fever, sore throat, non-productive cough, myalgia, shortness of breath, and dyspnea, which rapidly progress to pneumonia. See, Channappanavar et al. (supra). Other atypical presentations include mild respiratory illness without fever, chills, wheezing, and palpitations. MERS-CoV in humans also causes gastrointestinal symptoms such as abdominal pain, vomiting, and diarrhea. Most MERS patients with dyspnea progress to develop severe pneumonia and require admission to an intensive care unit (ICU). Although most healthy individuals present with mild-moderate respiratory illness, immunocompromised and individuals with comorbid conditions experience severe respiratory illness, which often progressed to ARD. Overall, MERS-CoV caused severe disease in primary index cases, immunocompromised individuals and in patients with comorbid conditions, but secondary cases of household contacts or healthcare workers were mostly asymptomatic or showed mild respiratory illness.

Typically, analyses of lungs from patients who succumbed to SARS showed lung consolidation and edema with pleural effusions, focal hemorrhages, and mucopurulent material in the tracheobronchial tree. Diffuse alveolar damage (DAD) was a prominent histological feature in SARS lungs. Other changes included hyaline membrane formation, alveolar hemorrhage, and fibrin exudation in alveolar spaces with septal and alveolar fibrosis observed during later stages. Staining for viral antigen revealed infection of airway and alveolar epithelial cells, vascular endothelial cells, and macrophages. Furthermore, SARS-CoV viral particles and viral genome were also detected in monocytes and lymphocytes. See, Gu et al. (J Exp Med. 2005 Aug. 1; 202(3):415-24); Nicholls et al. (Lancet. 2003 May 24; 361(9371):1773-8). In addition to these changes, histological examination of lungs from patients who died of SARS revealed extensive cellular infiltrates in the interstitium and alveoli. These cellular infiltrates included neutrophils and macrophages with macrophages being the predominant cell type. These results correlated with increased numbers of neutrophils and monocytes and lower CD4 and CD8 T cell counts in the peripheral blood samples of patients with fatal SARS.

With respect to MERS, analysis of lung tissue from human patient showed pleural, pericardial, and abdominal effusions associated with generalized congestion, edema, and consolidation of lungs (Ng et al., Am J Pathol. 2016 March; 186(3):652-8). Similar to SARS-CoV infection, DAD was a prominent feature in the lungs. Additionally, epithelial cell necrosis, sloughing of bronchiolar epithelium, alveolar edema, and thickening of alveolar septa were also noted. Immunohistochemical examination showed that MERS-CoV predominantly infected airways and alveolar epithelial cells, and endothelial cells and macrophages. The severity of lung lesions correlated with extensive infiltration of neutrophils and macrophages in the lungs and higher numbers of these cells in the peripheral blood of MERS patients.

With respect to etiological agents contributing to lung injury in patients afflicted with pathogenic coronaviruses such as SARS and MERS, cytokines and chemokines have long been thought to play an important role in immunity and immunopathology during virus infections. A rapid and well-coordinated innate immune response is the first line of defense against viral infections, but dysregulated and excessive immune responses may cause immunopathology (Channappanavar et al. (supra)). Although there is no direct evidence for the involvement of pro-inflammatory cytokines and chemokines in lung pathology during SARS and MERS, correlative evidence from patients with severe disease suggests a role for hyper-inflammatory responses in hCoV pathogenesis.

The risk of damage in the pulmonary system due to new types of coronaviral infection is grave. Notably, metagenomics and synthetic virus recovery strategies have since revealed the existence of large pools of pre-epidemic SARS-like bat coronaviruses which replicate in primary human airway epithelial cells. These viruses are poised for emergence because they both efficiently use human ACE2 entry receptors and resist existing vaccines and immunotherapeutics (Menachery et al., Nat Med. 2015 December; 21(12):1508-1; PMID: 26552008). New, highly pathogenic coronaviruses from animal reservoirs are likely to emerge in the future. Many new members of SARS-CoV and MERS-CoV continue to cause a range of effects on the lung tissue, ranging from asymptomatic cases to severe acute respiratory distress syndrome (ARDS) and respiratory failure (Hui et al., Curr Opin Pulm Med. 2014 May; 20(3):233-41; PMID: 24626235).

4. Other Viruses

a. Dengue Virus (DENV)

The disclosure further relates to treatment of Dengue viral (DENV) disease in a subject comprising administering an effective amount of a modulator of complement pathway, e.g., at least one modulator of Table 1. Dengue virus infections, estimated at about 50 million and 390 million annually in over 100 countries, is of paramount significance. Disease caused by DENV infection ranges from asymptomatic, undifferentiated fever and classical dengue fever to severe forms of the disease that include dengue hemorrhagic fever (DHF) and dengue shock syndrome (DSS). An example of a life-threatening outcome of DENV infection is increased vascular permeability and plasma leakage, which ultimately can lead to fatal hypovolemic shock. Studies show a link between DENV infection of macrophages and endothelial cells (EC) in the pathophysiology of capillary leakage and loss of barrier integrity. In certain aspects, macrophages are the major target for DENV replication in vivo, and therefore serve as important sources of cytokines, chemokines, and vasoactive factors that converge on the endothelium to contribute to vascular permeability. The endothelium remains a major site for DENV-mediated pathogenesis.

The complement system is suggested to be involved in DENV disease and particularly in initiation of vascular leakage. In particular, overactivity of the AP due to the low activity of factor H (FH) is thought to be involved in DENV pathogenesis. Several reports have supported an association of overactivity of the complement AP with DENV disease severity, with complement protein consumption, low serum levels of FH, and high levels of factor D (FD) being reported in the circulation of severe DENV patients (Cabeza et al., J Virol. 2018 Jul. 15; 92(14): e00633-18). Particularly, dysregulation of FH production locally within macrophages and EC, the major in vivo sites for DENV replication and pathogenesis, respectively, in combination with elevation of other complement components, such as FB and C3b deposition, are postulated to be associated with increased complement AP activity in Dengue patients. Moreover, clinical and in vivo studies showed that excessive consumption of C3, C4, factor B and C5 contributed to DHF/DSS and increased levels of the products of complement activation (C3a, C5a) that contribute to histamine release, enhanced vascular permeability and vasodilatation in DENV infections. Indeed, the anaphylatoxins concentration in the blood of severe patients correlated with symptoms of vascular leakage.

b. Ross River Virus (RRV)

The disclosure further relates to treatment of Ross River virus (RRV) fever in a subject comprising administering an effective amount of a modulator of complement pathway, e.g., at least one modulator of Table 1. RRV disease symptoms are characterized by debilitating polyarthritis and myositis that frequently results in myalgia and arthralgia. Studies in both humans and mice have identified a critical role for the host inflammatory response in the development of disease and immunopathology following infection, with macrophages playing an essential role in damage to the musculoskeletal system. In particular, Gunn et al. (Virology, 2018 February; 515: 250-260) show that the host complement system through MBL complement pathway plays a critical role in mediating development of disease and tissue damage through activation of inflammatory cells that infiltrate into the muscle and joints following infection. Particularly, the RRV envelope N-linked glycans contribute to MBL deposition and complement activation leading to the development of severe virus-induced damage to the musculoskeletal system. patients. In RRV-induced complement-deficient mouse model, less severe tissue damage and disease symptoms following RRV infection also indicated an important role of complement in the RRV-induced pathogenesis.

c. Influenza Virus (Flu)

The disclosure further relates to treatment of influenza virus-mediated disease in a subject comprising administering an effective amount of a modulator of complement pathway, e.g., at least one modulator of Table 1. Studies with highly pathogenic H5N1 influenza virus infection point to involvement of dysregulated complement activation in the pathogenesis of severe lung damage (Schindler et al., Blood, 76, 1631-1638 (1990); Sun et al., Am. J. Respir. Cell. Mol. Biol. 49, 221-230 (2013)).

5. Methods of Treatment

Inappropriate regulation or activation of the complement pathways may also be implicated in the pathogenesis of viral diseases in subjects.

In some embodiments, the disclosure provides for a method of treating a complement mediated disorder caused by a virus, e.g., coronaviruses such as SARS, MERS, SARS-nCoV-2; DENV, RRV, or flu virus, in a subject (such as a human patient) comprising administering an effective amount of a modulator of the complement system, preferably an inhibitor of a complement pathway target, as provided in Table 1. See also, Thurman et al. (Arthritis Rheumatol. 2017 November; 69(11): 2102-2113); Zelek et al. (Mol Immunol. 2019 October; 114:341-352), the disclosures in which are incorporated by reference herein in their entirety.

TABLE 1
Diagnostic Approaches to the Complement System: Included below are
exemplary approaches to working up patients with suspected viral
diseases, or to provide coverage of the pathways by therapeutics.

| Target | Therapeutic | Developer/Distributor |
|---|---|---|
| C1q | Anti-C1q mAb | Annexon |
| C1 | C1-INH (BERINERT, | CSL Behring, Salix Pharma, Shire, |
| | RUCONEST, CYNRIZE) | respectively |
| C1s | Anti-C1s mAb | True North Therapeutics |
| | BIVV020 activated anti-C1s Ab | Sanofi |
| | C1s peptide | Ra Pharma |
| C2 | PRO-02 mAb | Prothix BV |
| MASP-2 | Anti-MASP-2 mAb | Omeros |
| MASP-3 | Anti-MASP-3 mAb | Omeros |
| Factor D | Anti-FD mAb (Lampalizumab) | Genentech |
| | ACH-4471 or ACH-5228 | Achillion |
| | BCX9930 | Biocryst |
| | Various (see, Maibaum et al. | Novartis |
| | (Nat Chem Biol. 2016 | |
| | 12(12): 1105-10)) | |
| Factor B | Factor B siRNA IONIS-FB-L_{RX} | Ionis |
| | Anti-Factor B mAb | Novelmed/Alexion Pharmaceuticals |
| | LNP023 | Novartis |
| Properdin | Anti-Properdin mAb | Novelmed |
| | CLG561 MAb | Novartis |
| Factor H | AMY-201, ‘mini-factor H’ | Amyndas |
| C3/C5 | Compstatin/Derivative - APL2, 9 | Apellis, |
| Convertases | Compstatin/Derivative - AMY-101 | Amyndas |
| | sCR1/TP10 | Celldex |
| | Mini-FH | Amyndas |
| | Mirococept | AdProTech |
| | CR2-Factor H/TT30 | Alexion Pharmaceuticals |
| C5 | Anti-C5 mAb | Eculizumab |
| C5 - Follow | Eculizumab biosimilar mAbs | Multiple (e.g., ABP 959 (Amgen); Elizaria |
| up | Nomacopan (Coversin; rVA576) | (Generium); SB12 (Samsung)) |
| | RA101495 | Akari |
| | ALN-CC5 | RaPharma |
| | Zimura (ARC1905) | Alnylam |
| | Ravulizumab (ALXN 1210) | IVERIC/Opthotech A |
| | SOBI005 (affibody) | Alexion Pharmaceuticals |
| | Zilucoplan extended release | SOBI |
| | Tesidolumab (LFG316) | RaPharma |
| | Pozelimab | Novartis |
| | Crovalimab (SKY059) | Regeneron |
| | | Roche/Chugai |
| C5 bispecific | ALXN1720 | Alexion Pharmaceuticals |
| C5a/C5aR | Avacopan (CCX-168) | Chemocentryx |
| | Olendalizumab (ALXN1007) | Alexion Pharmaceuticals |
| | BDB-001 | Staidson Biopharm/inflaRx |
| | IFX2 | inflaRx |
| C3a/C3aR | Various | Both experimental and therapeutic |
| C6 | Anti-C6 mAb | Regenesance |
| | C6 Anti-sense RNA | Complement Pharma |
| | CP010 | |
| CD59 | Adeno associated vector (AAV) | Hemera |
| | CAGsCD59 (HMR59) | |

The disclosure particularly relates to use of the following inhibitors of the C5/C5a axis in the therapy of viral diseases or symptoms related thereto.

In certain aspects, a method is provided of treating a complement mediated disorder caused by a virus that can cause lung or pulmonary injury in a subject (i.e., inflammation of cells in the large airway and parenchyma; (2) perivascular cuffing; (3) thickening of the interstitial membrane; and/or (4) intra-alveolar edema), comprising administering an effective amount of an inhibitor of a complement C5 protein (“a C5 inhibitor”) to the subject. In some embodiments, the virus causing lung or pulmonary injury includes coronavirus such as SARS-CoV, MERS-CoV, or SARS-CoV-2 (2019-nCoV) or flu virus.

In some aspects, the human subject is suffering from severe viral disease comprising shortness of breath (e.g., resting rate>30 breaths/minute; oxygen saturation<93% at rest or arterial oxygen partial pressure (PaO2)/fraction of inspired oxygen (FiO2)<300 mmHg (1 mmHg=0.133 kPa)). In some aspects, the human subject is suffering from critical viral disease comprising respiratory failure requiring mechanical ventilation; respiratory shock; severe pneumonia; acute lung injury (ALI); ARDS requiring oxygen supplementation; and/or combined failure of non-respiratory organs (e.g., heart, kidney) that require ICU monitoring. In some aspects, the human subject is suffering from critical viral disease displays at least one symptom selected from (a) progressive reduction of peripheral blood lymphocytes; (b) progressive increase of peripheral inflammatory cytokines such as IL-6 and C-reactive protein; (c) progressive increase of lactate; and (d) rapid progression of lung pathologies in a short period of time.

In certain aspects, a method is provided of treating lung or pulmonary injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the treatment of lung or pulmonary injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating connective or skeletal tissue injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the virus causing lung or pulmonary injury includes Ross River virus (RRV). In some embodiments, the treatment of connective or skeletal tissue injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating endothelial or vascular injury in a subject, comprising determining that the C5a level is elevated in the subject, and administering an effective amount of a C5 inhibitor, such as, for example, eculizumab, an antigen-binding fragment thereof, an antigen-binding variant thereof (also referred to herein as an eculizumab variant or a variant eculizumab), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the subject. In some embodiments, the virus causing endothelial or vascular injury includes Dengue virus (DENV). In some embodiments, the treatment of endothelial or vascular injury in a subject, comprises administering an effective amount of a C5a inhibitor, such as, for example, olendalizumab (ALXN 1007), an antigen-binding fragment thereof, an antigen-binding variant thereof, a polypeptide comprising the antigen-binding fragment of olendalizumab (ALXN 1007) or the antigen-binding fragment of the variant, a fusion protein comprising the antigen binding fragment of olendalizumab (ALXN 1007) or the antigen-binding fragment of the variant, or a single chain antibody version of olendalizumab (ALXN 1007) or of the variant thereof, to the subject.

In certain aspects, a method is provided of treating a subject with coronaviral disease, e.g., 2019-nCoV acute respiratory disease (COVID-19), the method comprising administering to the subject an effective amount of an anti-C5 antibody, or antigen binding fragment thereof, wherein the method comprises an administration cycle comprising an induction phase followed by a maintenance phase, wherein: the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or the anti-C5 antibody, or antigen binding fragment thereof, is administered during the induction phase at a dose of 600 mg weekly for 1 week, starting at day 0, and is administered during the maintenance phase at a dose of 600 mg every week; or the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

In certain aspects, a method for treating a complement mediated disorder caused by a virus (e.g., coronavirus such as SARS-CoV, MERS-CoV, or SARS-CoV-2 (2019-nCoV); Dengue virus (DENV); Ross River virus (RRV) and/or influenza virus (flu)) in a human subject is provided, wherein the method comprises intravenously administering eculizumab at a dose of 900 mg on Days 1, 8, 15, and 22. In one embodiment, the method further comprises administering eculizumab at a dose of 900 mg on Day 4, Day 12, and Day 18.

In some embodiments, the method comprises monitoring complement (e.g., CH50, C3, C4, C4d, sC5b9, C5) and residual eculizumab plasma levels before, during, and after the treatment period. In one embodiment, the method comprises monitoring complement (e.g., CH50, C3, C4, C4d, sC5b9, C5) and residual eculizumab plasma levels before each administration of eculizumab and at Day 1, Day 2, Day 3, and Day 6 to ensure satisfactory drug exposition. In some embodiments, the treatment eliminates the need for intubation (e.g., at day 14). In other embodiments, the treatment results in an improvement on the OMS progression scale compared to baseline. In other embodiments, the treatment results in an improvement on the OMS progression scale at Days 4, 7, and/or 14 compared to baseline. In other embodiments, the treatment results in a decreased time to discharge. In other embodiments, the treatment results in a decreased time to oxygen supply independency. In other embodiments, the treatment results in a decreased time to negative viral excretion. In other embodiments, the treatment results in an improvement in one or more biological parameters (e.g., C5b9, estimated GFR, CRP, myoglobin, CPK, cardiac troponin, ferritin, lactate, cell blood count, liver enzymes, LDH, D-Dimer, albumin, fibrinogen, triglycerides, coagulation tests, urine electrolyte, creatinuria, proteinuria, uricemia, IL6, procalcitonin, immunophenotype and/or exploratory tests).

In other embodiments, the patient requires hospitalization and/or treatment in an intensive care unit (ICU). In some embodiments, the treatment results in a decrease in organ failure at Day 3 (e.g., as defined by the relative variation in Sequential Organ Failure Assessment (SOFA) score at Day 3) in the ICU patient. In other embodiments, the treatment results in a decrease or elimination of secondary infections (e.g., pneumonia acquired) in the ICU patient. In other embodiments, the treatment results in vasopressor free survival (e.g., pneumonia acquired) in the ICU patient. In other embodiments, the treatment results in ventilator free survival in the ICU patient. In other embodiments, the treatment results in a decreased incidence of dialysis in the ICU patient. In other embodiments, the treatment results in an improvement on the OMS progression scale for the ICU patient compared to baseline. In other embodiments, the treatment results in an improvement on the OMS progression scale for the ICU patient compared to baseline at Days 4, 7 and 14 days, overall survival at 14, 28 and 90 days, 28-day ventilator free-days, improved evolution of PaO2/FiO2 ratio, decreased respiratory acidosis at day 4 (arterial blood pH of <7.25 with a partial pressure of arterial carbon dioxide [Paco2] of ≥60 mm Hg for >6 hours), decreased time to oxygen supply independency, decreased duration of hospitalization, decreased time to negative viral excretion, and/or decreased time to ICU and hospital discharge. In other embodiments, the treatment results in an improvement in one or more of the following biological parameters for the ICU patient: sC5b9, estimated GFR, CRP, cardiac troponin, urine electrolyte and creatinine, proteinuria, uricemia, IL6, myoglobin, KIM-1, NGAL, CPK, ferritin, lactate, cell blood count, liver enzymes, LDH, D-Dimer, albumin, fibrinogen, triglycerides, coagulation tests (including activated partial thromboplastin time), procalcitonin, immunophenotype, exploratory tests, rate of renal replacement therapy, and/or ventilation parameters.

In certain aspects, a method for treating a subject with coronaviral disease (e.g., COVID-19) is provided, wherein the method comprises intravenously administering eculizumab at a dose of 1200 mg on Days 1, 4, and 8. In some embodiments, the method comprises intravenously administering eculizumab at a dose of 1200 mg on Days 1, 4, and 8 and 900 mg on Days 15 and 22. In other embodiments, the method comprises intravenously administering eculizumab (a) at a dose of 1200 mg on Days 1, 4, and 8, (b) at a dose of 900 mg on Days 15 and 22, and (c) at a dose of 900 mg or 1200 mg on Days 12 and 18. In some embodiments, eculizumab is administered based on the therapeutic dose monitoring (TDM). In some embodiments, TDM comprises monitoring of a parameter selected from eculizumab plasma level and free C5 free C-5, and/or CH50 suppression, wherein, the optional dose is administered if the parameter is modulated (e.g., attenuated) compared to a reference standard. In some embodiments, the treatment results in improved mechanical ventilation status, improved oxygen saturation levels (SpO2 and/or PaO2), improved supplemental oxygen status, decreased time in the intensive care unit, and/or decreased duration of hospitalization.

In certain aspects, a method is provided for treating a subject with coronaviral disease, e.g., COVID-19, comprising intravenously administering ravulizumab on Day 1 based on weight-based loading dose per label (e.g., United States Product Insert (USPI) label for ULTOMIRIS® (ravulizumab-cwvz) injection, for intravenous use; Initial U.S. Approval: 2018; Revised: 10/2019); intravenously administering 900 mg (or 600 mg for patients<60 kg) on day 5 (D5); intravenously administering 900 mg (or 600 mg for patients<60 kg) of ravulizumab on Day 10 (D10) and intravenously administering 900 mg of ravulizumab for all patients on Day 15 (D15). For example, in one embodiment, ravulizumab is administered to the patient on Day 5 and Day 10 at a dose of 600 mg or 900 mg (based on weight category) and then on Day 15 at a dose of 900 mg. Specifically, a weight-based dose is administered on Day 1 as follows: Patients weighing≥40 to <60 kg: 2400 mg/kg; ≥60 to <100 kg: 2700 mg/kg; or ≥100 kg: 3000 mg/kg on Day 1. On Day 5 and Day 10, doses of 600 mg or 900 mg ravulizumab are administered (according to weight category) and on Day 15 patients receive 900 mg ravulizumab. Final assessment is performed at Day 29 or on day of discharge, whichever occurs first. Screening and the Day 1 visits can occur on the same day if the patient has met all inclusion and no exclusion criteria. In some embodiments, the treatment improves the survival rate of patients with SARS CoV 2 infection who are receiving ravulizumab plus best supportive care (BSC) compared with BSC alone. In other embodiments, the treatment decreases lung injury in patients with SARS CoV 2 infection while on supportive medical care. In other embodiments, the treatment improves clinical outcomes in patients with SARS CoV 2 infection while on supportive medical care. In some embodiments, the treatment results in one ore more of the following: (1) a decrease number of days free of mechanical ventilation at Day 29, (2) decreased duration of intensive care unit stay at Day 29, (3) improved change from baseline in sequential organ failure assessment at Day 29, (4) improved change from baseline in SpO2/FiO2 at Day 29, (5) decreased duration of hospitalization at Day 29, and/or (5) survival (based on all-cause mortality) at Day 60 and Day 90.

In certain aspects, a method of treating a subject with coronaviral disease (e.g., COVID-19) is provided, wherein the method comprises administering eculizumab to the patient according to a uniform schedule of eculizumab (e.g., 4 doses of 1200 mg every 3 days, followed by 3 doses of 900 mg every 3 days) until oxygen support independence. In some embodiments, the patient is an intubated patient (e.g., severe, non-ICU).

In certain aspects, a method of treating severe coronavirus disease-2019 (COVID-19) in a human patient infected with SARS-CoV-2 (2019-nCoV) is provided, wherein the method comprises administering an effective amount a pharmaceutical composition comprising eculizumab (SOLIRIS®). In one embodiment, the severe COVID-19 comprises a need for hospitalization and/or treatment in an intensive care unit (ICU).

In certain aspects, a method of effectively treating severe coronavirus disease-2019 (severe COVID-19) in a human patient with eculizumab is provided, wherein the method comprises: (a) measuring a level of a marker which is C5b-9 (membrane attack complex; MAC) in the patient's blood sample, prior to and after treatment with eculizumab; (b) comparing the marker level to a reference standard; (c) titrating the treatment dose of eculizumab until the marker level in the human patient converges towards the reference standard; and (d) administering the titrated dose of eculizumab to the human patient. In one embodiment, the marker is circulating sC5b9 level and the reference standard comprises a level of about 340 ng/ml, wherein the effective treatment comprises reduction in duration of hospitalization and/or length of intensive care unit (ICU) stay. In another embodiment, the marker comprises circulating sC5b9 level and the reference standard comprises a level of about 340 ng/ml, wherein a positive differential (e.g., sC5b9 levels in the patient's sample>about 340 ng/ml) indicates longer hospitalization and/or ICU stays.

In certain aspects, a method of prognosticating an outcome which is duration of hospitalization and/or treatment in an intensive care unit (ICU) in a human patient inflicted with severe coronavirus disease-2019 (severe COVID-19) is provided, wherein the method comprises measuring a level of a marker which is C5b-9 (membrane attack complex; MAC) in the patient's blood sample, wherein an increase in marker level compared to a reference standard is prognostic of the outcome.

6. C5 Inhibitors

A C5 inhibitor (an inhibitor of complement C5 protein) for use in a method or a kit disclosed herein can be any C5 inhibitor. In certain embodiments, the C5 inhibitor for use in methods and kits disclosed herein is a polypeptide inhibitor. In certain embodiments, the C5 inhibitor is eculizumab, an antigen-binding fragment thereof, a polypeptide comprising the antigen-binding fragment of eculizumab, a fusion protein comprising the antigen binding fragment of eculizumab, or a single chain antibody version of eculizumab, or a small-molecule C5 inhibitor. In certain embodiments, the C5 inhibitor is ravulizumab, an antigen-binding fragment thereof, a polypeptide comprising the antigen-binding fragment of ravulizumab, a fusion protein comprising the antigen binding fragment of ravulizumab, or a single chain antibody version of ravulizumab, or a small-molecule C5 inhibitor.

In some embodiments, the C5 inhibitor is a molecule that binds to a complement C5 protein and is also capable of inhibiting the generation of C5a. A C5-binding inhibitor can also be capable of inhibiting, e.g., the cleavage of C5 to fragments C5a and C5b, and thus preventing the formation of terminal complement complex. For example, an anti-C5 antibody blocks the generation or activity of the C5a active fragment of a C5 protein (e.g., a human C5 protein). Through this blocking effect, the antibody inhibits, e.g., the proinflammatory effects of C5a. An anti-C5 antibody can further have activity in blocking the generation or activity of C5b. Through this blocking effect, the antibody can further inhibit, e.g., the generation of the C5b-9 membrane attack complex at the surface of a cell.

In some embodiments, the C5 inhibitor is a polypeptide inhibitor which is eculizumab or a variant thereof. Eculizumab is a humanized anti-human C5 monoclonal antibody (Alexion Pharmaceuticals, Inc.), with a human IgG2/IgG4 hybrid constant region, to reduce the potential to elicit proinflammatory responses. Eculizumab has the trade name SOLIRIS®. Eculizumab further blocks the formation of the terminal complement complex. See, e.g., Hillmen et al., N Engl J Med 2004; 350:552-9; Rother et al., Nature Biotechnology 2007; 25(11): 1256-1264; Hillmen et al., N Engl J Med 2006, 355; 12, 1233-1243; Zuber et al., Nature Reviews Nephrology 8, 643-657 (2012); U.S. Pat. Nos. 6,355,245; 9,718,880; 9,725,504.

In yet further other embodiments, the C5 inhibitor is a single chain version of eculizumab. See, e.g., Whiss (2002) Curr Opin Investig Drugs 3(6):870-7; Patel et al. (2005) Drugs Today (Barc) 41(3):165-70; Thomas et al. (1996) Mol Immunol 33(17-18):1389-401; and U.S. Pat. No. 6,355,245.

In certain embodiments, the anti-C5 antibody is a variant derived from eculizumab, having one or more improved properties (e.g., improved pharmacokinetic properties) relative to eculizumab. The variant eculizumab antibody (also referred to herein as an eculizumab variant, a variant eculizumab, or the like) or C5-binding fragment thereof is one that: (a) binds to complement component C5; (b) inhibits the generation of C5a; and can further inhibit the cleavage of C5 into fragments C5a and C5b. See, e.g., U.S. Pat. No. 9,079,949 and WO2015134894.

In some embodiments, a C5-binding polypeptide for use in methods of this disclosure is not a whole antibody. In some embodiments, a C5-binding polypeptide is a single chain antibody. In some embodiments, a C5-binding polypeptide for use in methods of this disclosure is a bispecific antibody. In some embodiments, a C5-binding polypeptide for use in methods of this disclosure is a humanized monoclonal antibody, a chimeric monoclonal antibody, or a human monoclonal antibody, or an antigen binding fragment of any of them.

In yet other embodiments, the C5 inhibitor is LFG316 (Novartis, Basel, Switzerland, and MorphoSys, Planegg, Germany) or another antibody defined by the sequences of Table 1 in U.S. Pat. Nos. 8,241,628 and 8,883,158, ARC1905 (Ophthotech, Princeton, NJ and New York, NY), which is an anti-C5 pegylated RNA aptamer (see, e.g., Keefe et al., Nature Reviews Drug Discovery 9, 537-550 (July 2010) doi:10.1038/nrd3141), Mubodina® (Adienne Pharma & Biotech, Bergamo, Italy) (see, e.g., U.S. Pat. No. 7,999,081), rEV576 (coversin) (Volution Immuno-pharmaceuticals, Geneva, Switzerland) (see, e.g., Penabad et al., Lupus, 2014 October; 23(12):1324-6), ARC1005 (Novo Nordisk, Bagsvaerd, Denmark), SOMAmers (SomaLogic, Boulder, CO), SOB1002 (Swedish Orphan Biovitrum, Stockholm, Sweden), RA101348 (Ra Pharmaceuticals, Cambridge, MA), Aurin Tricarboxylic Acid (“ATA”), and anti-C5-siRNA (Alnylam Pharmaceuticals, Cambridge, MA), and Ornithodoros moubata C inhibitor (‘OmCI”).

In some embodiments, the polypeptide C5 inhibitor is an antibody (referred to herein as an “anti-C5 antibody,” C-5 binding antibody, or the like), or an antigen binding fragment thereof. The antibody can be a monoclonal antibody. In other embodiments, the polypeptide C5 inhibitor comprises the variable region, or a fragment thereof, of an antibody, such as a monoclonal antibody. In other embodiments, the polypeptide C5 inhibitor is an immunoglobulin that binds specifically to a C5 complement protein. In other embodiments, the polypeptide inhibitor is an engineered protein or a recombinant protein, as defined hereinabove. In some embodiments, a C5-binding polypeptide is not a whole antibody but comprises parts of an antibody. In some embodiments, a C5-binding polypeptide is a single chain antibody. In some embodiments, a C5-binding polypeptide is a bispecific antibody. In some embodiments, the C5-binding polypeptide is a humanized monoclonal antibody, a chimeric monoclonal antibody, or a human monoclonal antibody, or an antigen binding fragment of any of them. Methods of making a polypeptide C5 inhibitor, including antibodies, are known in the art.

As stated above, the C5 inhibitor, including a C5-binding polypeptide, can inhibit complement component C5. In particular, the inhibitors, including polypeptides, inhibit the generation of the C5a anaphylatoxin, or the generation of c5a and the C5b active fragments of a complement component C5 protein (e.g., a human C5 protein). Accordingly, the C5 inhibitors inhibit, e.g., the pro-inflammatory effects of C5a; and can inhibit the generation of the C5b-9 membrane attack complex (“MAC”) at the surface of a cell and subsequent cell lysis. See, e.g., Moongkarndi et al. (1982) Immunobiol 162:397 and Moongkarndi et al. (1983) Immunobiol 165:323.

Suitable methods for measuring inhibition of C5 cleavage are known in the art. For example, the concentration and/or physiologic activity of C5a and/or C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 known in the art can be used. Other assays known in the art can also be used.

The anti-C5 antibodies described herein and used for the methods and kits disclosed herein bind to complement component C5 (e.g., human C5) and inhibit the cleavage of C5 into fragments C5a and C5b.

In certain aspects, the anti-C5 antibody or a variant thereof or the antigen-binding fragment thereof is administered to the subject in an administration cycle comprising an induction phase followed by a maintenance phase, wherein: the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or the anti-C5 antibody, or antigen binding fragment thereof, is administered during the induction phase at a dose of 600 mg weekly for 1 week, starting at day 0, and is administered during the maintenance phase at a dose of 600 mg every week; or the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

In certain aspects, a method is provided for treating a subject with coronaviral disease, e.g., COVID-19, comprising intravenously administering eculizumab at a dose of 1200 mg on Days 1, 4, and 8; optionally administering 900 mg or 1200 mg of eculizumab at day 12 (D12) based on the therapeutic dose monitoring (TDM); administering 900 mg dose intravenously on day 15 (D15); optionally administering 900 mg or 1200 mg of intravenous eculizumab at day 18 (D18) based on TDM; and administering 900 mg dose intravenously on day 22 (D22). Preferably, TDM comprises monitoring of a parameter selected from eculizumab plasma level and free C5 free C-5, and/or CH50 suppression, wherein, the optional dose is administered if the parameter is modulated (e.g., attenuated) compared to a reference standard.

In certain aspects, a method is provided for treating a subject with coronaviral disease, e.g., COVID-19, comprising intravenously administering ravulizumab on Day 1 based on weight-based loading dose per label (e.g., United States Product Insert (USPI) label for ULTOMIRIS® (ravulizumab-cwvz) injection, for intravenous use; Initial U.S. Approval: 2018; Revised: 10/2019); intravenously administering 900 mg (or 600 mg for patients<60 kg) on day 5 (D5); intravenously administering 900 mg (or 600 mg for patients<60 kg) of ravulizumab on Day 10 (D10) and intravenously administering 900 mg of ravulizumab for all patients on Day 15 (D15).

a. Anti-C5 Antibodies

Anti-C5 antibodies (or VH/VL domains derived therefrom or CDRs comprising antigen-binding domains thereof) suitable for use in the invention can be generated using methods well known in the art. Alternatively, art recognized anti-C5 antibodies can be used. Antibodies that compete with any of these art-recognized antibodies for binding to C5 also can be used, including biosimilars of art-known antibodies.

The present disclosure relates to, inter alia, antibodies, or antigen-binding fragments thereof, that bind to C5 and use of such antibodies or antigen-binding fragments in methods for treating or preventing complement-associated viral disorders such as, but not limited to, COVID-19, SARS, MERS, Dengue fever, Ross River fever, and influenza. Preferably, the anti-C5 antibodies and antigen-binding fragments thereof used in the treatment of the above viral disorders are those disclosed in WO1995029697 and corresponding U.S. Pat. Nos. 6,074,642; 6,355,245; and corresponding EP Pat. No. 0758904B1, the disclosures in the documents, including the antibody sequences (e.g., VHCDR₁₋₃ and VLCDR₁₋₃ of the antibodies and also the complete VH/VL chains thereof), are incorporated herein by reference.

In some embodiments, compositions containing the anti-C5 antibodies and antigen-binding fragments thereof used in the treatment of the above viral disorders are those disclosed in WO2007106585 and corresponding U.S. Pat. No. 9,732,149; and corresponding EP Pat. No. 2359834B1 and EP Pub. No. EP3124029A1, the disclosures in the documents, including the antibody sequences (e.g., VHCDR₁₋₃ and VLCDR₁₋₃ of the antibodies and also the complete VH/VL chains thereof), are incorporated herein by reference. In some embodiments, compositions containing the anti-C5 antibodies and antigen-binding fragments thereof used in the treatment of the above viral disorders are those disclosed in WO2008069889 and corresponding US Pub. No. 2007/0116710A1 and corresponding EP Pub. No. 2089058A2, the disclosures in the documents, including the antibody sequences (e.g., VHCDR₁₋₃ and VLCDR₁₋₃ of the antibodies and also the complete VH/VL chains thereof), are incorporated herein by reference.

In some embodiments, the disclosure relates to eculizumab, variable heavy (VH) and/or variable light (VL) chains of eculizumab, or antigen-binding fragments thereof comprising complementarity determining regions (CDRs) of the heavy (VH) and light (VL) chains (e.g., VHCDR₁₋₃ and VLCDR₁₋₃ of eculizumab). Eculizumab (also known as SOLIRIS®) is an anti-C5 antibody comprising heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. Eculizumab comprises a heavy chain variable region having the amino acid sequence set forth in SEQ ID NO: 7 and a light chain variable region having the amino acid sequence set forth in SEQ ID NO: 8. Eculizumab comprises a heavy chain comprising the amino acid sequence set forth in SEQ ID NO:10 and a light chain having the amino acid sequence set forth in SEQ ID NO:11.

Another exemplary anti-C5 antibody is ULTOMIRIS® (ravulizumab) comprising heavy and light chains having the sequences shown in SEQ ID NOs:14 and 11, respectively, or antigen binding fragments and variants thereof. Ravulizumab (also known as BNJ441 and ALXN1210) is described in PCT/US2015/019225 and U.S. Pat. No. 9,079,949, the teachings or which are hereby incorporated by reference. The terms ULTOMIRIS®, ravulizumab, BNJ441, and ALXN1210 may be used interchangeably throughout this document. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. Accordingly, in one embodiment, the antibody comprises the CDR1, CDR2, and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab (BNJ441) and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, IL, CC Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

b. Anti-C5 Bispecific Minibodies

The present disclosure relates to, inter alia, bispecific antibodies or minibodies thereof, that bind to C5 and use of such bispecific antibodies or minibodies in methods for treating or preventing complement-associated viral disorders such as, but not limited to, COVID-19, SARS, MERS, Dengue fever, Ross River fever, and influenza. Preferably, the anti-C5 bispecific antibodies or minibodies used in the treatment of the above viral disorders comprise engineered polypeptides that specifically bind to human complement component C5 and/or serum albumin. Representative examples include those disclosed in Int. App. No. PCT/US2018/041661 (published as WO2019014360) and corresponding U.S. Ser. No. 16/629,687; and corresponding EP Ser. No. 18746529.9, the disclosures in the documents, including the sequences of the bispecific minibodies, are incorporated herein by reference. Preferably, the disclosure relates to anti-C5 bispecific ALXN 1720, including variants thereof.

c. Anti-C5a Antibodies

The present disclosure relates to, inter alia, antibodies, or antigen-binding fragments thereof, that bind to C5a and use of such antibodies or antigen-binding fragments in methods for treating or preventing complement-associated viral disorders such as, but not limited to, COVID-19, SARS, MERS, Dengue fever, Ross River fever, and influenza. Preferably, the anti-C5a antibodies and antigen-binding fragments thereof used in the treatment of the above viral disorders are those disclosed in WO2011137395 and corresponding U.S. Pat. Nos. 9,011,852; 9,371,378; 10,450,370; and corresponding EP Pat. No. 2563813B1 and EP Pat. No. 2824111B1, the disclosures in the documents, including the antibody sequences (e.g., VHCDR₁₋₃ and VLCDR₁₋₃ of the antibodies and also the complete VH/VL chains thereof), are incorporated herein by reference. Preferably, the disclosure relates to olendalizumab (ALXN 1007), variable heavy (VH) and/or variable light (VL) chains of olendalizumab or antigen-binding fragments thereof comprising complementarity determining regions (CDRs) of the heavy (VH) and light (VL) chains (e.g., VHCDR₁₋₃ and VLCDR₁₋₃ of olendalizumab).

In certain aspects, a method is provided of treating a complement mediated disorder caused by a coronavirus in a subject (such as a human patient) comprising administering an effective amount of a polypeptide inhibitor of complement C5 protein (such as human complement C5 protein) to the subject.

In certain embodiments, the coronaviral disorder is caused by a coronavirus that can cause lung injury in a subject. In certain embodiments, the coronaviral disorder causes respiratory illness that ranges from mild to severe or even deadly. In certain embodiments, the coronaviral disorder produces at least one symptom selected from fever, cough or shortness of breath.

In certain embodiments, a therapeutically effective amount of a C5 inhibitor (such as eculizumab) can include an amount (or various amounts in the case of multiple administration) that improves the subject's chance of survival. In certain embodiments, a disclosed method improves the life expectancy of a subject by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

In certain embodiments, a therapeutically effective amount of a C5 inhibitor (such as eculizumab or ravulizumab) can include an amount (or various amounts in the case of multiple administration) that decreases hemolysis, decreases disseminated intravascular coagulation, increases platelet levels, reduces complement levels, decreases levels of the cytokines that are over-produced, inhibits thrombolytic microangiopathy, maintains or improves renal functions, or reduces other symptoms of the disease (such as fever), or any combination thereof. These parameters can be ascertained or measured by any methods known in the art.

For example, methods for determining whether a particular C5 inhibitor, such as an anti-C5 antibody, inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2nd Edition,” 135-240, Springfield, IL, CC Thomas (1961), pages 135-139, or a conventional variation of that assay such as the chicken erythrocyte hemolysis method as described in, e.g., Hillmen et al. (2004) N Engl J Med 350(6):552. Methods for determining whether a compound inhibits the cleavage of human C5 into forms C5a and C5b are known in the art and described in, e.g., Moongkarndi et al. (1982) Immunobiol 162:397; Moongkamdi et al. (1983) Immunobiol 165:323; Isenman et al. (1980) J Immunol 124(1):326-31; Thomas et al. (1996) Mol Immunol 33(17-18):1389-401; and Evans et al. (1995) Mol Immunol 32(16):1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 known in the art can be used. Other assays known in the art can also be used.

Immunological techniques such as, but not limited to, ELISA can be used to measure the protein concentration of C5 and/or its split products to determine the ability of a C5 inhibitor, such as an anti-C5 antibody, to inhibit conversion of C5 into biologically active products. For example, C5a generation can be measured. Also, as another example, C5b-9 neoepitope-specific antibodies can be used to detect the formation of terminal complement.

Hemolytic assays can be used to determine the inhibitory activity of a C5 inhibitor, such as an anti-C5 antibody, on complement activation. In order to determine the effect of a C5 inhibitor, such as an anti-C5 antibody, on classical complement pathway-mediated hemolysis in a serum test solution in vitro, for example, sheep erythrocytes coated with hemolysin or chicken erythrocytes sensitized with anti-chicken erythrocyte antibody can be used as target cells. The percentage of lysis is normalized by considering 100% lysis equal to the lysis occurring in the absence of the inhibitor. Also, the classical complement pathway can be activated by a human IgM antibody, for example, as utilized in the Wieslab® Classical Pathway Complement Kit (Wieslab® COMPL CP310, Euro-Diagnostica, Sweden). Briefly, the test serum is incubated with, for example, a C5 inhibitor such as an anti-C5 antibody in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the C5 inhibitor, such as an anti-C5 antibody. In some embodiments, the test serum is a C5-deficient serum reconstituted with a C5 polypeptide.

To determine the effect of a C5 inhibitor, such as an anti-C5 antibody, on alternative pathway-mediated hemolysis, unsensitized rabbit or guinea pig erythrocytes can be used as the target cells. The serum test solution is a C5-deficient serum reconstituted with a C5 inhibitor, such as an anti-C5 polypeptide. The percentage of lysis is normalized by considering 100% lysis equal to the lysis occurring in the absence of the inhibitor. The alternative complement pathway can be activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with a C5 inhibitor, such as an anti-C5 antibody, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the C5 inhibitor, such as an anti-C5 antibody.

C5 activity, or inhibition thereof, can be quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (“TCC”) formation, since the TCC themselves are directly responsible for the hemolysis that is measured. The assay is well known and commonly practiced by those skilled in the art.

Briefly and for example, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL). Inhibition, e.g., as it pertains to terminal complement activity, includes at least an about 5 (e.g., at least an about 6, about 7, about 8, about 9, about 10, about 15, about 20, about 25, about 30, about 35, about 40, about 45, about 50, about 55, about or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least about 40% (e.g., at least about 45, about 50, about 55, about 60, about 65, about 70, about 75, about 80, about 85, about 90, about 95, or up to about 100%).

In certain embodiments, the anti-C5 antibody, or antigen binding fragment thereof, is administered during the induction phase at a dose of 900 mg weekly for 4 weeks, starting at day 0, and is administered during the maintenance phase at a dose of 1200 mg in week 5 (day 28) and then 1200 mg every two weeks, wherein the human subject is greater than or equal to 40 kg. In certain embodiments, the anti-C5 antibody, or antigen binding fragment thereof, is administered during the induction phase at a dose of 600 mg weekly for 2 weeks, starting at day 0, and is administered during the maintenance phase at a dose of 900 mg in week 3 (day 14), and then 900 mg every two weeks, wherein the human subject is between 30 kg and 40 kg. In certain embodiments, the anti-C5 antibody, or antigen binding fragment thereof, is administered during the induction phase at a dose of 600 mg weekly for 2 weeks, starting at day 0, and is administered during the maintenance phase at a dose of 600 mg in week 3 (day 14), and then 600 mg every two weeks, wherein the human subject is between 20 kg and 30 kg. In certain embodiments, the anti-C5 antibody, or antigen binding fragment thereof, is administered during the induction phase at a dose of 600 mg weekly for 1 week, starting at day 0, and is administered during the maintenance phase at a dose of 600 mg every week (starting from day 7), wherein the human subject is between 10 kg and 20 kg.

In certain embodiments, the anti-C5 antibody, or antigen binding fragment thereof, is administered during the induction phase at a dose of 300 mg weekly for 1 week, starting at day 0, and is administered during the maintenance phase at a dose of 300 mg at week 2 (day 7) and then every 3 weeks, wherein the human subject is between 5 kg and 10 kg.

In some embodiments, the treatment method maintains a serum trough concentration of the anti-C5 antibody, or antigen binding fragment thereof, of about 35 μg/mL to about 700 μg/mL during the induction phase and/or the maintenance phase.

The anti-C5 antibody, or antigen binding fragment thereof, may be formulated for intravenous administration, including administration as an IV infusion. In some embodiments, the subject has not previously been treated with a complement inhibitor. The administration cycle can be 8 weeks; or it can be 16 weeks.

7. Pharmaceutical Compositions and Formulations

The disclosure also relates to use of pharmaceutical compositions comprising modulators of the complement system (e.g., one or more compounds of Table 1) and a pharmaceutically acceptable carrier. For instance, compositions containing a C5 inhibitor, such as a C5-binding polypeptide, can be formulated as a pharmaceutical composition for administering to a subject. Any suitable pharmaceutical compositions and formulations, as well as suitable methods for formulating and suitable routes and suitable sites of administration, are within the scope of this invention, and are known in the art. Also, unless otherwise stated, any suitable dosage(s) and frequency of administration are contemplated.

The pharmaceutical compositions can include a pharmaceutically acceptable carrier (i.e., an excipient). A “pharmaceutically acceptable carrier” refers to, and includes, any and all solvents, dispersion media, coatings, antibacterial and antifungal agents, isotonic and absorption delaying agents, diluent, glidant, etc. The compositions can include a pharmaceutically acceptable salt, e.g., an acid addition salt or a base addition salt (see e.g., Berge et al. (1977) J Pharm Sci 66:1-19). The composition can be coated when appropriate.

In certain embodiments, the protein compositions can be stabilized and formulated as a solution, microemulsion, dispersion, liposome, lyophilized (freeze-dried) powder, or other ordered structure suitable for stable storage at high concentration. Sterile injectable solutions can be prepared by incorporating a C5-binding polypeptide, for use in the methods of this invention, in the required amount in an appropriate solvent with one or a combination of ingredients enumerated above, as required, followed by filtered sterilization. Generally, dispersions are prepared by incorporating a C5-binding polypeptide into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a C5 inhibitor polypeptide plus any additional desired ingredient from a previously sterile-filtered solution thereof. The proper fluidity of a solution can be maintained, for example, using a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin. Non-protein C5 inhibitors can be formulated in the same, or similar, way.

The C5 inhibitor, including a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated at any desired concentration, including relatively high concentrations in aqueous pharmaceutical solutions. For example, a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in solution at a concentration of between about 10 mg/mL to about 100 mg/mL (e.g., between about 9 mg/mL and about 90 mg/mL; between about 9 mg/mL and about 50 mg/mL; between about 10 mg/mL and about 50 mg/mL; between about 15 mg/mL and about 50 mg/mL; between about 15 mg/mL and about 110 mg/mL; between about 15 mg/mL and about 100 mg/mL; between about 20 mg/mL and about 100 mg/mL; between about 20 mg/mL and about 80 mg/mL; between about 25 mg/mL and about 100 mg/mL; between about 25 mg/mL and about 85 mg/mL; between about 20 mg/mL and about 50 mg/mL; between about 25 mg/mL and about 50 mg/mL; between about 30 mg/mL and about 100 mg/mL; between about 30 mg/mL and about 50 mg/mL; between about 40 mg/mL and about 100 mg/mL; between about 50 mg/mL and about 100 mg/mL; or between about 20 mg/mL and about 50 mg/mL); or at any suitable concentration. A C5-binding polypeptide used in the methods of this invention can be present in the solution at greater than (or at least equal to) about 5 (e.g., greater than, or at least equal to, about any of the following: 5, 6, 7, 8, 9, 10, 11, 12, 13, 14, 15, 16, 17, 18, 19, 20, 21, 22, 23, 24, 25, about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, or even 150) mg/mL. A C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated at a concentration of greater than about 2 (e.g., greater than about any of the following: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or more) mg/mL, but less than about 101 (e.g., less than about any of the following: 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or less than about 5) mg/mL. Thus, in some embodiments, a C5-binding polypeptide used in the methods of this invention, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in an aqueous solution at a concentration of greater than about 5 mg/mL and less than about 100 mg/mL. A C5-binding polypeptide used in the methods of this invention, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in an aqueous solution at a concentration of about 10 mg/mL or 50 mg/mL or 100 mg/mL. Any suitable concentration is contemplated. Methods for formulating a protein in an aqueous solution are known in the art and are described in, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (1995), “Therapeutic peptides and proteins: formulation, processing, and delivery systems,” CRC Press.

Unless otherwise noted, the dosage level for a C5 inhibitor can be any suitable level. In certain embodiments, the dosage levels of an C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, for human subjects can generally be between about 1 mg per kg and about 100 mg per kg per subject per treatment, and can be between about 5 mg per kg and about 50 mg per kg per subject per treatment.

The plasma concentration in a subject, whether the highest level achieved or a level that is maintained, of a C5 inhibitor can be any desirable or suitable concentration. Such plasma concentration can be measured by methods known in the art. Such a plasma concentration of an anti-C5 antibody, in a subject can be the highest attained after administering the anti-C5 antibody or can be a concentration of an anti-C5 antibody in a subject that is maintained throughout the therapy. However, greater amounts (concentrations) may be required for extreme cases and smaller amounts may be sufficient for milder cases; and the amount can vary at different times during therapy. In some embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM, or at or above between about 280 nM to 285 nM, during treatment.

In certain embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM to about 430 nM, or at or above about 570 nM to about 580 nM, during treatment.

In certain embodiments, the pharmaceutical composition is in a single unit dosage form. In certain embodiments, the single unit dosage form is between about 300 mg to about 1200 mg unit dosage form (such as about 300 mg, about 900 mg, and about 1200 mg) of a C5 inhibitor, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant. In certain embodiments, the pharmaceutical composition is lyophilized. In certain embodiments, the pharmaceutical composition is a sterile solution. In certain embodiments, the pharmaceutical composition is a preservative free formulation. In certain embodiments, the pharmaceutical composition comprises a 300 mg single-use formulation of 30 ml of a 10 mg/ml sterile, preservative free solution.

In certain embodiments, an anti-C5 full-length antibody (such as eculizumab or a variant thereof) is administered according to the following protocol: 600 mg via 25 to 45 minute IV infusion every 7+/−2 days for the first 4 weeks, followed by 900 mg for the fifth dose 7±2 days later, then 900 mg every 14±2 days thereafter. An anti-C5 antibody or polypeptide can be administered via IV infusion over 25 to 45 minutes. In another embodiment, an anti-C5 polypeptide full-length antibody is administered according to the following protocol: 900 mg via 25 to 45 minute IV infusion every 7+/−2 days for the first 4 weeks, followed by 1200 mg for the fifth dose 7±2 days later, then 1200 mg every 14±2 days thereafter. An anti-C5 antibody can be administered via IV infusion over 25 to 45 minutes. An exemplary pediatric dosing of, for example, an anti-C5 full-length antibody (such as eculizumab or a variant thereof), tied to body weight, is shown in Table 2:

TABLE 2
Exemplary dosing Recommendations in Pediatric
Subjects for Full-length Antibodies

| Subject Body Weight | Induction | Maintenance |
|---|---|---|
| 40 kg and over | 900 mg weekly × | 1200 mg at week 5; then 1200 |
| | 4 doses | mg every 2 weeks |
| 30 kg to less | 600 mg weekly × | 900 mg at week 3; then 900 |
| than 40 kg | 2 doses | mg every 2 weeks |
| 20 kg to less | 600 mg weekly × | 600 mg at week 3; then 600 |
| than 30 kg | 2 doses | mg every 2 weeks |
| 10 kg to less | 600 mg weekly × | 300 mg at week 2; then 300 |
| than 20 kg | 1 dose | mg every 2 weeks |
| 20 kg to less | 600 mg weekly × | 600 mg at week 2; then 600 |
| than 30 kg | 1 dose | mg every 3 weeks |

Note that in certain other embodiments the anti-C5 polypeptides that are not full-length antibodies and are smaller than a full-length antibody can be administered at a dosage that correspond to the same molarity as the dosage for a full-length antibody.

The aqueous solution can have a neutral pH, e.g., a pH between, e.g., about 6.5 and about 8 (e.g., between and inclusive of 7 and 8). The aqueous solution can have a pH of about any of the following: 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) about 6 (e.g., greater than or equal to about any of the following: 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than about pH 8.

In some embodiments, the C5 inhibitor, including a polypeptide inhibitor, is administered intravenously to the subject, including by intravenous injection or by intravenous infusion. In some embodiments, the anti-C5 antibody is administered intravenously to the subject, including by intravenous infusion. In some embodiments, the C5 inhibitor, including a polypeptide inhibitor, is administered to the lungs of the subject. In some embodiments, the C5 inhibitor, including a polypeptide inhibitor, is administered to the subject by subcutaneous injection. In some embodiments, the inhibitor, including a polypeptide inhibitor, is administered to the subject by way of intraarticular injection. In some embodiments, the C5 inhibitor, including a polypeptide inhibitor, is administered to the subject by way of intravitreal or intraocular injection. In some embodiments, the inhibitor, including a polypeptide inhibitor, is administered to the subject by pulmonary delivery, such as by intrapulmonary injection (especially for pulmonary sepsis). Additional suitable routes of administration are also contemplated.

A C5 inhibitor, such as a C5-binding polypeptide, can be administered to a subject as a monotherapy. In some embodiments, the methods described herein can include administering to the subject one or more additional treatments, such as one or more additional therapeutic agents.

The additional treatment can be any additional treatment, including experimental treatments, or a treatment for a symptom of an infectious disease, such as fever, etc. The other treatment can be any treatment, any therapeutic agent, that improves or stabilizes the subject's health. The additional therapeutic agent(s) includes IV fluids, such as water and/or saline, acetaminophen, heparin, one or more clotting factors, antibiotics, etc. The one or more additional therapeutic agents can be administered together with the C5 inhibitor as separate therapeutic compositions or one therapeutic composition can be formulated to include both: (i) one or more C5 inhibitors such as C5-binding polypeptides and (ii) one or more additional therapeutic agents. An additional therapeutic agent can be administered prior to, concurrently, or after administration of the C5-binding polypeptide. An additional agent and a C5 inhibitor, such as C5-binding polypeptide, can be administered using the same delivery method or route or using a different delivery method or route. The additional therapeutic agent can be another complement inhibitor, including another C5 inhibitor.

In some embodiments, an inhibitor, such as a C5-binding polypeptide, can be formulated with one or more additional active agents useful for treating a complement mediated disorder caused by an infectious agent in a subject.

When a C5 inhibitor is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of a C5 inhibitor and a sub-therapeutic amount of one or more additional active agents such that the components in total are therapeutically effective for treating a complement mediated disorder caused by an infectious agent. Methods for determining a therapeutically effective dose of an agent such as a therapeutic antibody are known in the art.

The compositions can be administered to a subject, e.g., a human subject, using a variety of methods that depend, in part, on the route of administration. The route can be, e.g., intravenous (“IV”) injection or infusion, subcutaneous (“SC”) injection, intraperitoneal (“IP”) injection, pulmonary delivery such as by intrapulmonary injection (especially for pulmonary sepsis), intraocular injection, intraarticular injection, intramuscular (“IM”) injection, or any other suitable route.

A suitable dose of a C5 inhibitor, including a C5-binding polypeptide, which dose is capable of treating or preventing a complement mediated disorder caused by an infectious agent in a subject, can depend on a variety of factors including, e.g., the age, gender, and weight of a subject to be treated and the particular inhibitor compound used. Other factors affecting the dose administered to the subject include, e.g., the type or severity of the complement mediated disorder caused by an infectious agent. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse).

A C5 inhibitor can be administered as a fixed dose, or in a milligram per kilogram (mg/kg) dose. In some embodiments, the dose can also be chosen to reduce or avoid production of antibodies or other host immune responses against one or more of the active antibodies in the composition.

A pharmaceutical composition can include a therapeutically effective amount of a C5 inhibitor. Such effective amounts can be readily determined by one of ordinary skill in the art.

In certain embodiments, the dosing of a C5 inhibitor, such as eculizumab or a variant thereof, can be as follows: (1) administering to a subject with a complement mediated disorder caused by an infectious agent about 900 milligrams (mg) of eculizumab each week for the first 3 weeks, or (2) 1200 milligrams (mg) of eculizumab each week for the first 3 weeks and (3) followed by an about 1200 mg dose on weeks 4, 6, and 8. After an initial 8-week eculizumab treatment period, the treating medical practitioner (such as a physician) can optionally request (and administer) treatment with eculizumab about 1200 mg every other week for an additional 8 weeks. The subject can then be observed for 28 weeks following eculizumab treatment.

While in no way intended to be limiting, exemplary methods of administration for a single chain antibody such as a single chain anti-C5 antibody (that inhibits cleavage of C5) are described in, e.g., Granger et al. (2003) Circulation 108:1184; Haverich et al. (2006) Ann Thorac Surg 82:486-492; and Testa et al. (2008) J Thorac Cardiovasc Surg 136(4):884-893.

The terms “therapeutically effective amount” or “therapeutically effective dose,” or similar terms used herein are intended to mean an amount of a C5 inhibitor, such as eculizumab or ravulizumab, an antigen-binding fragment thereof, an antigen-binding variant thereof, a polypeptide comprising the antigen-binding fragment of eculizumab or ravulizumab, a fusion protein comprising the antigen binding fragment of eculizumab, ravulizumab or variant thereof, or a single chain antibody version of eculizumab, ravulizumab, or variant thereof, that will elicit the desired biological or medical response.

In certain embodiments, for a subject with sepsis, a therapeutically effective amount of a C5 inhibitor can include an amount (or various amounts in the case of multiple administration) that improves the subject's chance of survival (by, e.g., any amount of time, such as one day or more), reduces C5a levels, reduces serum LDH levels, results in the subject having little to no organ failure, reduces levels of one or more of lactic acid, serum glutamic oxaloacetic transaminase (“SGOT”), creatine kinase, and creatine, reduces C-reactive protein level, reduces procalcitonin level, reduces serum amyloid A level, reduces mannan and/or antimannan antibody levels, reduces interferon-γ-inducible protein 10 (“IP-10”) level, increases levels of one or more of platelets and plasma bicarbonate level, decreases levels of one or more of the proinflammatory cytokines that are over-produced, or reduces other symptoms of the disease, or any combination thereof. All of these parameters can be ascertained or measured by known methods to a person skilled in the art.

In some embodiments, a composition described herein contains a therapeutically effective amount of a C5 inhibitor, such as a C5-binding polypeptide. In some embodiments, the composition contains any C5 inhibitor, such as a C5-binding polypeptide, and one or more (e.g., one, two, three, four, five, six, seven, eight, nine, ten, or eleven or more) additional therapeutic agents to treat or prevent a complement mediated disorder caused by an infectious agent, such that the composition as a whole is therapeutically effective. For example, a composition can contain a C5-binding polypeptide described herein and an immunosuppressive agent, wherein the polypeptide and agent are each at a concentration that when combined are therapeutically effective for treating or preventing a complement mediated disorder caused by an infectious agent in a subject.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries (e.g., PUBMED, NCBI or UNIPROT accession numbers), and other references mentioned herein are incorporated by reference in their entirety. In case of conflict, the present specification, including definitions, will control.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

The following examples are merely illustrative and should not be construed as limiting the scope of this disclosure in any way as many variations and equivalents will become apparent to those skilled in the art upon reading the present disclosure.

### Example 1: Second Nested Trial: Efficacy of Eculizumab for Patients with COVID-19—“ECU-COVID Trial”

A nested clinical trial is conducted to test the efficacy of eculizumab (SOLIRIS®) in patients with COVID-19. The eculizumab dosage regimen for the treatment of participants with SARS-CoV-2 infection with a clinical presentation consistent with COVID-19 severe pneumonia, acute lung injury, or ARDS COVID-19 is based on the induction dosage regimen approved for adult patients with atypical hemolytic uremic syndrome, generalized myasthenia gravis, and neuromyelitis optica spectrum disorder.

SOLIRIS® administered intravenously at a dose of 900 mg on Days 1, 8, 15 and 22. Based on the monitoring of eculizumab Plasma Level and free C5 free C-5, CH50 suppression, Supplemental dosing of 900 mg can be administered at Day 4, Day 12, and Day 18. Complement monitoring (weekly prior enrolment) during the treatment period and post treatment period, includes: CH50, C3, C4, C4d, sC5b9, C5 and residual eculizumab plasma level before each SOLIRIS® administration and at Day 1, Day 2, Day 3, and Day 6 to ensure satisfactory drug exposition.

Participants who have not received a meningococcal vaccination within the past 5 years may be unable to receive meningococcal vaccinations prior to initiating treatment with SOLIRIS® in this study. If vaccination cannot be confirmed, the participant receives prophylactic antibiotics against meningococcal infection prior to initiating SOLIRIS® treatment and for at least 3 months from the last infusion of SOLIRIS®.

When participants can be vaccinated, vaccines against meningococcal serotypes A, C, Y, W135, and B, where available, are recommended to prevent common pathogenic meningococcal serotypes. Participants must be vaccinated or revaccinated according to current national vaccination guidelines or local practice for vaccination use with complement inhibitors (e.g., SOLIRIS®). Vaccination may not be sufficient to prevent meningococcal infection. Consideration should be given per official guidance and local practice on the appropriate use of antibacterial agents.

1. Inclusion and Exclusion Criteria

To be included in the trial, patients must meet the following criteria:
1. Be a patient included in the CORIMUNO-19 cohort;
2. Belong to one of the 2 following groups:
   Group 1: 60 patients not requiring ICU at admission with moderate and severe pneumopathy according to the WHO Criteria of severity of COVID pneumopathy, meeting all of the 3 following criteria: age 18-70 years, positive PCR SARS-CoV-2, and severe pneumonia requiring ≥5 L/min of oxygen to maintain SpO2 levels, e.g., >97%; or
   Group 2: 60 patients requiring ICU based on Criteria of severity of COVID pneumopathy: respiratory failure and requiring mechanical ventilation, renal injury defined by AKI>2 or requiring dialysis, vasopressive support and/or do-not-resuscitate order (DNR order);
3. Vaccinated against meningococcal infections within 3 years prior to, or at the time of, initiating SOLIRIS to reduce the risk of meningococcal infection (N. meningitidis) [(Bexsero® (2 injections with a minimum of 1 month interval) plus Menveo® or Niminrex®) and daily antibiotics (Oracilline®)]. It is anticipated that participants may be unable to receive meningococcal vaccinations prior to initiating treatment with SOLIRIS. If vaccination cannot be confirmed or if the patient cannot receive it, the participant receives prophylactic antibiotics against meningococcal infection prior to initiating SOLIRIS treatment and for at least 3 months from the last infusion of SOLIRIS. If the patient is vaccinated during the study, the patient must receive prophylactic antibiotics for at least two weeks after vaccination;
4. Female patients of childbearing potential and male patients with female partners of childbearing potential must follow protocol-specified guidance for avoiding pregnancy while on treatment and for 8 months after last dose of SOLIRIS®;
5. Body weight≥40 kg to <100 kg. Patients who are >100 kg can be enrolled if CH50 and drug levels can be checked regularly; and
6. Patient and/or Patient's legal guardian must be willing and able to give written informed consent according to the applicable regulations including emergency and intensive care contexts.

Patients are excluded from the trial if they meet any of the following criteria:
^{1.} Patients with exclusion criteria to the CORIMUNO-19 cohort;
^{2.} Pregnancy or lactation;
^{3.} History of N. meningitidis infection;
^{4.} Ongoing sepsis, presence or suspicion of active and untreated systemic bacterial infection prior study screening and untreated with antibiotics; or
^{5.} Hypersensitivity to any ingredient contained in SOLIRIS, including hypersensitivity to murine proteins.

1. Study Drug

SOLIRIS® is supplied in single 30 mL vials as a solution concentration of 10 mg/mL. Each vial contains 300 mg of SOLIRIS® for intravenous (IV) administration. SOLIRIS® is individually packaged in kits.

SOLIRIS® vials stored in a fridge between 2° C. to 8° C. in the original carton to protect from light until time of use. SOLIRIS® vials can also be stored in the original carton at controlled room temperature (not more than 25° C.) for only a single period up to 3 days. SOLIRIS® is not to be used beyond the expiration date stamped on the carton.

2. Endpoints

A core set of clinical measures is recorded daily the first 2 weeks and then every week. The core measures include measures of OMS progression scale, oxygenation, mechanical ventilation. For patients who are eligible for an intervention trial (in both the intervention and control arms), this day measurement includes trial-specific measures related to the trial outcomes of interest.

For the group 1 of patients not requiring ICU, the primary endpoint is Survival without needs of intubation at day 14. Thus, events considered are intubation or death. Secondary endpoints are:
1) OMS progression scale≤5 at day 4, defined as set forth in Table 3;
2) OMS progression scale at 4, 7 and 14 days;
3) Overall survival at 14, 28 and 90 days;
4) Time to discharge;
5) Time to oxygen supply independency;
6) Time to negative viral excretion; and
7) Biological parameters improvement (C5b9, estimated GFR, CRP, myoglobin, CPK, cardiac troponin, ferritin, lactate, cell blood count, liver enzymes, LDH, D-Dimer, albumin, fibrinogen, triglycerides, coagulation tests, urine electrolyte, creatinuria, proteinuria, uricemia, IL6, procalcitonin, immunophenotype and exploratory tests.

TABLE 3
OMS Progression Scale

| OMS Progression scale | Descriptor | Score |
|---|---|---|
| Uninfected | Uninfected; nonviral RNA | 0 |
| | detected | |
| Ambulatory | Asymptomatic; viral RNA | 1 |
| | detected | |
| Ambulatory | Symptomatic; Independent | 2 |
| Ambulatory | Symptomatic; Assistance needed | 3 |
| Hospitalized: mild disease | Hospitalized; No oxygen therapy | 4 |
| Hospitalized: mild disease | Hospitalized; oxygen by mask or | 5 |
| | nasal prongs | |
| Hospitalized: severe disease | Hospitalized; oxygen by NIV or | 6 |
| | High flow | |
| Hospitalized: severe disease | Intubation and Mechanical | 7 |
| | ventilation, pO2/FIO2 >=150 OR | |
| | SpO2/FIO2 >=200 | |
| Hospitalized: severe disease | Mechanical ventilation, | 8 |
| | (pO2/FIO2 <150 OR | |
| | SpO2/FIO2 <200) OR | |
| | vasopressors (norepinephrine >0.3 | |
| | microg/kg/min) | |
| Hospitalized: severe disease | Mechanical ventilation, | 9 |
| | pO2/FIO2 <150 AND | |
| | vasopressors (norepinephrine >0.3 | |
| | microg/kg/min), OR | |
| | Dialysis OR ECMO | |
| Death | Dead | 10 |

For the group 2 of patients requiring ICU, the primary endpoint is the decrease in organ failure at Day 3, defined by the relative variation in Sequential Organ Failure Assessment (SOFA) score at Day 3. Secondary endpoints include:
1. Secondary infections (pneumonia acquired);
2. Vasopressor free survival;
3. Ventilator free survival;
4. Overall survival in ICU and hospital;
5. Incidence of dialysis;
6. OMS progression scale at 4, 7 and 14 days, overall survival at 14, 28 and 90 days, the 28-day ventilator free-days, the evolution of PaO2/FiO2 ratio, respiratory acidosis at day 4 (arterial blood pH of <7.25 with a partial pressure of arterial carbon dioxide [Paco2] of ≥60 mm Hg for >6 hours), time to oxygen supply independency, duration of hospitalization, time to negative viral excretion, time to ICU and hospital discharge; and
7. Biological parameters improvement: sC5b9, estimated GFR, CRP, cardiac troponin, urine electrolyte and creatinine, proteinuria, uricemia, IL6, myoglobin, KIM-1, NGAL, CPK, ferritin, lactate, cell blood count, liver enzymes, LDH, D-Dimer, albumin, fibrinogen, triglycerides, coagulation tests (including activated partial thromboplastin time), procalcitonin, immunophenotype, exploratory tests, rate of renal replacement therapy, and ventilation parameters.

In the setting of COVID-19 NCP and short-term immunomodulatory therapy, the following major safety endpoints are monitored: blood cells and platelets counts and liver transaminases, frequently, every three days systematically.

The clinical benefit is globally to prevent death in all patient groups. Other benefits are to: (1) blunt not only the pneumopathy-induced damage, but also other COVID-19-associated injuries such as acute kidney injury (AKI), myocarditis, secondary bacterial infections, (2) shorten the duration of hospital stay with minimization of physical (hospital acquired pressure ulcers, increased morbidity and mortality associated with nosocomial infections), psychological and economic complications related with prolonged stay, (3) shorten the hospital stay fosters not only individual clinical benefit, but also collective clinical benefit through facilitation of collective access to caregivers, and (4) limit long term sequelae, in particular, lung fibrosis and chronic kidney disease secondary to acute kidney injury (markedly prevalent in about 20% of individuals with ARDS).

3. Statistical Methods

In the non-ICU group, the primary endpoint is Survival without needs of intubation at day 14. Given preliminary data, the expected rate in the control arm is 50%. A two-sided logrank test with an overall sample size of 60 subjects (30 in the control group and 30 in the treatment group) achieves 80.4% power at a 0.05 significance level to detect a survival free of IOT of 75% (that is, a hazard ratio of 0.415) when the proportion surviving in the control group is 0.50. The study lasts for 60 time periods of which subject accrual (entry) occurs in the first 40 time periods. The accrual pattern across time periods is uniform (all periods equal). No subjects drop out or switch.

In the ICU group, the primary endpoint is diminution of organ failure at Day 3, defined by the relative variation in SOFA score at day 3. Group sample sizes of 29 and 29 achieve 80.141% power to reject the null hypothesis of equal means when the population mean difference is μ1−μ2=0 versus 1.5 with a standard deviation for both groups of 2.0 and with a significance level (alpha) of 0.050 using a two-sided two-sample equal-variance t-test.

Analysis is based on the intent to treat principle. Analyses of the censored data use the Kaplan Meier estimates, then compared by the logrank test. Analyses of the variation in SOFA scores are based on the Wilcoxon rank sum test, assuming death as a maximal SOFA score value at day 14. Interim analyses use Bayesian monitoring in order to avoid inflation of type I error. All statistical analyses are performed using R software (R Foundation for Statistical Computing, Vienna, Austria. http://www.r-project.org/) v. 3.6 or later, or SAS software v 9.1.

### Example 2: Modified Regimen

The protocol of Example 1 is incorporated by reference except for a minor modification in the following respect—SOLIRIS (intravenous) dosing for expanded access program (EAP): Day 1: 1200 mg, Day 4: 1200 mg, Day 8: 1200 mg, Day 12: Optional dose of 900 mg or 1200 mg if indicated based on therapeutic dose monitoring (TDM), Day 15: 900 mg, Day 18: Optional dose of 900 mg or 1200 mg if indicated based on TDM, Day 22: 900 mg.

As provided herein, SOLIRIS® is administered intravenously at a dose of 1200 mg on Days 1, 4, and 8. Based on the TDM, e.g., monitoring of Eculizumab Plasma Level and free C5 free C-5, CH50 suppression, optional dosing of 900 mg or 1200 mg could be administered at D12. Next, 900 mg dose is administered intravenously on D15 and based on the TDM, e.g., as provided above, optional dosing of 900 mg or 1200 mg could be administered at D18. Finally, 900 mg dose is administered intravenously on D22.

Complement monitoring, weekly prior enrolment, during the treatment period and post treatment period, includes: CH50, C3, C4, C4d, sC5b9, C5 and residual Eculizumab plasma level before each SOLIRIS administration and at various time points (e.g., D4, D8, D12, D15, D18, and D22) to ensure satisfactory drug exposition.

### Example 3: SOLIRIS® Treatment of Participants with COVID-19—an Expanded Access Program for Hospital-Based Emergency Treatment

A clinical trial is conducted to assess the efficacy of SOLIRIS® (eculizumab) in treating participants with Coronavirus Disease 2019 (COVID-19) (NCT04355494; Apr. 21, 2020).

1. Objectives

The primary objective is to assess survival in participants with COVID-19 receiving SOLIRIS® treatment (e.g., as assessed by survival (based on all-cause mortality) at Day 15).

The secondary objective is to assess evidence of efficacy of SOLIRIS® in participants with COVID-19 (e.g., as assessed by (1) number of days alive and free of mechanical ventilation at Day 15 and Day 29, (2) improvement of oxygenation from Day 1 to Day 15 and Day 29, (3) number of days alive and free of supplemental oxygen at Day 15 and Day 29, (4), duration of intensive care unit stay, and (5) duration of hospitalization).

The safety objective is to characterize the overall safety of SOLIRIS® in the treatment of COVID-19 (e.g., as assessed by incidence of treatment emergent serious adverse events).

The exploratory objective is to assess the longer-term effect of SOLIRIS® treatment on survival (e.g., as assessed by survival (based on all-cause mortality) at Day 29).

Pharmacokinetic/Pharmacodynamic/Immunogenicity objectives include: (1) evaluating the PK/PD of eculizumab in participants with COVID-19 (e.g., as assessed by (a) change in serum eculizumab concentration over time, (b) change in pharmacodynamic markers over time (including but not limited to CH50, C5b9, other complement proteins), and (c) presence of anti-drug antibodies to eculizumab) and (2) determining the effect of C5 inhibition on systemic activation of complement and inflammation (e.g., as assessed by change in absolute levels of soluble biomarkers associated with complement activation and inflammatory processes).

2. Overall Design

This is an open-label, multicenter Expanded Access Program (EAP) that is intended to allow participants with a confirmed diagnosis of SARS-CoV-2 infection with a clinical presentation consistent with COVID-19 severe pneumonia, acute lung injury, or ARDS to have access to SOLIRIS®. Participants admitted to a designated hospital facility who qualify for emergency treatment are offered the opportunity to be treated with up to 4 infusions of SOLIRIS®.

The EAP consists of a Screening Period of up to 7 days, a Treatment Period from 2 to up to 5 weeks, a final in-hospital assessment on day of discharge or at Day 29, whichever occurs first, and 3 monthly safety follow-up telephone calls. Screening and the Day 1 visits can occur on the same day if the participant meets all of the inclusion and none of the exclusion criteria.

100 participants are to be enrolled to receive SOLIRIS®. For each participant, the total duration of the program is anticipated to be up to 4.5 months and consists of the following: (a) approximately 5 weeks while the participant is hospitalized (up to 1 week for Screening, up to 4 weeks for treatment and a final assessment at Day 29 or on day of discharge, whichever occurs first) and (b) three additional safety follow-up telephone calls, conducted once a month.

3. SOLIRIS® Dose and Dosage Regimen:

The proposed SOLIRIS® dosage regimen for the treatment of participants with SARS-CoV-2 infection with a clinical presentation consistent with COVID-19 severe pneumonia, acute lung injury, or ARDS COVID-19 is based upon examination of preliminary serum free eculizumab concentrations, CH50 and serum C5b9 levels in patients with COVID-19 (unpublished data). These data suggest that the complement system is amplified beyond that observed in patients with aHUS, necessitating increased and more frequent dosing of SOLIRIS than what is currently approved for the treatment of patients with aHUS to achieve complete and sustained complement inhibition.

To account for SARs-CoV2-related amplification of complement, SOLIRIS® is administered intravenously at a dose of 1200 mg on Days 1, 4, and 8 and 900 mg on Days 15 and 22. Optional doses of 900 or 1200 mg can be administered on Days 12 and 18, per Investigator decision in consultation with the Medical Monitor. A further change is that weight is now required only at screening and Dosing Day 1, as dosing is fixed Optional additional endpoint of presence of anti-drug antibodies.

4. Schedule of Activities

The schedule of activities is set forth in Table 4.

TABLE 4
Schedule of Activities

| | Period | | | | | | | | | |
|---|---|---|---|---|---|---|---|---|---|---|
| | | | | | | | | | | Safety |
| | | | | | | | | | | Follow |
| | Screening | Treatment Period | | | | | | | | Up³ |
| | EAP Day (D) | | | | | | | | | |
| | D −7 | | | | | | | | D29 | |
| | to D −1 | D1¹ | D4 | D8 | D12² | D15 | D18² | D22 | or ET | |
| | EAP Window | | | | | | | | | |
| | N/A | N/A | ±1 | ±1 | ±1 | ±1 | ±1 | ±1 | ±1 | +7 |
| Eligibility | | | | | | | | | | |
| Informed consent | X | | | | | | | | | |
| Inclusion/exclusion | X | | | | | | | | | |
| Confirmation of vaccination or | X | | | | | | | | | |
| appropriate prophylaxis against | | | | | | | | | | |
| N. meningitidis⁴ | | | | | | | | | | |
| Medical history | X | | | | | | | | | |
| Demographics | X | | | | | | | | | |
| Screening Laboratory Tests | | | | | | | | | | |
| Confirmed positive SARS- | X | | | | | | | | | |
| CoV-2 test | | | | | | | | | | |
| Chest CT or X-ray⁵ | X | | | | | | | | | |
| Pregnancy test⁶ | X | | | | | | | | X | |
| Administration of SOLIRIS | | | | | | | | | | |
| SOLIRIS(mg) | | 1200 | 1200 | 1200 | X⁷ | 900 | X⁷ | 900 | | |
| Efficacy Assessments | | | | | | | | | | |
| Survival status | | ← | | | | | | | | X |
| Mechanical ventilation status | | ← | | | | | | | | |
| Supplemental oxygenation status | | ← | | | | | | | | |
| Hospitalization status | | ← | | | | | | | | |
| SpO2 and/or PaO2⁸ | | ← | | | | | | | | |
| Safety Assessments | | | | | | | | | | |
| Physical examination | X | X | X | X | X | X | X | X | X | |
| Body weight | X | X | | | | | | | | |
| Vital sign measurements⁹ | X | X | X | X | X | X | X | X | X | |
| Serious adverse event review | | ← | | | | | | | | X |
| and evaluation | | | | | | | | | | |
| Review safety card¹⁰ | | X | | | | | | | | |
| Safety Laboratory Tests¹¹ | | | | | | | | | | |
| Clinical chemistry | X | X | X | X | X | X | X | X | X | |
| Hematology | X | X | X | X | X | X | X | X | X | |
| Optional/Exploratory | | | | | | | | | | |
| Assessments | | | | | | | | | | |
| PK and PD sampling¹² | | X | X | X | X | X | X | X | X | |
| Serum and plasma biomarkers¹³ | | X | X | X | X | X | X | X | X | |
| Antidrug antibodies¹⁴ | | X | | | | | | | X | |
| Other | | | | | | | | | | |

| | |
|---|---|
| Concomitant medication¹⁵ | ← |

¹The Day 1 visit can occur on the same day as Screening.
²OPTIONAL. Assessments on Days 12 and 18 are performed only if the participant receives a dose of SOLIRIS ® on that day.
³A safety follow-up telephone call is conducted once a month for 3 months following the participant's last dose of SOLIRIS ® to review participant status, including survival and pregnancy, and to obtain information about new or worsening TESAEs. The follow up is conducted as a telephone call if the participant has been discharged from the hospital or an in-person visit if the participant is still hospitalized.
⁴Confirmation of meningococcal vaccination within the past 5 years prior to initiating SOLIRIS ® treatment. If vaccination cannot be confirmed, the participant should receive prophylactic antibiotics prior to initiating SOLIRIS ® treatment and for at least 3 months from the last infusion of SOLIRIS ®.
⁵Can be performed at Screening or within the 7 days prior to Screening.
⁶Urine pregnancy tests to be performed in female participants of childbearing potential only. A positive urine test result is confirmed with a serum pregnancy test.
⁷OPTIONAL. Doses of 900 or 1200 mg are administered on Days 12 and 18, per Investigator decision in consultation with the Medical Monitor.
⁸SpO2 is measured by pulse oximetry. PaO2 is measured by arterial blood gas. The highest daily measurement on the lowest inspired supplemental oxygen level is recorded in the eCRF.
⁹Vital sign measurements are taken after the participant has been resting for at least 5 minutes and will include systolic and diastolic BP (millimeters of mercury [mm Hg]), heart rate (beats/minute), respiratory rate (breaths/minute), and temperature (degrees Celsius [° C.] or degrees Fahrenheit [° F.]). On dosing days, vital signs are taken predose.
¹⁰Review the Participant Safety Information Card with the participant (including discussion of the risks associated with SOLIRIS ® treatment, such as meningococcal infection) at the time of dosing and discharge. Upon discharge, the participant must carry the Participant Safety Information Card at all times and for at least 3 months after their last infusion of SOLIRIS.
¹¹Clinical safety laboratory measurements is collected predose on dosing days.
¹²OPTIONAL. Serum samples for PK and PD analyses (including but not limited to CH50, C5b9, other complement proteins) are collected at the indicated visits and stored at the EAP site, prior to analysis. Samples are collected predose (any time before infusion start) and any time after end-of-infusion. Postdose samples must be collected from a separate line or needle stick, not from the infusion line.
¹³OPTIONAL. Serum samples for biomarker analyses are collected at the indicated visits and stored at the EAP site, prior to analysis. Samples are collected predose (any time before infusion start).
¹⁴OPTIONAL. Serum samples for antidrug antibodies are collected pre-dose on Day 1 and at Day 29 or ET and stored at the EAP site prior to analysis.
¹⁵Concomitant medications considered relevant to the treatment of COVID-19 or SOLIRIS ® treatment (e.g., antimicrobials, antivirals, steroids, IVIg, investigational agents) that the participant is receiving at the time of enrollment or receives during the EAP are recorded on the eCRF.
Abbreviations: BP = blood pressure; C = complement component/protein; COVID-19 = Coronavirus Disease 2019; CT = computed tomography; EAP = Expanded Access Program; eCRF = electronic case report form; ET = early termination; IVig = intravenous immunoglobulin; N/A = not applicable; PaO2 = partial pressure of oxygen; PK = pharmacokinetics; SAE = serious adverse event; SpO2 = peripheral capillary oxygen saturation TESAE = treatment-emergent serious adverse event.

5. Inclusion and Exclusion Criteria

Participants are eligible to be included in the EAP only if all of the following criteria apply:
1. Males or females≥18 years of age and ≥40 kg at the time of providing informed consent;
2. Confirmed diagnosis of SARS-CoV-2 infection presenting as severe COVID-19 requiring hospitalization;
3. Symptomatic, bilateral pulmonary infiltrates confirmed by CT or X-ray at Screening or within the 7 days prior to Screening;
4. Severe pneumonia, acute lung injury, or ARDS requiring oxygen supplementation (WHO 2020); and
6. Informed consent is obtained. If allowable per local regulations, a participant's Legally Acceptable Representative (LAR) can provide consent if a participant is unable to do so. Where applicable and allowed by local regulations, and following IRB/EC approval, participants who are unable to provide informed consent and whose LAR is unavailable can be enrolled per the decision of the Principal Investigator or designee. The patient or, where appropriate, Legally Acceptable Representative, should be informed and their consent to remain in the study should be requested as soon as feasible.

Participants are excluded from the EAP if any of the following criteria apply:
Confirmed diagnosis of SARS-CoV-2 infection presenting as mild to moderate COVID-19, even if the participant is hospitalized;
7. Participant is not expected to survive more than 24 hours;
8. Participant has an unresolved Neisseria meningitidis infection; or
9. Hypersensitivity to murine proteins or to one of the excipients of SOLIRIS®.

6. SOLIRIS®

SOLIRIS® is a humanized monoclonal antibody that was derived from the murine anti-human C5 antibody m5G1.1. Eculizumab specifically binds C5, thereby inhibiting its cleavage to C5a and C5b during complement activation. This strategic blockade of the complement cascade at C5 prevents the release of pro-inflammatory mediators and the formation of the cytolytic pore, while preserving the early components of complement activation that are essential for the opsonization of microorganisms and clearance of immune complexes.

The SOLIRIS® dosage regimen for the treatment of EAP participants is 1200 mg administered by IV infusion on Days 1, 4 and 8, and 900 mg on Days 15 and 22. At the Investigator or designee's discretion, additional doses of 900 or 1200 mg can be administered on Days 12 and 18.

SOLIRIS® is only be administered via IV infusion via gravity feed, a syringe-type pump, or an infusion pump, and must be diluted to a final concentration of 5 mg/mL prior to administration. The diluted SOLIRIS is IV administered over approximately 35 minutes. Diluted SOLIRIS is stable for 24 hours between 2° C. to 8° C. (36° F. to 46° F.) and at room temperature. Participants are monitored for at least 1 hour following the end-of-infusion for signs or symptoms of an infusion-associated reaction. If an infusion-associated reaction occurs during the administration of SOLIRIS®, the infusion can be slowed or stopped at the discretion of the Investigator or designee, depending upon the nature and severity of the event.

SOLIRIS® is manufactured and supplied in single 30 mL vials as a solution concentration of 10 mg/mL (Table 5). Each vial contains 300 mg of SOLIRIS® for IV administration. SOLIRIS® is individually packaged in kits. SOLIRIS® orders are released to each site upon receipt of all required documents based upon applicable regulations.

TABLE 5
SOLIRIS ® Dosage Form and Strength

| | |
|---|---|
| Product Name | SOLIRIS |
| Dosage Form | Concentrate for solution for infusion |
| Unit Dose | 300 mg |
| Route of Administration | Intravenous infusion |
| Physical Description | 30 mL vial of 10 mg/mL sterile, preservative |
| | free |
| Manufacturer | Alexion Pharmaceuticals, Inc. |

SOLIRIS® vials are stored refrigerated between 2° C. to 8° C. (36° F. to 46° F.) in the original carton to protect from light until time of use. SOLIRIS® vials can also be stored in the original carton at controlled room temperature (not more than 25° C. or 77° F.) for only a single period up to 3 days. SOLIRIS® is not used beyond the expiration date stamped on the carton. SOLIRIS® is not frozen or shaken. The SOLIRIS® Package Insert contains information on the stability and storage of diluted solutions of SOLIRIS®.

7. Concomitant Therapy

Concomitant medications considered relevant to treatment of COVID-19 or SOLIRIS® treatment (e.g., antimicrobials, antivirals, steroids, IVIg, investigational agents) that the participant is receiving at the time of enrollment or receives during the EAP must be recorded along with (1) reason for use, (2) dates of administration, including start and end dates, and (3) dosage information including dose and frequency.

8. Efficacy Assessments

The primary efficacy assessment is survival at Day 15. The following secondary efficacy-related parameters are also be measured throughout the EAP: mechanical ventilation status, oxygen saturation levels (SpO2 and/or PaO2), supplemental oxygen status, time in the intensive care unit, and duration of hospitalization.

Exploratory assessments include: (1) survival at Day 29, (2) change in serum eculizumab concentration over time, (3) change in free serum C5 concentration over time, and (4) change in absolute levels of soluble biomarkers associated with complement activation and inflammatory processes over time.

Planned timepoints for all safety assessments are provided in Table 4.

Physical examinations include, at a minimum, assessments of the cardiovascular, respiratory, gastrointestinal and neurological systems. Height and weight (at Screening only) are also measured and recorded. Vital signs measured include temperature, systolic and diastolic blood pressure, heart rate, and respiratory rate.

Table 6 provides a list of clinical laboratory tests. Table 4 sets forth the timing and frequency of assessments.

TABLE 6
Protocol-Required Laboratory Assessments

| Laboratory | |
|---|---|
| Assessments | Parameters |
| Hematology | Platelet count |
| | Red blood cell count¹ |
| | Hemoglobin |
| | Hematocrit |
| | White blood cell count with differential (neutrophils, |
| | lymphocytes, monocytes¹, eosinophils¹, basophils¹) |
| Clinical | Alanine aminotransferase/serum glutamic-pyruvic |
| | transaminase |
| | Aspartate aminotransferase/serum glutamic-oxaloacetic |
| | transaminase |
| | Blood urea nitrogen |
| | C-reactive protein |
| chemistry | Creatinine |
| | Glucose (nonfasting)¹ |
| | Total and direct bilirubin¹ |
| | Total protein¹ |
| Other | 1 Arterial blood gas |
| laboratory | 2 Highly sensitive serum human chorionic gonadotropin |
| tests | pregnancy test (as needed for women of childbearing |
| | potential)² |

Denotes parameter is collected, but not recorded in the eCRF.
Local urine testing is standard for the protocol, unless serum testing is required by local regulation or Independent Ethics Committee/ Institutional Review Board.

9. Vaccinations and Prophylactic Antibiotics

Participants who have not received a meningococcal vaccination within the past 5 years may be unable to receive meningococcal vaccinations prior to initiating treatment with SOLIRIS® in this EAP. If vaccination cannot be confirmed, the participant receives prophylactic antibiotics against meningococcal infection prior to initiating SOLIRIS® treatment and for at least 3 months from the last infusion of SOLIRIS®.

When participants can be vaccinated, vaccines against meningococcal serotypes A, C, Y, W135, and B, where available, are recommended to prevent common pathogenic meningococcal serotypes. Participants must be vaccinated or revaccinated according to current national vaccination guidelines or local practice for vaccination use with complement inhibitors (e.g., SOLIRIS). Vaccination may not be sufficient to prevent meningococcal infection. Consideration should be given per official guidance and local practice on the appropriate use of antibacterial agents.

10. Serious Adverse Events

Serious adverse events (SAEs) are defined in Table 7. All SAEs are reported to the Investigator or qualified designee by the participant (or, when appropriate, by a caregiver, surrogate, or the participant's Legally Authorized Representative).

TABLE 7
Serious Adverse Event Definition
An SAE is defined as any untoward medical occurrence that, at any dose:

Results in death
Is life-threatening
The term “life-threatening” in the definition of “serious” refers to an event in which the patient
was at risk of death at the time of the event. It does not refer to an event, which hypothetically
might have caused death, if it was more severe.
Requires inpatient hospitalization or prolongation of existing hospitalization
In general, hospitalization signifies that the patient has been detained (usually involving at
least an overnight stay) at the hospital or emergency ward for observation and/or treatment that
would not have been appropriate in the physician's office or outpatient setting. Complications
that occur during hospitalization are AEs. If a complication prolongs hospitalization or fulfills
any other serious criteria, the event is serious. When in doubt as to whether “hospitalization”
occurred or was necessary, the AE should be considered serious.
Hospitalization for elective treatment of a pre-existing condition that did not worsen from
baseline is not considered an AE.
Results in persistent disability/incapacity
The term disability means a substantial disruption of a person's ability to conduct normal
life functions.
This definition is not intended to include experiences of relatively minor medical
significance such as uncomplicated headache, nausea, vomiting, diarrhea, influenza, and
accidental trauma (eg, sprained ankle) which may interfere with or prevent everyday life
functions but do not constitute a substantial disruption.
Is a congenital anomaly/birth defect
Medical or scientific judgment should be exercised in deciding whether SAE reporting is
appropriate in other situations such as important medical events that may not be
immediately life-threatening or result in death or hospitalization but may jeopardize the
patient or may require medical or surgical intervention to prevent one of the other
outcomes listed in the above definition. These events should usually be considered
serious.
Examples of such events include invasive or malignant cancers, intensive treatment in an
emergency room or at home for allergic bronchospasm, blood dyscrasias or convulsions
that do not result in hospitalization, or development of drug dependency or drug abuse.

11. Pharmacokinetics

Samples can be collected to determine serum concentrations of SOLIRIS®. The actual date and time (24-hour clock time) of each sample is recorded.

12. Pharmacodynamics

Samples can be collected to assess the effect of SOLIRIS® on PD markers (including but not limited to CH50, C5b9, or other complement proteins). The actual date and time (24-hour clock time) of each sample are recorded.

13. Immunogenicity

Serum samples macany be collected to evaluate the presence or development of antidrug antibodies to eculizumab. Samples are collected as noted in the Schedule of Assessments.

14. Biomarkers

Samples can be collected for evaluation of complement pathway proteins (e.g., sC5b-9, C5a, C3a, total C3, Factor B, and Factor Ba) and inflammatory cytokines (e.g., IL-1, IL-6, IL-8, IL-21, tumor necrosis factor [TNF]-b, and monocyte chemoattractant protein [MCP]-1) and their association with observed clinical responses to SOLIRIS.

15. Statistical Considerations

The purpose of this EAP is to provide SOLIRIS® as an emergency therapy for the treatment of participants with severe pneumonia, acute lung injury, or ARDS associated with SARS-CoV-2 infection; thus, there are no statistical considerations for the sample size. The population sets used for analysis are defined in Table 8.

TABLE 8
Analysis Populations

| Analysis Set | Definition | |
|---|---|---|
| Full Analysis | Includes all participants who enroll and who receive at least 1 dose of | |
| Set (FAS) | SOLIRIS ®. The FAS is used for the analysis of efficacy data and is | |
| | considered the primary analysis population. | |
| Per-Protocol | All participants in FAS who: | |
| Set (PPS) | 1. | Receive at least 2 doses of SOLIRIS ®. An exception is made for |
| | | participants who receive less than 2 doses of SOLIRIS ® due to |
| | | death; these participants are included in the PPS and counted as |
| | | failures for the primary endpoint |
| | 2. | Meet all inclusion and no exclusion criteria |
| | | The PPS is used for sensitivity analyses of the primary and secondary |
| | | efficacy endpoints. |

| | |
|---|---|
| Safety Set | The Safety Set is identical to the FAS and consists of all participants |
| | enrolled and who receive at least 1 dose of SOLIRIS ®. The Safety Set |
| | is used for the analysis of safety data. |

Summary statistics are presented overall and by visit, where applicable. Descriptive statistics for continuous variables include the number of participants, mean, standard deviation, median, 25th percentile, 75th percentile, minimum, and maximum. For categorical variables, frequencies, and percentages will be presented. Graphical displays are provided as appropriate. Any statistical analyses are performed based on a 2-sided Type I error of 5%. Analyses are performed using the SAS® software Version 9.4 or higher.

The primary efficacy endpoint is survival (based on all-cause mortality) at Day 15 and is summarized using the method of Kaplan and Meier (KM). The time at risk begins at the first dose of SOLIRIS® (Day 1). A censoring indicator is equal to 1 if the participant survived over this time, and 0 if the participant did not survive. Kaplan-Meier survival estimates and confidence intervals (95%) based on the complementary log-log transformation are presented at Day 15 and at Day 29. A Kaplan-Meier curve is produced.

The number of days alive and free of mechanical ventilation at Day 15 are summarized for all participants. If a participant is discharged from the hospital prior to Day 15, he/she is considered alive and free of mechanical ventilation. The number of days alive and free of mechanical ventilation at Day 29 is also be summarized.

Improvement of oxygenation is summarized using changes in SpO2 and PaO2 from Day 1 to Day 15 and Day 29. These re summarized for all patients and for patients without mortality.

The number of days alive and free of supplemental oxygen at Day 15 are summarized for all participants. If a participant is discharged from the hospital prior to Day 15, he/she is considered alive and free of supplemental oxygen. The number of days alive and free of supplemental oxygen at Day 29 is also summarized.

Duration of intensive care unit stay and hospitalization is summarized for all participants and for participants without mortality.

All safety analyses are made on the Safety Set.

The analysis and reporting of SAEs is based on TESAEs, defined as SAEs with onset on or after the first dose of SOLIRIS®. The incidence of TESAEs is summarized by System Organ Class (SOC) and Preferred Term, with additional summaries showing relationship to SOLIRIS®, TESAEs leading to SOLIRIS® discontinuation, and TESAEs resulting in death.

Laboratory measurements, as well as their changes from baseline, at each visit and shift from baseline, if applicable, are summarized. Vital sign measurements and physical examination findings are also summarized over time.

16. Pharmacokinetic/Pharmacodynamic/Biomarker Analyses

Collection of blood samples for exploratory analyses is optional. Blood samples can be collected at the timepoints indicated in the Schedule of Assessments and stored for PK and PD analyses. Serum samples can be collected at Screening and following treatment, at the timepoints indicated in the Schedule of Assessments for exploratory biomarker analysis to evaluate complement activation and related pathways. These biomarkers can include, but are not limited to, complement pathway proteins sC5b-9, C5a, C3a, total C3, Factor B and Ba, as well as cytokines associated with inflammation and disease; e.g., IL-1, IL-6, IL-8, IL-21, TNF-b, and MCP-1.

Individual serum concentration data for all participants who receive at least 1 dose of SOLIRIS and who have evaluable PK/PD data is used to summarize PK/PD parameters for SOLIRIS. Descriptive statistics are presented for all SOLIRIS® PK/PD endpoints at each sampling time. The PD effects of SOLIRIS® is summarized using absolute values and changes and percentage changes from baseline in serum pharmacodynamic markers over time (including but not limited to CH50, C5b9, other complement proteins), as appropriate. Exploratory serum and plasma biomarkers actual values and changes from baseline are summarized over time, as appropriate.

17. Immunogenicity

The presence of confirmed positive ADAs is summarized. Additionally, following confirmation of positive ADAs, samples are assessed for ADA titer and presence of neutralizing antibodies.

### Example 4: Data from COVID-19 Patients

SOLIRIS® was administered to subjects with COVID-19 and data on ten patients treated at various locations were analyzed. Two of the ten patients treated at Center #1 had their serum residual free eculizumab levels completely depleted by Day 4 after the initial 900 mg SOLIRIS® dose. They also had elevated C5b9 by that day, consistent with a return of complement activation. A third patient at Center #1 also showed come elevated complement levels, but the concern was not as marked or early as the other two. A patient in Center #2 similarly had elevated C5b9 by Day 6.

The data show that at least three out of eleven patients who had a return of terminal complement activity before the second dose on Day 8. This suggests that at least some of these patients with COVID-19 have massive activation of their complement. These data provide some further evidence that complement inhibition could provide a real therapeutic benefit for these patients, for example, via provision of extra drug early on in order to block the complement activity.

### Example 5: Eculizumab for Treatment of COVID-19

SOLIRIS® (eculizumab) is an effective and extensively studied terminal complement inhibitor, with a well-established safety profile. SOLIRIS has been investigated in numerous complement-mediated diseases and is currently approved in the European Union for 4 complement-mediated diseases (EU number: EU/1/07/393).

SOLIRIS® is proposed as an emergency therapy for the treatment of patients with confirmed SARS-CoV-2 infection with a clinical presentation consistent with COVID-19 severe pneumonia, acute lung injury, or acute respiratory distress syndrome (ARDS). It is hypothesized that maintaining complete terminal complement inhibition in these patients could ameliorate COVID-19-induced lung injury, improve outcomes in participants with COVID-19 pneumonia, and avoid the catastrophic consequences of immune-mediated lung injury or ARDS.

In recent weeks, multiple requests for access to SOLIRIS® for treatment of patients with COVID-19 severe infection have been received from physicians in France, Italy, and in the USA. As of 5 Apr. 2020, Investigators have been informed that 51 patients with COVID-19 who were critically ill have received SOLIRIS on a compassionate use basis in these countries (France: 15; Italy: 28, and USA: 8). Investigators have access to a limited quantity of data at this time.

The SOLIRIS® induction dosage regimen approved for the treatment of patients with aHUS, gMG, and NMOSD (900 mg once weekly for 4 doses) was proposed in the original ECU-COV-401 protocol. However, following examination of preliminary individual pharmacokinetic (PK) and pharmacodynamic (PD) data, some patients with COVID-19 receiving this dosage regimen demonstrated increased drug clearance and/or loss of PD control. Investigators amended the SOLIRIS dosing regimen in an effort to achieve immediate and complete terminal complement inhibition in patients with COVID-19 and as potential protection from the life-threatening consequences of complement-mediated injury.

1. Preliminary Eculizumab Pharmacokinetic/Pharmacodynamic Results in Patients with COVID-19

Preliminary PK and PD data for 7 patients with severe COVID-19 infection who were treated with SOLIRIS on a named-patient basis have been communicated to Investigators by treating physicians in France (through 5 Apr. 2020). Free eculizumab concentrations with time-matched soluble C5b9 levels in 6 patients, and CH50 (functional hemolysis assay) and time-matched soluble C5b9 in 1 patient

2. Results

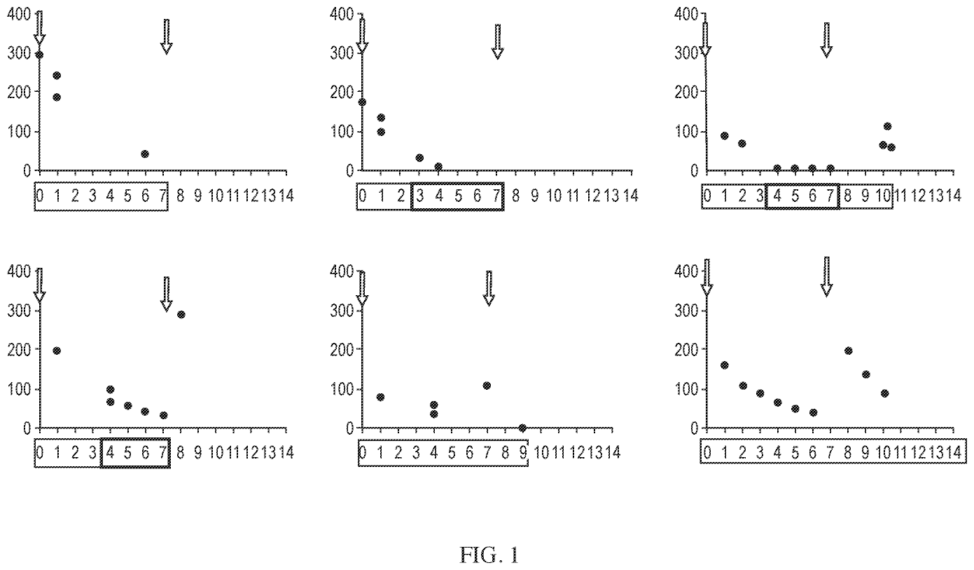

Individual plasma free eculizumab concentrations are shown in FIG. 1. The preliminary PK profiles following approved SOLIRIS® dosing in patients with COVID-19 show faster than expected eculizumab clearance in 3 of 6 patients, with corresponding sub-therapeutic eculizumab concentrations. The increased SOLIRIS® clearance is thought to be driven by an increased complement activation seen in some patients with COVID-19. Higher concentrations of circulating complement complexes are expected to bind more SOLIRIS®, thereby leading to a faster drug clearance relative to other indications.

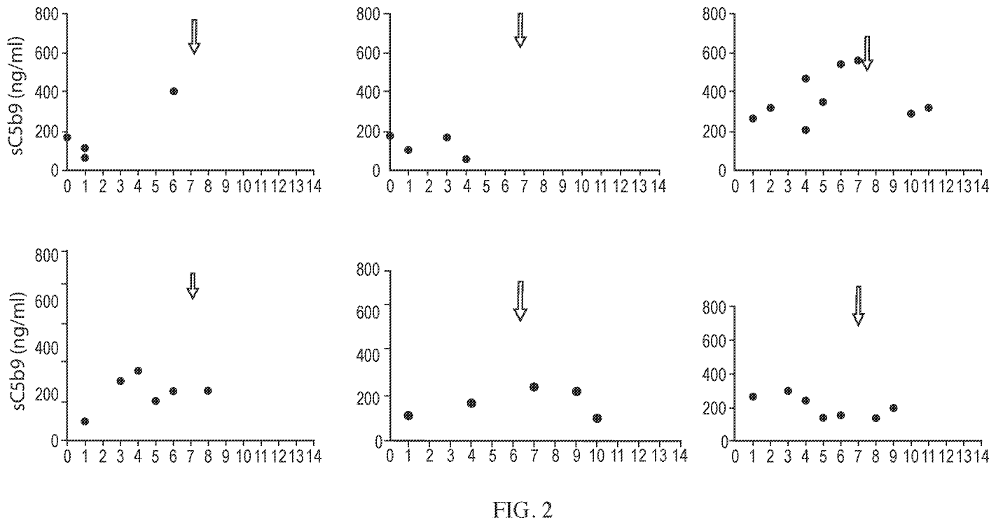

Individual plasma soluble C5b9 concentrations are shown in FIG. 2. Preliminary individual time-matched soluble C5b9 profiles support loss of complement inhibition in some patients when treated with the approved SOLIRIS® dosing.

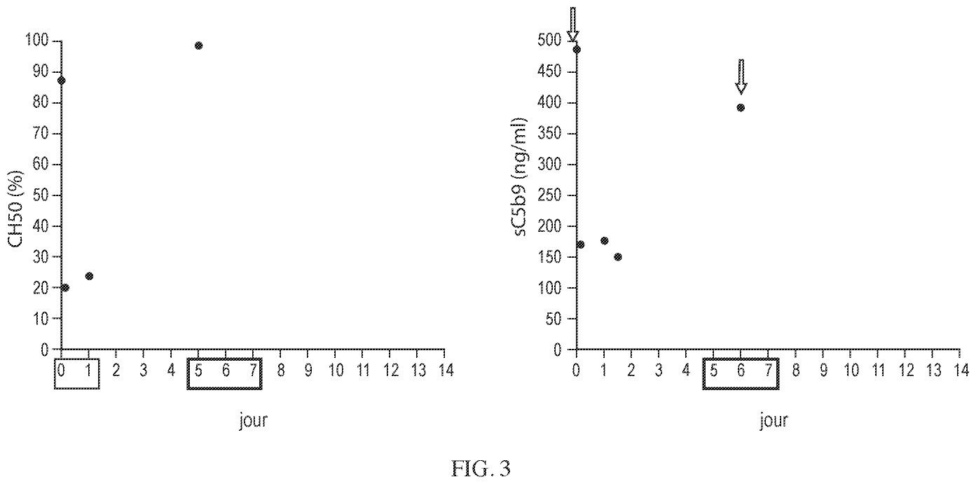

Individual CH50 and time-matched soluble C5b9 profiles are presented for one patient in FIG. 3. The individual results from two different PD assays (CH50 and sC5b9) suggest that the initial dose of 900 mg is insufficient to maintain terminal complement inhibition in this patient for the first week of treatment.

Examination of the preliminary individual PK/PD data suggests increased complement activation in the setting of COVID-19, with increased eculizumab clearance and corresponding subtherapeutic eculizumab concentration. Individual concentrations of eculizumab and serum C5b9 along with results from CH50 functional hemolysis assay in patients with COVID-19 show that the approved SOLIRIS® induction dosing for patients with aHUS, gMG, and NMOSD (900 mg once weekly for 4 doses) is insufficient to maintain complete terminal complement inhibition in all patients.

3. Conclusions

The approved dosage regimens of SOLIRIS® were designed to achieve immediate, complete, and sustained inhibition of terminal complement across the different indications approved. The present investigators' cumulative experience with SOLIRIS® in patients with PNH, aHUS, gMG, and NMOSD supports complete terminal complement inhibition as the correlate of efficacy. Data presented above demonstrate that at least 3 patients did not achieve complete terminal complement inhibition throughout the dosing interval. One of the 3 patients died. The apparent need for complete terminal complement inhibition is the basis of the therapeutic strategy supporting treatment with SOLIRIS® in participants with COVID-19 who present with severe pneumonia, acute lung injury, or ARDS.

Based on these preliminary findings and the importance of complete terminal complement inhibition to ameliorate COVID-19-induced lung injury, higher and more frequent eculizumab dosing is expected to address the increased drug clearance resulting from complement amplification, particularly in the first 2 weeks of treatment when complement would be expected to be most amplified. Complement amplification is noted in other clinical settings (Jodele et al. (Biol Blood Marrow Transplant. 2016; 22(2):307-315); Peffault de Latour et al. (Blood. 2015; 125(5):775-783)), also necessitating more frequent early SOLIRIS dosing to counter the increased eculizumab clearance and maintain terminal complement inhibition.

Similar dosing levels of SOLIRIS® to those proposed in this document have been previously studied in Studies C10-001 and C10-002, global studies exploring safety and efficacy in transplant populations that included conduct in a number of sites in France. Both studies used the following dosing regimen: eculizumab 1200 mg prior to allograft transplantation (Day 0, starting approximately 1 hour prior to kidney allograft reperfusion), eculizumab 900 mg (Days 1, 7, 14, 21, and 28), and eculizumab 1200 mg (Weeks 5, 7, and 9). The dosing regimen, with more frequent early dosing to address complement amplification in a post-transplant setting, was well tolerated and consistent with the expected safety profile of SOLIRIS with no new safety concerns (Marks et al. (Am J Transplant. 2019; 19(10):2876-2888)).

To account for the observed SARS-CoV-2-related amplification of complement, the proposed SOLIRIS® dosing regimen will be administered intravenously at a dose of 1200 mg on Days 1, 4, and 8 and 900 mg on Days 15 and 22. Optional doses of 900 or 1200 mg can be administered on Days 12 and 18, per the discretion of the Investigator in consultation with the Medical Monitor.

The proposed eculizumab dose regimen amendment to treat patients with COVID-19 is based on an empiric assessment of preliminary PK/PD results and utilizes an understanding of SOLIRIS® PK and PD dosing goals for achieving immediate, complete, and sustained terminal complement inhibition.

The potential risks identified in association with the administration of SOLIRIS® are justified by the anticipated benefits in an effort to achieve immediate and complete terminal complement inhibition that may be afforded to participants with COVID-19 severe pneumonia, acute lung injury, or ARDS.

### Example 6: Safety and Efficacy Study of IV ULTOMIRIS® (Ravulizumab) in Patients with COVID-19 Severe Pneumonia

A Phase 3 open-label, randomized, controlled study (also referred to as “ALXN1210-COV-305”; version 1) is conducted to evaluate the safety and efficacy of intravenously administered ravulizumab compared with best supportive care in patients with COVID-19 Severe Pneumonia, Acute Lung Injury, or Acute Respiratory Distress Syndrome.

1. Objectives and Study Endpoints

The primary objective is to evaluate the effect of ravulizumab plus best standard care (BSC) compared with BSC alone on the survival of patients with COVID 19 (e.g., as assessed by survival (based on all-cause mortality) at Day 29). The secondary objective is to evaluate the efficacy of ravulizumab and best supportive care compared with best supportive care alone on outcomes in patients with COVID 19 (e.g., as assessed by number of days free of mechanical ventilation at Day 29, change from baseline in SpO2/FiO2 at Day 29, duration of intensive care unit stay at Day 29, change from baseline in Sequential Organ Failure Assessment (SOFA) score at Day 29, and duration of hospitalization at Day 29).

The safety objective is to characterize the overall safety of ravulizumab plus BSC compared with BSC alone in patients with COVID 19 (e.g., as assessed by TEAEs and TESAEs).

Additional objectives include characterizing the pharmacokinetic (PK)/pharmacodynamic (PD), and immunogenicity of ravulizumab in patients with COVID 19 (e.g., as assessed by change in serum ravulizumab concentrations over time, change in serum free C5 concentrations over time, and incidence and titer of anti-ALXN1210 antibodies and neutralizing antibodies).

The objective with respect to biomarkers is to assess the effect of C5 inhibition on systemic activation of complement and inflammation in patients with COVID 19 (e.g., as assessed by the change in absolute levels of soluble biomarkers in blood and urine associated with complement activation and inflammatory processes over time).

The exploratory objective is to evaluate (1) the effect of ravulizumab plus BSC compared with BSC alone on the 60 and 90 day survival of patients with COVID 19 (e.g., as assessed by survival (based on all-cause mortality) at Day 60 and Day 90) and (2) the effect of ravulizumab plus BSC compared with BSC alone on progression to renal failure requiring dialysis in patients with COVID 19 (e.g., as assessed by incidence of progression to renal failure requiring dialysis at Day 29).

Baseline represents assessments/procedures that are performed on or before the first infusion of study drug is administered on Day 1 (for patients randomized to ravulizumab plus BSC) and on or before initiation of assessments/procedures on Day 1 (for patients randomized to BSC).

2. Study Design

Study ALXN1210-COV-305 is a multicenter Phase 3, open label, randomized, controlled study designed to evaluate the safety and efficacy of intravenous (IV) ravulizumab compared with best supportive care (BSC) in patients with a confirmed diagnosis of SARS CoV 2 infection, and a clinical presentation consistent with COVID 19 severe pneumonia, acute lung injury, or ARDS. Patients at least 18 years of age, weighing≥40 kg, and admitted to a designated hospital facility for treatment are screened for eligibility in this study. Accounting for a 10% nonevaluable rate, approximately 270 patients are randomized in a 2:1 ratio (180 patients to receive ravulizumab+BSC, 90 patients to BSC alone).

Patients randomized to ravulizumab plus BSC receive a weight based dose of ravulizumab on Day 1 as set forth in Table 9. For patients still requiring mechanical ventilation or who exhibit evidence of ongoing end-organ damage in the judgment of the Investigator, 900 mg of ravulizumab can be administered on Day 15. Dose to be administered as follows: Patients≥40 to <60 kg: 2400 mg/kg; ≥60 to <100 kg: 2700 mg/kg; and ≥100 kg: 3000 mg/kg on Day 1. All patients continue to receive medications, therapies, and interventions per standard hospital treatment protocols for the duration of the study.

The study consists of a Screening Period of up to 3 days, a Primary Evaluation Period of 4 weeks, a final assessment at Day 29 or upon discharge, and 2 Follow up Visits at Day 60 and Day 90. The Follow up Visits are conducted as a telephone call if the patient is discharged from the hospital or an in person visit if the patient is still hospitalized. The total duration of each patient's participation is anticipated to be approximately 4 months (Scheme 1).

Screening and the Day 1 visits can occur on the same day if the patient has met all inclusion and no exclusion criteria.

3. Description of Study Drug

Ravulizumab, a recombinant humanized anti-C5 mAb composed of two 448 amino acid heavy chains and two 214 amino acid light chains, is an IgG2/4 kappa immunoglobulin consisting of human constant regions, and murine complementarity-determining regions grafted onto human framework light- and heavy-chain variable regions. Ravulizumab is produced in Chinese hamster ovarian cell lines and was designed through minimal targeted engineering of eculizumab by introducing 4 unique amino acid substitutions to its heavy chain to extend antibody half-life.

Ravulizumab drug product is supplied for clinical studies as a sterile, preservative-free 10 mg/mL solution in single-use vials and designed for infusion by diluting into commercially available saline (0.9% sodium chloride injection; country-specific pharmacopeia) for administration via IV infusion.

The proposed dosage regimen for the treatment of patients with COVID-19 who are ≥18 years and ≥40 kg and are randomized to ravulizumab+BSC is presented in Table 9.

TABLE 9
Ravulizumab Dosage Regimen for COVID-19 Severe Pneumonia,
Acute Lung Injury, or Acute Respiratory Distress Syndrome

| Patient Body | | Optional Dose |
|---|---|---|
| Weight (kg) | Dose on Day 1 (mg) | on Day 15 (mg)¹ |
| ≥40 to <60 | 2400 | 900 |
| ≥60 to <100 | 2700 | 900 |
| ≥100 | 3000 | 900 |

¹A Day 15 dose of 900 mg ravulizumab is administered to patients still requiring mechanical ventilation or who exhibit evidence of ongoing end-organ damage in the judgment of the Investigator.

Ravulizumab drug product is formulated at pH 7.0 and each 30 mL vial contains 300 mg of ravulizumab, 0.02% polysorbate 80, 150 mM sodium chloride, 6.63 mM sodium phosphate dibasic, 3.34 mM sodium phosphate monobasic, and Water for Injection, United States Pharmacopeia.

The ravulizumab admixture is administered to the patient using an IV tubing set via an infusion pump followed by an IV flush. Use of a 0.2 micron filter is required during the infusion. The IV flush is infused at the same rate of the infusion and end of flush is considered the end-of-infusion. The IV flush volume is not to be included in the total volume of study drug administered.

Stability studies of the diluted admixture of ravulizumab (10 mg/mL) in 0.9% sodium chloride injection support an in-use stability of 6 hours at room temperature at 23° C.-27° C. (73° F.-80° F.) and 24 hours when refrigerated at 2° C.-8° C. (36° F.-46° F.). Ravulizumab vials are not to be frozen or shaken.

4. Duration of Treatment and End of Study Definition

For each patient, the total duration of the study is anticipated to be up to approximately 3 months and consists of the following:
1. Approximately 4 weeks while the patient is hospitalized: Up to 3 days for Screening, 4 weeks for the Primary Evaluation Period, and a final assessment at Day 29 or on day of discharge, whichever occurs first.
2. 2 follow-up visits conducted approximately 4 weeks apart on Day 60 and Day 90 (which may be conducted as a telephone call if the patient is discharged from the hospital or an in-person visit if the patient is still hospitalized).

The end of the Primary Evaluation Period is defined as the date when the last surviving patient completes the Day 29/early termination (ET) visit. The end of the study is defined as the last patient's last visit, which may be the final safety follow-up telephone call or in-person visit.

5. Study Population

Patients are included in the study if they meet the following criteria:
1. Male or female patients at least 18 years of age and at least 40 kg at the time of providing informed consent;
2. Confirmed diagnosis of SARS-CoV-2 infection presenting as severe COVID-19 requiring hospitalization;
3. Severe pneumonia, acute lung injury, or ARDS confirmed by computed tomography (CT) or X-ray at Screening or within the 3 days prior to Screening;
4. Severe pneumonia, acute lung injury, or ARDS requiring oxygen supplementation with invasive or noninvasive mechanical ventilation (WHO, 2020);
5. Female patients of childbearing potential and male patients with female partners of childbearing potential must follow protocol-specified guidance for avoiding pregnancy while on treatment and for 8 months after treatment with the single dose of study drug; and
6. Informed consent must be provided for all patients. If allowable per local regulations, exceptions may be granted in cases where the patient is unable to provide informed consent.

Patients are excluded from the study if they meet any of the following criteria:
1. Patient is not expected to survive more than 24 hours;
7. Patient is on invasive ventilation for more than 48 hours prior to Screening;
8. Use of the following medications and therapies: (a) current treatment with a complement inhibitor, (b) Rituximab within 3 months of Screening, (c) Mitoxantrone within 3 months of Screening, or (d) intravenous immunoglobulin (IVIg) within 3 weeks prior to Screening;
9. Patient has an unresolved Neisseria meningitidis infection;
10. History of hypersensitivity to any ingredient contained in the study drug, including hypersensitivity to murine proteins;
11. Female patients who have a positive pregnancy test result at Screening or on Day 1;
12. Severe pre-existing cardiac disease (i.e., New York Heart Association Class 3 or Class 4, acute coronary syndrome or persistent ventricular tachyarrhythmias); or
13. Participation in another interventional treatment study within 30 days before initiation of ravulizumab on Day 1 in this study or within 5 half-lives of that investigational product, whichever is greater.

6. Statistical Considerations

A sample size of 243 patients (162 ravulizumab plus BSC; 81 BSC alone) is required to ensure at least 90% power and detect an improvement in survival from 60% on the BSC group to 80% on the ravulizumab+BSC group at Day 29.

This sample size calculation assumes: (a) 1-sided Z-test of the difference in 2 proportions, (b) Type I error=0.025, (c) Pooled variance, (d) 2:1 randomization on the 2 treatment groups, and (e) one interim analysis at 50% information which is after collecting primary efficacy data on approximately 122 patients. The early stopping boundaries for Efficacy and Futility are constructed using α-spending function as Lan-DeMets spending function with O'Brien-Fleming flavor and β-spending function as Gamma(-4). Considering a nonevaluable rate of 10%, this study is planned to randomize approximately 270 patients (180 ravulizumab plus BSC; 90 BSC alone).

This is an open-label study. Eligible patients who meet all inclusion and no exclusion criteria will be randomized in a 2:1 ratio to receive either ravulizumab plus BSC or BSC alone. Randomization is stratified by invasive or noninvasive mechanical ventilation on Day 1. A randomization schedule is developed by a centralized third-party.

The Full Analysis Set (FAS) consists of all randomized patients who receive at least 1 dose of ravulizumab for patients randomized to ravulizumab plus BSC or who were randomized to BSC alone. The FAS is used for the analysis of efficacy data and is considered the primary analysis population.

The Per-Protocol Set (PPS) is a subset of the FAS without any important protocol deviations that could impact efficacy analyses. Determination of applicable important protocol deviations for this purpose are made prior to database lock. The PPS is used for sensitivity analyses of the primary and secondary efficacy endpoints.

The Safety Set is identical to the FAS and consists of all randomized patients who receive at least 1 dose of ravulizumab for patients randomized to ravulizumab plus BSC or who were randomized to BSC alone. The Safety Set is used for the analysis of safety data.

The primary analysis is conducted when all patients have completed the Primary Evaluation Period. This analysis includes all efficacy, safety, and PK/PD/immunogenicity study data for regulatory submission purposes and is the final analysis of the Primary Evaluation Period.

Summary statistics are presented by treatment group and by visit, where applicable. Descriptive statistics for continuous variables minimally include the number of patients, mean, standard deviation, median, minimum, and maximum. For categorical variables, frequencies and percentages will be presented. Graphical displays are provided as appropriate. All statistical analyses re performed based on a 2-sided Type I error of 5%, unless otherwise noted. Baseline represents assessments/procedures that are performed on or before the first and only infusion of study drug is administered on Day 1 (for patients randomized to

ravulizumab plus BSC) and before initiation of assessments/procedures on Day 1 (for patients randomized to BSC alone).

Analyses are performed using SAS® software Version 9.4 or higher.

The primary efficacy endpoint is survival (based on all-cause mortality) at Day 29 and is compared between the 2 treatment groups using a 1-sided Z-test of the difference in 2 proportions with a pooled variance and a type I error of 0.025. The estimated risk difference is summarized along with the 95% confidence interval. If a patient is discharged before Day 29, he/she is considered as survived at Day 29.

Survival is also analyzed using the method of Kaplan and Meier (KM) and compared using a log-rank test as a sensitivity analysis. Hazard ratio and risk reduction is summarized from a Cox proportional hazards model. Confidence intervals (95%) are presented for the survival estimate at Day 29 based on the complementary log-log transformation. Kaplan-Meier curves for both treatment groups are produced.

A sensitivity analysis of the primary endpoint is also performed using a 3-level categorical outcome of 3) alive and discharged from ICU; 2) alive, in ICU, and off mechanical ventilation; or 1) death. The 2 treatment groups is compared using a chi-squared test. Additional sensitivity analyses includes statistical models adjusting for age, randomization stratification factor, and other important covariates. The Statistical Analysis Plan (SAP) describes the sensitivity analyses in greater detail.

An interim analysis of the primary endpoint is also conducted. Number of days free of mechanical ventilation at Day 29 is compared between treatment groups using an analysis of covariance (ANCOVA), adjusting for age and randomization stratification factor, among survivors. If a patient is discharged from the hospital prior to Day 29, he/she is considered alive and free of mechanical ventilation for the remaining days up to Day 29.

Change from baseline in SpO2/FiO2 at Day 29 is analyzed using a mixed model for repeated measures (MMRM) with baseline SpO2/FiO2, age, randomization stratification factor, treatment group indicator, study day, and study day by treatment group interaction as fixed covariates. All patients who survive to Day 29 are included in the model, except those patients without any postbaseline scores. Sensitivity analyses include imputations for missing data. Change from baseline in PaO2/FiO2 at Day 29 is also analyzed using a MMRM with baseline PaO2/FiO2, age, randomization stratification factor, treatment group indicator, study day, and study day by treatment group interaction as fixed covariates. All patients with PaO2/FiO2 data who survive to Day 29 are included in the model, except those without any postbaseline scores. Sensitivity analyses include imputations for missing data. Changes from baseline in SpO2/FiO2 and PaO2/FiO2 are also summarized for nonsurvivors.

Duration of intensive care unit (ICU) stay at Day 29 is compared between treatment groups using an ANCOVA, adjusting for age and randomization stratification factor, among survivors. Duration of ICU stay at Day 29 is also be summarized for nonsurvivors.

Change from baseline in SOFA score at Day 29 is analyzed in a similar manner as change from baseline in SpO2/FiO2, using an MMRM and including baseline SOFA score. Duration of hospitalization at Day 29 is analyzed in a similar manner as duration of ICU stay.

A closed testing procedure is applied to control the type I error for the analyses of the primary and secondary endpoints. If the primary endpoint is statistically significant in favor of ravulizumab, the secondary endpoints is evaluated according to the following rank order:
1. Number of days free of mechanical ventilation at Day 29
2. Change from baseline in SpO2/FiO2 at Day 29
3. Duration of ICU stay at Day 29
4. Change from baseline in SOFA score at Day 29
5. Duration of hospitalization at Day 29

The hypothesis testing proceeds from highest rank (#1) the number of days free of mechanical ventilation at Day 29 to the lowest rank (#5) duration of hospitalization at Day 29, and if statistical significance is not achieved at an endpoint (p≥0.05), then endpoints of lower rank is not considered to be statistically significant. Confidence intervals and p-values are presented for all secondary efficacy endpoints for descriptive purposes, regardless of the outcome of the closed testing procedure.

All safety analyses are made on the Safety Population. Safety results are reported by treatment group.

The analysis and reporting of AEs and SAEs are based on treatment-emergent AEs (TEAEs) and SAEs (TESAEs), defined as AEs and SAEs with onset during or after treatment with the single dose of ravulizumab. The incidence of TEAEs and TESAEs is summarized by System Organ Class (SOC) and Preferred Term, with additional summaries showing relationship to ravulizumab, severity, TEAEs or TESAEs leading to ravulizumab discontinuation, and TESAEs resulting in death.

Laboratory measurements, as well as their changes from baseline, at each visit and shift from baseline, if applicable, are summarized. Vital sign measurements and physical examination findings are also summarized over time.

Blood samples are to be collected for pharmacokinetic (PK) and free C5 analysis. Individual serum concentration data for all patients who receive at least 1 dose of ravulizumab and who have evaluable PK/pharmacodynamic (PD) data are used to summarize PK/PD parameters for ravulizumab. Descriptive statistics are presented for all ravulizumab PK/PD endpoints at each sampling time. The PD effects of ravulizumab is summarized using absolute values and changes and percentage changes from baseline in free C5 serum concentrations over time, as appropriate.

Serum samples are to be collected at screening and following treatment, according to the Schedule of Activities for biomarker analysis to evaluate complement activation and related pathways. These biomarkers may include, but are not limited to complement pathway proteins sC5b-9, C5a, C3a, total C3, Factor B and Ba, as well as cytokines associated with inflammation and disease, e.g., interleukin (IL)-1, IL-6, IL-8, IL-21, tumor necrosis factor (TNF)-b, and monocyte chemoattractant protein (MCP)-1; and markers associated with cardiovascular disease, procalcitonin, myoglobin, high sensitivity troponin I and N-terminal pro b-type natriuretic peptide.

Serum, urine, and plasma biomarkers' actual values and changes from baseline are summarized over time, as appropriate.

The incidence and titers for antidrug antibodies (ADAs) to ALXN1210, are summarized in tabular format by treatment group. The proportion of patients ever positive and the proportion of patients always negative may be explored. Confirmed ADA positive samples is evaluated for the presence of neutralizing antibodies.

Survival (based on all-cause mortality) at Day 60 and Day 90 is estimated using the Kaplan-Meier method and compared using a log-rank test. Hazard ratio and risk reduction are summarized from a Cox proportional hazards model. Confidence intervals (95%) are presented for the survival estimates at Days 60 and 90 based on the complementary log-log transformation. Kaplan-Meier curves for both treatment groups are produced.

Incidence of progression to renal failure requiring dialysis at Day 29 is analyzed in a similar manner as the primary endpoint.

An interim analysis for efficacy and futility conducted when approximately 122 patients have completed Day 29 (or were early terminated [ET]). If the stopping criteria are met, the study may be terminated early for efficacy or futility depending on which stopping boundary is crossed. The early stopping boundaries for efficacy and futility are constructed using α-spending function as Lan-DeMets (O'Brien-Fleming) spending function and β-spending function as Gamma(-4). A 1-sided Z-test of the difference in 2 proportions will used with a pooled variance and a type I error of 0.025.

The final primary analysis is conducted when all patients have completed the Primary Evaluation Period. This analysis includes all efficacy, safety, and PK/PD/immunogenicity study data for regulatory submission purposes. This analysis is not considered an interim analysis.

7. Schedule of Activities

A schedule of activities is presented in Table 10.

TABLE 10
Schedule of Activities

| | Period | | | | | | | |
|---|---|---|---|---|---|---|---|---|
| | Screening | | | | | | Follow- | |
| | Period | Primary Evaluation Period | | | | | up³ | |
| | Study Day (D) | | | | | | | |
| | D −3 | | | | | D29 | | |
| | to D −1 | D1^{1,2} | D8 | D15² | D22 | or ET | D60 | D90 |
| | Study Window (days) | | | | | | | |
| | NA | NA | ±1 | ±1 | ±1 | ±1 | +7 | |
| Eligibility | | | | | | | | |
| Informed consent | X | | | | | | | |
| Inclusion/exclusion | X | | | | | | | |
| Medical history | X | | | | | | | |
| Demographics | X | | | | | | | |
| Study Administrative | | | | | | | | |
| Confirmation of vaccination or | X | | | | | | | |
| appropriate antibiotic prophylaxis | | | | | | | | |
| against N. meningitidis⁴ | | | | | | | | |
| Screening Laboratory Tests | | | | | | | | |
| Confirmed positive SARS-COV-2 test | X | | | | | | | |
| Chest CT or X-ray⁵ | X | | | | | | | |

| | | | | |
|---|---|---|---|---|
| Serum follicle stimulating hormone | X | | | |
| Pregnancy test⁶ | X | | | X |
| Randomization | | | | |
| Randomized to study drug + BSC | | X | | |
| or BSC alone | | | | |
| Administration of Study Drug | | | | |
| Ravulizumab | | X | X² | |
| Efficacy Assessments | | | | |

| | | |
|---|---|---|
| Survival status | <<Monitor continuously>> | X |
| Mechanical ventilation status | <<Monitor continuously>> | |
| Hospitalization status | <<Monitor continuously>> | |

| Intensive care unit status | <<Monitor continuously>> |
|---|---|
| Dialysis status | <<Monitor continuously>> |

| FiO2, SpO2, PaO2⁷ | | <<Monitor continuously>> | | | | | |
|---|---|---|---|---|---|---|---|
| Sequential Organ Failure | X | X | X | X | X | X | |
| Assessment score | | | | | | | |
| Safety Assessments | | | | | | | |
| Electrocardiogram | X | | | | | | |
| Physical examination⁸ | X | X | X | X | X | X | |
| Glasgow Coma Scale | X | X | X | X | X | X | |
| Body weight | X | X | X | X | X | X | |
| Vital sign measurements⁹ | X | X | X | X | X | X | |
| Adverse event review and | <<Monitor continuously>> | | | | | | X |
| evaluation | | | | | | | |
| Review safety card¹⁰ | | X¹⁰ | | | | X¹⁰ | X¹⁰ |
| Safety Laboratory Tests¹¹ | | | | | | | |
| Clinical chemistry | X | X | X | X | X | X | |
| Hematology | X | X | X | X | X | X | |
| Coagulation panel and D-dimer | X | X | X | X | X | X | |
| Urinalysis | X | X | X | X | X | X | |
| Direct Coombs Test | X | | | | | | |
| PK/PD/Immunogenicity Tests | | | | | | | |
| PK and free C5¹² | | X | X | X | X | X | |
| Immunogenicity | | X | | | | X | |
| Biomarker Tests | | | | | | | |

| | | | | | |
|---|---|---|---|---|---|
| Serum, urine, and plasma | X | X | X | X | X |
| biomarkers¹³ | | | | | |
| Other | | | | | |

| | | |
|---|---|---|
| Concomitant medication¹⁴ | X | <<Monitor continuously>> |

¹The Day 1 visit can occur on the same day as Screening.
²Patients randomized to ravulizumab plus BSC receive a weight-based dose of ravulizumab on Day 1. A Day 15 dose of 900 mg ravulizumab is administered to patients still requiring mechanical ventilation or who exhibit evidence of ongoing end-organ damage in the judgment of the Investigator.
³Safety follow-up monitoring is conducted once a month for 3 months to review patient status, including survival and pregnancy, and to obtain information about new or worsening TESAEs. The follow up is conducted as a telephone call if the patient is discharged from the hospital or an in-person visit if the patient is still hospitalized.
⁴Confirmation of meningococcal vaccination within the past 5 years prior to dosing for patients randomized to ravulizumab. If vaccination cannot be confirmed, the patient receives prophylactic antibiotics prior to initiating ravulizumab treatment and for at least 8 months from the last infusion of ravulizumab. If patients are vaccinated after treatment with ravulizumab, they continue antibiotic prophylaxis for at least 2 weeks after meningococcal vaccination.
⁵Can be performed at Screening or within the 3 days prior to Screening.
⁶Urine or serum pregnancy tests (beta human chorionic gonadotropin) to be performed in female patients of childbearing potential only. A negative pregnancy test result is required before administration of the first dose of ravulizumab.
⁷SpO2 is measured by pulse oximetry. PaO2 is measured by arterial blood gas, if available. FiO2 to be measured by supplemental oxygen. The highest daily measurement on the lowest inspired supplemental oxygen level are recorded in the CRF/eCRF.
⁸Full or abbreviated physical examination. A full physical examination includes, at a minimum, assessments of the following organs/body systems: skin, head, ears, eyes, nose, throat, neck, lymph nodes, chest, heart, abdomen, extremities, and musculoskeletal. An abbreviated physical examination consists of at least an evaluation of the respiratory system.
⁹Vital sign measurements should include systolic and diastolic BP (millimeters of mercury [mm Hg]), heart rate (beats/minute), respiratory rate (breaths/minute), and temperature (degrees Celsius [° C.] or degrees Fahrenheit [° F.]). These measurements are taken predose on Day 1.
¹⁰Review the Patient Safety Information Card with the patient (including discussion of the risks associated with study drug treatment, such as meningococcal infection) at the time of dosing and discharge. Upon discharge, the patients who receive ravulizumab must carry the Patient Safety Information Card at all times and for at least 8 months after the single infusion of study drug (if randomized to BSC plus ravulizumab).
¹¹Clinical safety laboratory measurements are collected predose on Day 1.
¹²Serum samples for PK and free C5 analyses are collected at the timepoints indicated in the Schedule of Activities. On Day 1, PK/PD samples are collected within 90 minutes before the administration of ravulizumab (predose) and within 60 minutes after the end-of-infusion (postdose). Postdose samples are collected from a separate line or needle stick to the noninfused arm, not from the infusion line. Samples are collected at any time after Day 1 during the Primary Evaluation Period.
¹³Serum samples for biomarker analyses are collected at the indicated visits and stored at the investigational site prior to analysis. Samples are collected predose (any time before infusion start).
¹⁴Concomitant medications considered relevant to the treatment of COVID-19 or ravulizumab treatment (e.g., antimicrobials, antimalarials, antivirals, steroids, and vasopressors) that the patient is receiving at the time of Screening must be recorded on the eCRF.

8. Vaccination and Prophylactic Antibiotics

It is anticipated that patients who have not received a meningococcal vaccination within the past 5 years may be unable to receive meningococcal vaccinations prior to initiating treatment with ravulizumab during this study. If vaccination cannot be confirmed, the patient receives prophylactic antibiotics against meningococcal infection prior to initiating ravulizumab treatment and for at least 8 months from the last infusion of ravulizumab.

When patients can be vaccinated, vaccines against meningococcal serotypes A, C, Y, W135, and B, where available, are recommended to prevent common pathogenic meningococcal serotypes. Patients must be vaccinated or revaccinated according to current national vaccination guidelines or local practice for vaccination use with complement inhibitors (e.g., ravulizumab). Vaccination may not be sufficient to prevent meningococcal infection. Consideration should be given per official guidance and local practice on the appropriate use of antibacterial agents. If patients are vaccinated after treatment with ravulizumab, they should continue antibiotic prophylaxis for at least 2 weeks after meningococcal vaccination.

9. Protocol-Required Laboratory Tests

Protocol-required laboratory tests are set forth in Table 11.

TABLE 11
Protocol-Required Laboratory Tests

| | |
|---|---|
| Hematology Panel | Clinical Chemistry Panel |
| Haptoglobin | Alanine aminotransferase |
| Hematocrit | Aspartate aminotransferase |
| Hemoglobin | Blood urea nitrogen |
| Platelet count | Complement-reactive protein |
| White Blood Cell (WBC) count | Creatinine |
| WBC differential | Lactate dehydrogenase |
| Coagulation Panel | Sodium |
| D-dimer | Total bilirubin (direct and indirect) |
| International normalized ratio | Ferritin |
| Partial thromboplastin time | Other |
| Prothrombin time | Antidrug antibody |
| Urinalysis | Arterial blood gas |
| Albumin | Beta human chorionic gonadotropin (females |
| Creatinine | of childbearing potential only) |
| Protein | Direct Coombs test |
| Inflammatory Biomarkers | Free C5 |
| Interleukin-1 (IL-1), IL-6, IL-8, and IL-21 | Pharmacokinetic assay |
| Complement Pathway Biomarkers | Serum follicle-stimulating hormone |
| sC5b-9, C5a, C3a, total C3, Factor B, and | (postmenopausal females only) |
| Factor Ba | Other Biomarkers |
| Cardiac Biomarkers | monocyte chemoattractant protein-1 (MCP-1) |
| Procalcitonin | tumor necrosis factor-b (TNF-b) |
| Myoglobin | |
| N-terminal pro b-type natriuretic peptide | |
| (NT-proBNP) | |
| high sensitivity troponin I (hs-TnI) | |

### Example 7

The protocol of Example 6 is incorporated by reference; wherein dosing is carried out as follows:

ULTOMIRIS® (intravenous) dosing:

Day 1: Labelled weight-based loading dose per United States Product Information (USPI) label for ULTOMIRIS® (ravulizumab-cwvz) injection, for intravenous use, e.g., for PNH.

Day 5: 900 mg (or 600 mg for patients<60 kg)

Day 10: 900 mg (or 600 mg for patients<60 kg)

Day 15: 900 mg for all patients

It is believed that this regimen addresses the high complement activation observed in patients with COVID-19, ensuring that the patients are adequately covered, and providing the maximal chance to see efficacy in the clinical studies.

Exemplary loading doses are shown in Tables 12 and 13, below:

TABLE 12
ULTOMIRIS ® (ravulizumab) Weight-
Based Dosing Regimen - PNH

| Body Weight Range (kg) | Loading Dose (mg) |
|---|---|
| greater than or equal to 40 to less than 60 | 2,400 |
| greater than or equal to 60 to less than 100 | 2,700 |
| greater than or equal to 100 | 3,000 |

TABLE 13
ULTOMIRIS ® (ravulizumab) Weight-
Based Dosing Regimen - aHUS

| Body Weight Range (kg) | Loading Dose (mg) |
|---|---|
| greater than or equal to 5 to less than 10 | 600 |
| greater than or equal to 10 to less than 20 | 600 |
| greater than or equal to 20 to less than 30 | 900 |
| greater than or equal to 30 to less than 40 | 1,200 |
| greater than or equal to 40 to less than 60 | 2,400 |
| greater than or equal to 60 to less than 100 | 2,700 |
| greater than or equal to 100 | 3,000 |

### Example 8: Efficacy and Safety Study of IV Ravulizumab in Patients with COVID 19 Severe Pneumonia

A Phase 3 Open-label, Randomized, Controlled Study to Evaluate the Efficacy and Safety of Intravenously Administered Ravulizumab Compared with Best Supportive Care in Patients with COVID-19 Severe Pneumonia, Acute Lung Injury, or Acute Respiratory Distress Syndrome (NCT04369469; first posted; 30 Apr. 2020; Smith et al., Trials. 2020; 21: 639).

The primary objective of the trial is to evaluate the effect of ravulizumab plus BSC compared with BSC alone on the survival of patients with COVID 19 (e.g., as assessed by survival (based on all cause mortality) at Day 29).

The secondary objective is to evaluate the efficacy of ravulizumab plus BSC compared with BSC alone on outcomes in patients with COVID 19 (e.g., as assessed by (1) number of days free of mechanical ventilation at Day 29, (2) change from baseline in SpO2/FiO2 at Day 29, (3) duration of intensive care unit stay at Day 29, (4) change from baseline in SOFA score at Day 29, and (5) duration of hospitalization at Day 29.

The safety objective is to characterize the overall safety of ravulizumab plus BSC compared with BSC alone in patients with COVID 19 (e.g., as assessed by incidence of TEAEs and TESAEs).

With respect to pharmacokinetic/pharmacodynamic/immunogenicity, the objective is to characterize the PK/PD and immunogenicity of ravulizumab in patients with COVID 19 (e.g., as assessed by (1) change in serum ravulizumab concentrations over time, (2) change in serum free C5 concentrations over time, and (3) incidence and titer of anti ALXN1210 antibodies).

With respect to biomarkers, the objective is to assess the effect of C5 inhibition on systemic activation of complement and inflammation in patients with COVID 19 (e.g., as assessed by change in absolute levels of soluble biomarkers in blood and urine associated with complement activation and inflammatory processes over time).

Exploratory objectives include: (1) evaluating the effect of ravulizumab plus BSC compared with BSC alone on the 60 and 90 day survival of patients with COVID 19 (e.g., as assessed by survival (based on all-cause mortality) at Day 60 and Day 90) and (2) evaluating the effect of ravulizumab plus BSC compared with BSC alone on progression to renal failure requiring dialysis in patients with COVID 19 (e.g., as assessed by incidence of progression to renal failure requiring dialysis at Day 29).

Baseline represents assessments/procedures that are performed on or before the infusion of ravulizumab on Day 1 (for patients randomized to ravulizumab plus BSC) and on or before initiation of assessments/procedures on Day 1 (for patients randomized to BSC alone).

1. Overall Design

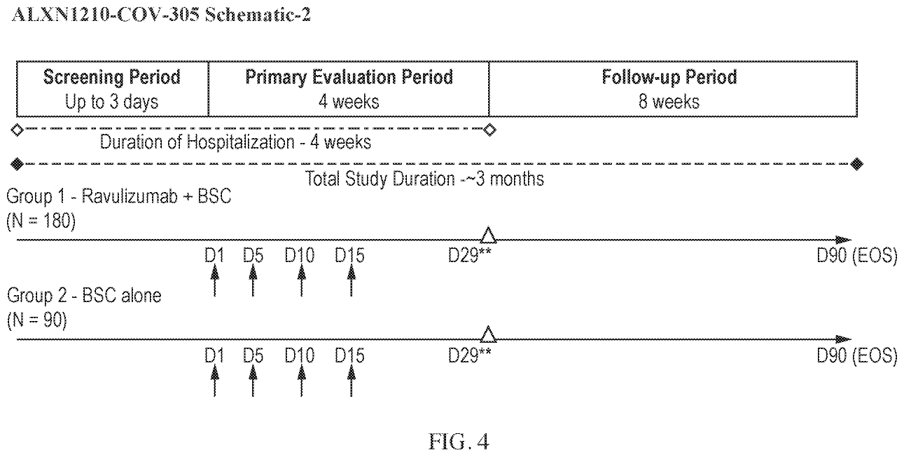

Study ALXN1210-COV-305 is a multicenter Phase 3, open-label, randomized, controlled study designed to evaluate the safety and efficacy of intravenous (IV) ravulizumab plus best supportive care (BSC), compared with BSC alone in patients with a confirmed diagnosis of SARS-CoV-2 infection, and a clinical presentation consistent with COVID-19 severe pneumonia, acute lung injury, or ARDS. A schematic of the trial is set forth in FIG. 4. Patients at least 18 years of age, weighing≥40 kg, and admitted to a designated hospital facility for treatment are screened for eligibility in this study. Accounting for a 10% nonevaluable rate, approximately 270 patients are randomized in a 2:1 ratio (180 patients to receive ravulizumab plus BSC, 90 patients to BSC alone).

Patients randomized to ravulizumab plus BSC receive a weight-based dose of ravulizumab on Day. On Day 5 and Day 10, doses of 600 mg or 900 mg ravulizumab is administered (according to weight category) and on Day 15 patients receive 900 mg ravulizumab. Specifically, a weight based dose is administered on Day 1 as follows: Patients weighing≥40 to <60 kg: 2400 mg/kg; ≥60 to <100 kg: 2700 mg/kg; or ≥100 kg: 3000 mg/kg on Day 1. On Day 5 and Day 10, doses of 600 mg or 900 mg ravulizumab are administered (according to weight category) and on Day 15 patients receive 900 mg ravulizumab. Final assessment is performed at Day 29 or on day of discharge, whichever occurs first. Screening and the Day 1 visits can occur on the same day if the patient has met all inclusion and no exclusion criteria.

Patients in both treatment groups continue to receive medications, therapies, and interventions per standard hospital treatment protocols for the duration of the study.

Approximately 270 patients (180 ravulizumab plus BSC, 90 BSC alone) are randomly assigned to 1 of 2 treatment groups.

The study consists of a Screening Period of up to 3 days, a Primary Evaluation Period of 4 weeks, a final assessment at Day 29 or upon discharge, and a Follow-up Period of 8 weeks. The 2 follow-up visits are conducted 4 weeks apart as a telephone call if the patient is discharged from the hospital or an in-person visit if the patient is still hospitalized. The total duration of each patient's participation is anticipated to be approximately 3 months.

The dosage regimen to be administered during this study is provided in Table 14. No additional doses are allowed during the Primary Evaluation Period (i.e., from Day 1 to Day 29).

TABLE 14
Ravulizumab Dosage Regimen for COVID-19 Severe Pneumonia,
Acute Lung Injury, or Acute Respiratory Distress Syndrome

| Patient Body | | | | |
|---|---|---|---|---|
| Weight (kg)¹ | Day 1 | Day 5 | Day 10 | Day 15 |
| 40 to <60 | 2400 | 600 | 600 | 900 |
| 60 to <100 | 2700 | 900 | 900 | 900 |
| ≥100 | 3000 | 900 | 900 | 900 |

The patient's body weight is recorded on the day of the infusion visit. If the weight at the day of the infusion cannot be obtained, the weight recorded during the previous study visit can be used.

2. Schedule of Activities

The schedule of activities is set forth in Table 15.

TABLE 15
Schedule of Activities

| | Period | | | | | | | | |
|---|---|---|---|---|---|---|---|---|---|
| | Screening | | | | | | | Follow-up | |
| | Period | Primary Evaluation Period | | | | | | Period⁴ | |
| | Study Day (D) | | | | | | | | |
| | D −3 | | | | | | D29 | | |
| | to D −1 | D1^{1,2} | D5² | D10² | D15² | D22 | or ET³ | D60 | D90 |
| | Study Window (days) | | | | | | | | |
| | NA | NA | ±1 | ±1 | ±1 | ±1 | ±1 | ±7 | |
| Eligibility | | | | | | | | | |
| Informed consent | X | | | | | | | | |
| Inclusion/exclusion | X | | | | | | | | |
| Medical history | X | | | | | | | | |
| Demographics | X | | | | | | | | |
| Study Administrative | | | | | | | | | |
| Confirmation of vaccination or | X | | | | | | | X | |
| appropriate antibiotic prophylaxis | | | | | | | | | |
| against N. meningitidis⁵ | | | | | | | | | |
| Screening Laboratory Tests | | | | | | | | | |
| Confirmed positive SARS-COV-2 | X | | | | | | | | |
| test | | | | | | | | | |
| Chest CT or X-ray⁶ | X | | | | | | | | |
| Pregnancy test⁷ | X | | | | | | X | | |
| Randomization | | | | | | | | | |
| Randomized to study drug + BSC | | X | | | | | | | |
| or BSC alone | | | | | | | | | |
| Administration of Study Drug | | | | | | | | | |
| Ravulizumab² | | X | X | X | X | | | | |
| Efficacy Assessments | | | | | | | | | |
| Survival status | | <<monitor continuously>> | | | | | | X | |
| Mechanical ventilation status | | <<monitor continuously>> | | | | | | | |
| Hospitalization status | | <<monitor continuously>> | | | | | | | |
| Intensive care unit status | | <<monitor continuously>> | | | | | | | |
| Dialysis status | | <<monitor continuously>> | | | | | | | |
| FiO2, SpO2, PaO2⁸ | | <<monitor continuously>> | | | | | | | |
| Sequential Organ Failure | X | X | X | X | X | X | X | | |
| Assessment score | | | | | | | | | |
| Safety Assessments | | | | | | | | | |
| Electrocardiogram | X | | | | | | X | | |
| Complete physical examination⁹ | X | | | | | | X | | |
| Abbreviated physical | | X | X | X | X | X | | | |
| examination⁹ | | | | | | | | | |
| Glasgow Coma Scale | X | X | X | X | X | X | X | | |
| Body weight | X | X | X | X | X | | | | |
| Vital sign measurements¹⁰ | X | X | X | X | X | X | X | | |
| Adverse event review and | | <<monitor continuously>> | | | | | | X | |
| evaluation | | | | | | | | | |
| Review safety card¹¹ | | X¹¹ | | | | | X¹¹ | X¹¹ | |
| Safety Laboratory Tests¹² | | | | | | | | | |
| Clinical chemistry | X | X | X | X | X | X | X | | |
| Hematology | X | X | X | X | X | X | X | | |
| Coagulation panel and D-dimer | X | X | X | X | X | X | X | | |
| Urinalysis | X | X | X | X | X | X | X | | |
| Direct Coombs Test | X | | | | | | | | |
| PK/PD/Immunogenicity Tests | | | | | | | | | |
| PK¹³ | | X | X | X | X | X | X | | |
| Total and free C5¹⁴ | | X | X | X | X | X | X | | |
| Immunogenicity¹³ | | X | | | | | X | | |
| Biomarker Tests | | | | | | | | | |
| Serum, urine, and plasma | X | X | X | X | X | X | X | | |
| biomarkers¹⁵ | | | | | | | | | |
| Other | | | | | | | | | |
| Concomitant medication¹⁶ | X | <<monitor continuously>> | | | | | | | |
| Nonpharmacologic treatments | X | <<monitor continuously>> | | | | | | | |
| and therapies¹⁶ | | | | | | | | | |

¹The Day 1 visit may occur on the same day as Screening.
²Patients randomized to ravulizumab plus BSC receive a weight-based dose of ravulizumab on Day 1. On Day 5 and Day 10, additional doses of 600 mg or 900 mg ravulizumab are administered (according to weight category) and on Day 15 patients receive 900 mg of eculizumab.
³The Early Termination Visit is to be conducted when the patient discontinues from the study during the Primary Evaluation Period or upon discharge from the hospital, whichever occurs first.
⁴Additional monitoring is performed during the 2 follow-up visits to review patient status, including survival and pregnancy, and to obtain information about new or worsening TESAEs. The follow-up is conducted as a telephone call if the patient is discharged from the hospital or an in-person visit if the patient is still hospitalized.
⁵Confirmation of meningococcal vaccination within the past 5 years prior to dosing for patients randomized to ravulizumab plus BSC. If vaccination cannot be confirmed, the patient receives prophylactic antibiotics prior to initiating ravulizumab treatment and for at least 8 months from the last infusion of ravulizumab. When patients are vaccinated less than 2 weeks prior to treatment with ravulizumab or after initiation of ravulizumab, they continue antibiotic prophylaxis for at least 2 weeks after meningococcal vaccination.
⁶Can be performed within the 3 days prior to Screening or at Screening. Imaging performed as part of the patient's routine clinical care is expected and acceptable for inclusion in this study.
⁷Urine or serum pregnancy tests (beta human chorionic gonadotropin) is performed in all female patients. A negative pregnancy test result is required before administration of ravulizumab.
⁸SpO2 is measured by pulse oximetry. PaO2 to be measured by arterial blood gas, if available. FiO2 is measured by supplemental oxygen. The highest daily measurement on the lowest inspired supplemental oxygen level is recorded in the CRF/eCRF.
⁹Complete or abbreviated physical examination is to be performed at the timepoints indicated in the Schedule of Assessments. A complete physical examination includes, at a minimum, assessments of the following organs/body systems: skin, head, ears, eyes, nose, throat, neck, lymph nodes, chest, heart, abdomen, extremities, and musculoskeletal. An abbreviated physical examination consists of at least an evaluation of the respiratory and cardiovascular systems. Clinically significant abnormalities or findings are recorded in the AE CRF/eCRF.
¹⁰Vital sign measurements include systolic and diastolic BP (millimeters of mercury [mm Hg]), heart rate (beats/minute), respiratory rate (breaths/minute), and temperature (degrees Celsius [° C.] or degrees Fahrenheit [° F.]). These measurements are taken predose on dosing days.
¹¹When the patient is responsive and capable of understanding, review the Patient Safety Information Card (including discussion of the risks of meningococcal infections) during the hospitalization and at discharge. Upon discharge, patients who received ravulizumab, must carry the Patient Safety Information Card at all times and for at least 8 months after the last infusion of ravulizumab.
¹²Clinical safety laboratory measurements are collected predose on dosing days.
¹³Serum samples for PK and immunogenicity analyses are collected at the timepoints indicated in the SoA for patients randomized to ravulizumab plus BSC. On Day 1/dosing days, immunogenicity and PK samples are collected within 90 minutes before the administration of ravulizumab (predose) and within 60 minutes after the end-of-infusion (postdose). Postdose samples must be collected from a separate line or needle stick to the noninfused arm, not from the infusion line. PK and immunogenicity samples can be collected at any time on nondosing days during the Primary Evaluation Period.
¹⁴Serum samples for total and free C5 analyses are collected at the timepoints indicated in the Schedule of Assessmnets for all patients. For patients randomized to ravulizumab plus BSC, samples are collected within 90 minutes before the administration of ravulizumab (predose) and within 60 minutes after the end-of-infusion (postdose) on dosing days. Postdose samples must be collected from a separate line or needle stick to the noninfused arm, not from the infusion line. Samples can be collected at any time on nondosing days during the Primary Evaluation Period.
¹⁵Serum, plasma, or urine biomarker samples for biomarker analyses are collected at the timepoints indicated in the Schedule of Assessments and stored at the investigational site Samples are collected predose (any time before infusion start).
¹⁶Concomitant medications and nonpharmacologic therapies considered relevant to the treatment of COVID-19 (BSC) or ravulizumab treatment (e.g., antimicrobials, antimalarials, antivirals, steroids, and vasopressors) that the patient is receiving, at the time of Screening and for treating TEAEs/TESAEs, will be recorded in the AE CRF/eCRF.

3. Benefit Assessment

Potential benefits of study participation include: (1) improving survival rate of patients with SARS CoV 2 infection who are receiving ravulizumab plus best supportive care (BSC) compared with BSC alone, (2) decreasing lung injury in patients with SARS CoV 2 infection while on supportive medical care, and (3) improving clinical outcomes in patients with SARS CoV 2 infection while on supportive medical care.

4. Study Population

Patients are eligible to be included in the study only if all the following criteria apply:
1. Patient must be ≥18 years of age at the time of providing informed consent;
2. Confirmed diagnosis of SARS-CoV-2 infection presenting as severe COVID-19 requiring hospitalization;
3. Severe pneumonia, acute lung injury, or ARDS confirmed by computed tomography (CT) or X-ray at Screening or within the 3 days prior to Screening, as part of the patient's routine clinical care;
4. Severe pneumonia, acute lung injury, or ARDS requiring oxygen supplementation with invasive or noninvasive mechanical ventilation (WHO, 2020);
5. Body weight≥40 kg at the time of providing informed consent;
6. Male or female;
7. Female patients of childbearing potential and male patients with female partners of childbearing potential must follow protocol-specified contraception guidance for avoiding pregnancy for 8 months after treatment with the study drug; and
8. Willing and able to provide written informed consent, or with a legally acceptable representative who can provide informed consent, or enrolled under International Council for Harmonisation of Technical Requirements for Pharmaceuticals for Human Use (ICH E6[R2]) 4.8.15 emergency use provisions as deemed necessary by the Investigator.

Patients are excluded from the study if any of the following criteria apply:
1. Patient is not expected to survive for more than 24 hours;
5. Patient is on invasive mechanical ventilation with intubation for more than 48 hours prior to Screening;
6. Severe pre-existing cardiac disease (ie. New York Heart Association Class 3 or Class 4, acute coronary syndrome or persistent ventricular tachyarrhythmias); or
7. Patient has an unresolved Neisseria meningitidis infection.

5. Study Drug

Ravulizumab, a recombinant humanized anti-C5 mAb composed of two 448 amino acid heavy chains and two 214 amino acid light chains, is an IgG2/4 kappa immunoglobulin consisting of human constant regions, and murine complementarity-determining regions grafted onto human framework light- and heavy-chain variable regions. Ravulizumab is produced in Chinese hamster ovarian cell lines and was designed through minimal targeted engineering of eculizumab by introducing 4 unique amino acid substitutions to its heavy chain to extend antibody half-life.

Ravulizumab drug product is supplied for clinical studies as a sterile, preservative-free 10 mg/mL solution in single-use vials and designed for infusion by diluting into commercially available saline (0.9% sodium chloride injection; country-specific pharmacopeia) for administration via IV infusion.

The dosage regimen for the treatment of patients with COVID-19 who are ≥18 years and ≥40 kg and are randomized to ravulizumab plus BSC is presented in Table 16.

TABLE 16
Ravulizumab Dosage Regimen for COVID-19 Severe Pneumonia,
Acute Lung Injury, or Acute Respiratory Distress Syndrome

| Patient Body | | | | |
|---|---|---|---|---|
| Weight (kg)¹ | Day 1 | Day 5 | Day 10 | Day 15 |
| 40 to <60 | 2400 | 600 | 600 | 900 |
| 60 to <100 | 2700 | 900 | 900 | 900 |
| ≥100 | 3000 | 900 | 900 | 900 |

a. The patient's body weight is recorded on the day of the infusion visit. If the weight at the day of the infusion cannot be obtained, the weight recorded during the previous study visit may be used.

Ravulizumab drug product is formulated at pH 7.0 and each 30 mL vial contains 300 mg of ravulizumab, 0.02% polysorbate 80, 150 mM sodium chloride, 6.63 mM sodium phosphate dibasic, 3.34 mM sodium phosphate monobasic, and Water for Injection, United States Pharmacopeia.

The ravulizumab admixture wis administered to the patient using an IV tubing set via an infusion pump followed by an IV flush. Use of a 0.2 micron filter is required during the infusion. The IV flush is infused at the same rate of the infusion and end of flush is considered the end-of-infusion. The IV flush volume is not to be included in the total volume of study drug administered. Additional details are provided in the Pharmacy Manual.

Ravulizumab is manufactured and supplied in single 30 mL vials as a solution concentration of 10 mg/mL (Table 17). Each vial contains 300 mg of ravulizumab for IV administration.

TABLE 17
Ravulizumab Dosage Form and Strength

| | |
|---|---|
| Product Name | Ravulizumab |
| Dosage Form | Concentrate solution for infusion |
| Type | Biologic |
| Dose | 300 mg |
| NIMP and IMP | IMP |
| Route of | Intravenous infusion |
| Administration | |
| Physical Description | 30 mL vial of 10 mg/mL sterile, preservative-free |
| Sourcing | Provided centrally by Alexion Pharmaceuticals or |
| | locally by the study site, subsidiary, or designee |
| Packaging and | Single-use, United States Pharmacopeia/European |
| Labeling | Pharmacopeia Type 1 clear and colorless glass |
| | vial, stoppered with a gray butyl rubber |
| | stopper, and sealed with aluminum seal with a |
| | polypropylene flip-off cap |

Abbreviations: IMP = investigational medicinal product; NIMP = non-investigational medicinal product.

Stability studies of the diluted admixture of ravulizumab (10 mg/mL) in 0.9% sodium chloride injection support an in-use stability of 6 hours at room temperature at 23° C.-27° C. (73° F.-80° F.) and 24 hours when refrigerated at 2° C.-8° C. (36° F.-46° F.). Ravulizumab vials are not frozen or shaken.

6. Concomitant Therapy

Concomitant medications considered relevant to treatment of COVID-19 or ravulizumab treatment (e.g., antimicrobials, antimalarials, antivirals, steroids, and vasopressors) that the patient is receiving at the time of enrollment or receives during the study must be recorded along with: reason for use, dates of administration, including start and end dates, and dosage information including dose and frequency.

Use of the following medications and therapies is prohibited for the specified duration prior to Screening and for the duration of the study: current treatment with a complement inhibitor, rituximab within 3 months of Screening, mitoxantrone within 3 months of Screening, and intravenous immunoglobulin (IVIg) within 3 weeks prior to Screening.

7. Screening Assessments

The SARS-CoV-2 infection is evaluated per the standard diagnostic protocol at the designated hospital. A confirmed positive result is required before randomization. Chest CT or X-ray scans are performed during the Screening Period to confirm findings consistent with severe pneumonia, acute lung injury, or ARDS in patients with COVID-19. Scans performed during the course of the patient's clinical care are accepted and expected to fulfil this diagnostic inclusion criterion for Study.

Urine or serum pregnancy tests (beta human chorionic gonadotropin) are performed in all female patients. A negative pregnancy test result is required before administration of ravulizumab.

8. Efficacy Assessments

The primary efficacy assessment is survival at Day 29.

The following secondary efficacy parameters are also measured through Day 29: (1) mechanical ventilation status, (2) oxygen saturation levels (peripheral capillary oxygen saturation [SpO2], partial pressure of oxygen [PaO2]), (3) supplemental oxygen status (fraction of inspired oxygen [FiO2]), (4) time in the intensive care unit (ICU), (5) duration of hospitalization, and (6) Sequential Organ Failure Assessment (SOFA) score.

Multiple organ failure is a significant indicator of mortality in patients admitted to the ICU. In this study, patients are evaluated using the SOFA score, an assessment tool that includes a review of 6 organ systems: respiratory, renal, hepatic, cardiac, coagulation, and central nervous system (Vincent, 1998). Each organ system is scored from 0 to 4 points using the worst value observed within the previous 24 hours as set forth in Table 18.

TABLE 18
Sequential Organ Failure Assessment Scoring

| Organ | Variable | Score Allocation | | | | |
|---|---|---|---|---|---|---|
| System | (Units) | 0 | 1 | 2 | 3 | 4 |
| Respiratory | PaO2/FiO2 | ≥400 | <400 | <300 | <200 AND | <100 AND |
| | | | | | respiratory | respiratory |
| | | | | | support (e.g., | support |
| | | | | | mechanical | (e.g., |
| | | | | | ventilation) | mechanical |
| | | | | | | ventilation) |
| Renal | Creatinine | <110 | 110-170 | 171-299 | 300-400 | >440 |
| | (μmol/L) | | | | | |
| Hepatic | Bilirubin | <20 | 20-32 | 33-101 | 102-204 | >204 |
| | (μmol/L) | | | | | |
| Cardiac | Inotropes | Mean | Mean | Dopamine < 5 | Dopamine > 5 or | Dopamine > 15 or |
| | (μg/kg/min) | arterial | arterial | or | Epinephrine ≤ 0.1 or | Epinephrine > 0.1 or |
| | | pressure > 70 | pressure < 70 | Dobutamine | Norepinephrine ≤ 0.1 | Norepinephrine > 0.1 |
| | | mmHg | mmHg | any dose | | |
| Coagulation | Platelets | ≥150 | <150 | <100 | <50 | <20 |
| | (× 10³/mm³) | | | | | |
| CNS | Glasgow | 15 | 13-14 | 10-12 | 6-9 | <6 |
| | Coma Scale | | | | | |
| | (GCS) | | | | | |

9. Safety Assessments

The following safety-related parameters are measured through Day 29: (1) body weight and (2) a complete or abbreviated physical examination assessed by the Investigator or designee. A complete physical examination includes, at a minimum, assessments of the skin, head, ears, eyes, nose, throat, neck, lymph nodes, chest, heart, abdomen, extremities, and musculoskeletal. An abbreviated physical examination includes at a minimum, assessment of the respiratory system and cardiovascular systems.

Vital sign measurements include systolic and diastolic blood pressure (millimeters of mercury [mm Hg]), heart rate (HR, beats/minute), respiratory rate (RR, breaths/minute), and temperature (degrees Celsius [° C.] or degrees Fahrenheit [° F.]). Vital sign measurements are taken predose on dosing days

A single 12 lead electrocardiogram (ECG) is conducted to obtain HR, pulse rate (PR), combination of the Q wave, R wave and S wave QRS, interval between the start of the Q wave and the end of the T wave (QT), and corrected QT (QTc) intervals.

The Glasgow Coma Scale (GCS) is a validated prognostic tool used in the clinical assessment of unconsciousness (e.g., patients who are comatose) (Stermbach, 2000). The GCS is comprised of 3 domains—eye response, verbal response, and motor response and within each domain contains a subset of responses that are separately assigned a score as set forth in Table 19. The GCS has also been used in the critical care setting as an aid in managing respiratory support. A total GCS score of <8 is indicative of a patient's need for endotracheal intubation. The GCS is measured to enable calculation of the secondary efficacy endpoint, SOFA score.

TABLE 19
Glasgow Coma Scale

| Response | Score |
|---|---|
| Eye opening | |
| Spontaneous | 4 |
| To speech | 3 |
| To pain | 2 |
| None | 1 |
| Best verbal response | |
| Oriented | 5 |
| Confused conversation | 4 |
| Inappropriate words | 3 |
| Incomprehensible sounds | 2 |
| None | 1 |
| Best motor response | |
| Obeys commands | 6 |
| Localizes pain | 5 |
| Withdrawal (normal flexion) | 4 |
| Abnormal flexion (decorticate) | 3 |
| Extension (decerebrate) | 2 |
| None | 1 |

Source: Sternbach, 2000.

10. Vaccine and Antibiotic Prophylaxis

Patients who have not received a meningococcal vaccination within the past 5 years may be unable to receive meningococcal vaccinations prior to initiating treatment with ravulizumab during this study. If vaccination cannot be confirmed, the patient receives prophylactic antibiotics against meningococcal infection prior to initiating ravulizumab treatment and for at least 8 months from the last infusion of ravulizumab.

When patients can be vaccinated, vaccines against meningococcal serotypes A, C, Y, W135, and B, where available, are recommended to prevent common pathogenic meningococcal serotypes. Patients must be vaccinated or revaccinated according to the current national vaccination guidelines or local practice for vaccination use with complement inhibitors (e.g., ravulizumab). Vaccination may not be sufficient to prevent meningococcal infection. Consideration should be given per official guidance and local practice on the appropriate use of antibacterial agents.

When patients are vaccinated after initiation of ravulizumab, they should continue antibiotic prophylaxis for at least 2 weeks after meningococcal vaccination.

11. Adverse Events and Serious Adverse Events

The definitions of Adverse Events (AEs) and Serious Adverse Events (SAEs) are set forth in Tables 20 and 21, respectively.

TABLE 20
Definition of Adverse Event (AE)
AE Definition

| | |
|---|---|
| 1. | An AE is any untoward medical occurrence in a patient, temporally associated with the |
| | use of study intervention, whether or not considered related to the study intervention. |
| 2. | Note: An AE can therefore be any unfavorable and unintended sign (including an |
| | abnormal laboratory finding), symptom, or disease (new or exacerbated) temporally |
| | associated with the use of study intervention. |

Events Meeting the AE Definition

| | |
|---|---|
| 1. | Any abnormal laboratory test results (hematology, clinical chemistry, or urinalysis) or |
| | other safety assessments (e.g., ECG, radiological scans, vital signs measurements), |
| | including those that worsen from baseline, considered clinically significant in the medical |
| | and scientific judgment of the Investigator (i.e., not related to progression of underlying |
| | disease). |
| 2. | Exacerbation of a chronic or intermittent pre-existing condition including either an increase |
| | in frequency and/or intensity of the condition. |
| 3. | New conditions detected or diagnosed after study intervention administration even though |
| | it may have been present before the start of the study. |
| 4. | Signs, symptoms, or the clinical sequelae of a suspected drug-drug interaction. |
| 5. | Signs, symptoms, or the clinical sequelae of a suspected overdose of either study |
| | intervention or a concomitant medication. Overdose per se will not be reported as an |
| | AE/SAE unless it is an intentional overdose taken with possible suicidal/self-harming |
| | intent. Such overdoses should be reported regardless of sequelac. |
| | “Lack of efficacy” or “failure of expected pharmacological action” per se are not reported |
| | as an AE or SAE. Such instances are captured in the efficacy assessments. However, the |
| | signs, symptoms, and/or clinical sequelae resulting from lack of efficacy are reported as |
| | AE or SAE if they fulfill the definition of an AE or SAE. |

Events Not Meeting the AE Definition

| | |
|---|---|
| 6. | Medical or surgical procedure (e.g., endoscopy, appendectomy): The condition that leads |
| | to the procedure is the AE. Situations in which an untoward medical occurrence did not |
| | occur (e.g., hospitalization for elective surgery if planned before the signing the ICF, |
| | admissions for social reasons or for convenience). |
| 7. | Anticipated day-to-day fluctuations of pre-existing disease(s) or condition(s) present or |
| | detected at the start of the study that do not worsen. |
| 8. | A medication error (including intentional misuse, abuse, and overdose of the product) or |
| | use other than what is defined in the protocol is not considered an AE unless there is an |
| | untoward medical occurrence as a result of a medication error. |
| 9. | Cases of pregnancy that occur during maternal or paternal exposure to study intervention |
| | are to be reported within 24 hours of Investigator/site awareness. Data on fetal outcome |
| | and breastfeeding is collected for regulatory reporting and safety evaluation. |
| 10. | Any clinically significant abnormal laboratory findings or other abnormal safety |
| | assessments which are associated with the underlying disease, unless judged by the |
| | Investigator to be more severe than expected for the patient's condition. |
| 11. | The disease/disorder being studied or expected progression, signs, or symptoms of the |
| | disease/disorder being studied, unless more severe than expected for the patient's |
| | condition. |
| 12. | Situations in which an untoward medical occurrence did not occur (social and/or |
| | convenience admission to a hospital). |

TABLE 21
Definition of Serious Adverse Event (SAE)
An SAE is defined as any untoward medical occurrence that, at any dose:

Results in death
Is life-threatening
The term “life-threatening” in the definition of “serious” refers to an event in which the
patient was at risk of death at the time of the event. It does not refer to an event, which
hypothetically might have caused death, if it was more severe.
Requires inpatient hospitalization or prolongation of existing hospitalization
In general, hospitalization signifies that the patient has been detained (usually involving at
least an overnight stay) at the hospital or emergency ward for observation and/or treatment
that would not have been appropriate in the physician's office or outpatient setting.
Complications that occur during hospitalization are AEs. If a complication prolongs
hospitalization or fulfills any other serious criteria, the event is serious. When in doubt as to
whether “hospitalization” occurred or was necessary, the AE should be considered serious.
Hospitalization for elective treatment of a pre-existing condition that did not worsen from
baseline is not considered an AE.
Results in persistent disability/incapacity
The term disability means a substantial disruption of a person's ability to conduct normal life
functions.
This definition is not intended to include experiences of relatively minor medical
significance such as uncomplicated headache, nausea, vomiting, diarrhea, influenza, and
accidental trauma (e.g., sprained ankle) which may interfere with or prevent everyday life
functions but do not constitute a substantial disruption.
Is a congenital anomaly/birth defect
Other situations:
Medical or scientific judgment should be exercised in deciding whether SAE reporting is
appropriate in other situations such as important medical events that may not be immediately
life-threatening or result in death or hospitalization but may jeopardize the patient or may
require medical or surgical intervention to prevent one of the other outcomes listed in the
above definition. These events should usually be considered serious.
Examples of such events include invasive or malignant cancers, intensive treatment in an
emergency room or at home for allergic bronchospasm, blood dyscrasias or convulsions that
do not result in hospitalization, or development of drug dependency or drug abuse.

All AEs are reported to the Investigator or qualified designee by the patient (or, when appropriate, by a caregiver, surrogate, or the patient's legally acceptable representative). All AEs and SAEs are collected from the time of informed consent until through the timepoints specified in the Schedule of Assessments.

12. Pharmacokinetics, Pharmacodynamics, and Biomarkers

Samples are collected as specified in the Schedule of Assessments to determine serum concentrations of ravulizumab. The actual date and time (24-hour clock time) of each sample is recorded.

Samples are collected as specified in the Schedule of Assessments to assess the effect of ravulizumab on total and free C5. The actual date and time (24-hour clock time) of each sample is recorded.

Serum, plasma, or urine samples are collected for biomarker analysis to evaluate complement activation and related pathways and cardiovascular health, and their clinical response to ravulizumab. These biomarkers include complement pathway proteins (e.g., total and free C5, soluble C5b-9 [sC5b-9], C5a, C3a, total C3, Factor B and Ba), cytokines associated with inflammation and disease (e.g, IL-1, IL-6, IL-8, IL-21, tumor necrosis factor [TNF]-b, and monocyte chemoattractant protein [MCP]-1), and markers associated with cardiovascular disease (procalcitonin, myoglobin, high sensitivity troponin I [hs-TnI] and N-terminal pro b-type natriuretic peptide [NT-proBNP]).

Antibodies to ALXN1210 (i.e., antidrug antibody [ADA]) are evaluated in serum samples collected from all patients according to the Schedule of Assessments. Additionally, serum samples are collected at the final visit from patients who discontinued ravulizumab or were withdrawn from the study. Serum samples are screened for antibodies binding to ravulizumab and the titer of confirmed positive samples are reported. Other analyses can be performed to further characterize the immunogenicity of ravulizumab.

The detection and characterization of antibodies to ravulizumab is performed using a validated assay method. Samples collected for detection of antibodies to ravulizumab are also evaluated for study intervention serum concentration to enable interpretation of the antibody data. Confirmed antibody positive samples are further evaluated for antibody titer and the presence of neutralizing antibodies.

Blood samples are collected for biomarker analyses and the data may be used for future exploratory research related to complement activation and inflammatory processes. The samples can also be used to develop tests/assays, including diagnostic tests related to C5 inhibitors and COVID 19 with clinical presentation of severe pneumonia, acute lung injury, or ARDS. The samples can be analyzed as part of a multi-study assessment of biomarkers in the response to ravulizumab to understand COVID 19 or related conditions.

13. Statistical Considerations

The primary null hypothesis is that there is no difference in survival between ravulizumab plus BSC and BSC alone as measured by the difference in the proportions surviving at Day 29 between the 2 treatment groups. The alternative hypothesis is that ravulizumab plus BSC improves survival at Day 29 compared with BSC alone.

The null hypotheses associated with the secondary objectives are that ravulizumab plus BSC is no different than BSC alone for the respective endpoints. The alternative hypotheses are described below:
1. Number of days free of mechanical ventilation: The alternative hypothesis is that treatment with ravulizumab plus BSC increases the number days free of mechanical ventilation at Day 29 compared with BSC alone.
2. Change in SpO2/FiO2: The alternative hypothesis is that treatment with ravulizumab plus BSC improves changes in SpO2/FiO2 at Day 29 compared with BSC alone.
3. Duration of ICU stay: The alternative hypothesis is that treatment with ravulizumab plus BSC reduces the number days in the ICU at Day 29 compared with BSC alone.
4. Change in SOFA score: The alternative hypothesis is that treatment with ravulizumab plus BSC improves changes in SOFA score at Day 29 compared with BSC alone.
5. Duration of hospitalization: The alternative hypothesis is that treatment with ravulizumab plus BSC reduces the number days in the hospital at Day 29 compared with BSC alone.

A sample size of 243 patients (162 ravulizumab plus BSC, 81 BSC alone) is required to ensure at least 90% power and detect an improvement in survival from 60% in the BSC alone group to 80% in the ravulizumab plus BSC group at Day 29. This sample size calculation assumes: (1) 1-sided Z-test of the difference in 2 proportions, (2) type I error=0.025, (3) pooled variance, (4) 2:1 randomization on the 2 treatment groups, and (5) one interim analysis at 50% information which is after collecting primary efficacy data on approximately 122 patients. The early stopping boundaries for efficacy and futility are constructed using a spending function as Lan DeMets spending function with O'Brien Fleming flavor and p spending function as Gamma(4) (Lan, 1983; Hwang, 1990).

Considering a nonevaluable rate of 10%, this study is planned to randomize approximately 270 patients (180 ravulizumab+BSC, 90 BSC alone). The population sets used for analysis sets are defined in Table 22.

TABLE 22
Analysis Sets

| Analysis Set | Description |
|---|---|
| Full Analysis | The FAS consists of all randomized patients who receive at least |
| Set (FAS) | 1 dose of ravulizumab for patients randomized to |
| | ravulizumab plus BSC or who were randomized to BSC alone. |
| | The FAS will be used for the analysis of efficacy data and is |
| | considered the primary analysis population. |
| Per-Protocol | The PPS is a subset of the FAS without any important protocol |
| Set (PPS) | deviations that could impact efficacy analyses. Determination of |
| | applicable important protocol deviations for this purpose will be |
| | made prior to database lock. |
| | The PPS will be used for sensitivity analyses of the primary and |
| | secondary efficacy endpoints. |
| Safety Set (SS) | The SS is identical to the FAS and consists of all randomized |
| | patients who receive at least 1 dose of ravulizumab for patients |
| | randomized to ravulizumab plus BSC or who were randomized |
| | to BSC alone. The SS will be used for the analysis of safety data. |

The primary analysis is conducted when all patients have completed the Primary Evaluation Period. This analysis includes all efficacy, safety, and PK/PD/immunogenicity study data for regulatory submission purposes and is the final analysis of the Primary Evaluation Period.

Summary statistics are presented by treatment group and by visit, where applicable. Descriptive statistics for continuous variables minimally include the number of patients, mean, standard deviation, median, minimum, and maximum. For categorical variables, frequencies and percentages are presented. Graphical displays are provided as appropriate. All statistical analyses are performed based on a 2-sided Type I error of 5%, unless otherwise noted.

Baseline represents assessments/procedures that are performed on or before the infusion of ravulizumab on Day 1 (for patients randomized to ravulizumab plus BSC) and on or before initiation of assessments/procedures on Day 1 (for patients randomized to BSC alone).

Analyses are performed using SAS® software Version 9.4 or higher. The primary efficacy endpoint is survival (based on all-cause mortality) at Day 29 and is compared between the 2 treatment groups using a 1-sided Z-test of the difference in 2 proportions with a pooled variance and a Type I error of 0.025. The estimated risk difference is summarized along with the 95% confidence interval. If a patient is discharged before Day 29, he/she is considered as survived at Day 29.

Survival is also analyzed using the method of Kaplan and Meier (KM) and compared using a log-rank test as a sensitivity analysis. Hazard ratio and risk reduction are summarized from a Cox proportional hazards model. Confidence intervals (95%) are presented for the survival estimate at Day 29 based on the complementary log-log transformation. Kaplan-Meier curves for both treatment groups are produced.

A sensitivity analysis of the primary endpoint is also performed using a 3-level categorical outcome of 3) alive and discharged from the ICU; 2) alive and in the ICU or 1) death. The 2 treatment groups is compared using a chi-squared test.

Additional sensitivity analyses include statistical models adjusting for age, randomization stratification factor, and other important covariates. The Statistical Analysis Plan (SAP) describes the sensitivity analyses in greater detail.

An interim analysis of the primary endpoint is also conducted.

Number of days free of mechanical ventilation at Day 29 is compared between treatment groups using an analysis of covariance (ANCOVA), adjusting for age, and randomization stratification factor, among survivors. If a patient is discharged from the hospital prior to Day 29, he/she is considered alive and free of mechanical ventilation for the remaining days up to Day 29.

Change from baseline in SpO2/FiO2 at Day 29 is analyzed using a mixed model for repeated measures (MMRM) with baseline SpO2/FiO2, age, randomization stratification factor, treatment group indicator, study day, and study day by treatment group interaction as covariates. All patients who survive to Day 29 are included in the model, except those patients without any postbaseline scores. Sensitivity analyses includes imputations for missing data. Change from baseline in PaO2/FiO2 at Day 29 is also analyzed using a MMRM with baseline PaO2/FiO2, age, randomization stratification factor, treatment group indicator, study day, and study day by treatment group interaction as fixed covariates. All patients with PaO2/FiO2 data who survive to Day 29 are included in the model, except those without any postbaseline scores. Sensitivity analyses include imputations for missing data. Changes from baseline in SpO2/FiO2 and PaO2/FiO2 are also summarized for non-survivors.

Duration of ICU stay at Day 29 is compared between treatment groups using an ANCOVA, adjusting for age and randomization stratification factor, among survivors. Duration of ICU stay at Day 29 is also summarized for non-survivors.

Change from baseline in SOFA score at Day 29 is analyzed in a similar manner as change from baseline in SpO2/FiO2, using an MMRM and including baseline SOFA score. Duration of hospitalization at Day 29 is analyzed in a similar manner as duration of ICU stay.

A closed testing procedure is applied to control the type I error for the analyses of the primary and secondary endpoints. If the primary endpoint is statistically significant in favor of ravulizumab, the secondary endpoints are evaluated according to the following rank order:
1. Number of days free of mechanical ventilation at Day 29,
2. Change from baseline in SpO2/FiO2 at Day 29,
3. Duration of ICU stay at Day 29,
4. Change from baseline in SOFA score at Day 29, and
5. Duration of hospitalization at Day 29.

The hypothesis testing proceeds from highest rank (#1) the number of days free of mechanical ventilation at Day 29 to the lowest rank (#5) duration of hospitalization at Day 29, and if statistical significance is not achieved at an endpoint (p≥0.05), then endpoints of lower rank are not considered to be statistically significant. Confidence intervals and p-values are presented for all secondary efficacy endpoints for descriptive purposes, regardless of the outcome of the closed testing procedure.

All safety analyses are made on the Safety Set (SS). Safety results are reported by treatment group.

The analysis and reporting of AEs and SAEs is based on TEAEs and TESAEs, defined as AEs and SAEs with onset during or after treatment with ravulizumab. The incidence of TEAEs and TESAEs is summarized by System Organ Class and Preferred Term, with additional summaries showing relationship to ravulizumab, severity, TEAEs or TESAEs leading to ravulizumab discontinuation, and TESAEs resulting in death.

Laboratory measurements, as well as their changes from baseline at each visit and shift from baseline, if applicable, are summarized. Protocol-required laboratory assessments are set forth in Table 23.

TABLE 23
Protocol-required Laboratory Assessments
The following parameters may be analyzed by the local laboratory:

| | |
|---|---|
| Hematology Panel | Clinical Chemistry Panel |
| Haptoglobin | Alanine aminotransferase |
| Hematocrit | Aspartate aminotransferase |
| Hemoglobin | Blood urea nitrogen |
| Platelet count | C-reactive protein |
| WBC count | Creatinine |
| WBC differential | Lactate dehydrogenase |
| Coagulation Panel | Sodium |
| D-dimer | Total bilirubin (direct and indirect) |
| International normalized ratio | Ferritin |
| Partial thromboplastin time | Other |
| Prothrombin time | Arterial blood gas |
| Fibrinogen | Beta human chorionic gonadotropin (all females |
| Urinalysis | of childbearing potential only) |
| Albumin | Direct Coombs test |
| Creatinine | |
| Protein | |

The following parameters may be analyzed by the central laboratory:

| | |
|---|---|
| Inflammatory Biomarkers | Other Biomarkers |
| IL-1, IL-6, IL-8, and IL-21 | MCP-1 |
| Complement Pathway Biomarkers | TNF-b |
| Total and free C5, sC5b-9, C5a, C3a, total C3, | Immunogenicity assay |
| Factor B, and Factor Ba | Pharmacokinetic assay |
| Cardiac Biomarkers | |
| Procalcitonin | |
| Myoglobin | |
| NT-proBNP | |
| hs-TnI | |

Vital sign measurements and physical examination findings are also summarized over time.

Individual serum concentration data for all patients who receive at least 1 dose of ravulizumab and who have evaluable PK/PD data are used to summarize PK/PD parameters for ravulizumab. Descriptive statistics are presented for all ravulizumab PK/PD endpoints at each sampling time. The PD effects of ravulizumab are summarized using absolute values and changes and percentage changes from baseline in free C5 serum concentrations over time, as appropriate.

Serum, urine, and plasma biomarkers' actual values, and changes from baseline, and their association with observed clinical responses to ravulizumab are summarized over time, as appropriate.

The incidence and titers for ADAs to ravulizumab are summarized in tabular format by treatment group. The proportion of patients ever positive and the proportion of patients always negative may be explored. Confirmed ADA positive samples are evaluated for the presence of neutralizing antibodies.

Survival (based on all-cause mortality) at Day 60 and Day 90 is estimated using the KM method and compared using a log-rank test. Hazard ratio and risk reduction are summarized from a Cox proportional hazards model. Confidence intervals (95%) are presented for the survival estimates at Days 60 and 90 based on the complementary log-log transformation. KM curves for both treatment groups are produced.

Incidence of progression to renal failure requiring dialysis at Day 29 re analyzed in a similar manner as the primary endpoint.

An interim analysis for efficacy and futility is conducted when approximately 122 patients have completed Day 29 (or were ET). If the stopping criteria are met, the study may be terminated early for efficacy or futility depending on which stopping boundary is crossed. The early stopping boundaries for efficacy and futility is constructed using α-spending function as Lan-DeMets (O'Brien-Fleming) spending function and s-spending function as Gamma(-4). A 1-sided Z-test of the difference in 2 proportions will used with a pooled variance and a type I error of 0.025.

The SAP describes the planned interim analyses in greater detail.

Provided the study was not stopped early for efficacy or futility, the final primary analysis is conducted when all patients have completed the Primary Evaluation Period. This analysis includes all efficacy, safety, and PK/PD/immunogenicity study data for regulatory submission purposes.

### Example 9: Phase 3 Clinical Trial Comparing Ravulizumab Against Best Supportive Care (BSC) in Patients with COVID 19 Severe Pneumonia, Acute Lung Injury, or Acute Respiratory Distress Syndrome

A phase 3, open-label, randomized, controlled study (“ALXN1210-COV-305”) is conducted to evaluate the efficacy, safety, pharmacokinetics, and pharmacodynamics of intravenously administered ravulizumab compared with best supportive care in patients with Coronavirus Disease 2019 (COVID-19) severe pneumonia, acute lung injury, or acute respiratory distress syndrome. Patients are randomly assigned to receive ravulizumab in addition to best supportive care (BSC) (⅔ of the patients) or BSC alone (⅓ of the patients). Best supportive care consists of medical treatment and/or medical interventions per routine hospital practice.

In the ravulizumab plus best supportive care arm of the study, weight-based doses of ravulizumab (also known as ULTOMIRIS and ALXN1210) are administered intravenously on Days 1, 5, 10, and 15. Patients in this arm of the study also receive medications, therapies, and interventions per standard hospital treatment protocols. In the best supportive care arm of the study patients receive medications, therapies, and interventions per standard hospital treatment protocols.

1. Objectives

The primary objective of the study is to evaluate the effect of ravulizumab and best supportive care compared with best supportive care alone on the survival of patients with COVID 19 (e.g., survival (based on all cause mortality) at Day 29). The primary outcome measure is survival (based on all-cause mortality) at Day 29.

The secondary objective of the study is to evaluate the efficacy of ravulizumab plus best supportive care compared with best supportive care alone on outcomes in patients with COVID 19. Secondary outcome measures include (1) number of days free of mechanical ventilation at Day 29, (2) duration of intensive care unit stay at Day 29, (3) change from baseline in sequential organ failure assessment at Day 29, (4) change from baseline in SpO2/FiO2 at Day 29, (5) duration of hospitalization at Day 29, and (5) survival (based on all-cause mortality) at Day 60 and Day 90.

The safety objective is to characterize the overall safety of ravulizumab plus best supportive care compared with best supportive care alone in patients with COVID 19 (e.g., as assessed by incidence of treatment emergent adverse events (TEAEs) and treatment emergent serious adverse events (TESAEs).

A further objective is to characterize the pharmacokinetic/pharmacodynamic and immunogenicity of ravulizumab in patients with COVID 19 (e.g., as assessed by change in serum ravulizumab concentrations over time, change in serum free and total C5 concentrations over time, and incidence and titer of anti ALXN1210 antibodies).

With respect to biomarkers, the objective is to assess the effect of C5 inhibition on systemic activation of complement, inflammation, and prothrombic activity in patients with COVID 19 (e.g., as assessed by change in absolute levels of soluble biomarkers in blood associated with complement activation, inflammatory processes, and hypercoagulable states over time).

Exploratory objectives include (1) evaluating the effect of ravulizumab and BSC compared with BSC alone on progression to renal failure requiring dialysis in patients with COVID 19 (e.g., as assessed by incidence of progression to renal failure requiring dialysis at Day 29), (2) evaluating the effect of ravulizumab plus BSC compared with BSC alone on clinical improvement in patients with COVID 19 (e.g., as assessed by time to clinical improvement (based on a modified 6 category ordinal scale) over 29 days) and (3) evaluating the effect of ravulizumab plus BSC compared with BSC alone on the health related quality of life of patients with COVID 19 (e.g., as assessed by (a) SF 12 PCS and MCS scores at Day 29 (or discharge), Day 60, and Day 90 and (b) EuroQol 5 dimension 5 level (EQ-5D-5L) scores at Day 29 (or discharge), Day 60, and Day 90).

Baseline is defined as the last available assessment on or before Day 1 for all patients. Day 1 is be defined as the date of the first infusion of ravulizumab for patients randomized and dosed with ravulizumab and as the date of randomization for patients randomized, but not dosed with ravulizumab.

2. Overall Design

Study ALXN1210-COV-305 is a multicenter Phase 3, open-label, randomized, controlled study designed to evaluate the safety and efficacy of intravenous (IV) ravulizumab plus best supportive care (BSC), compared with BSC alone in patients with a confirmed diagnosis of SARS-CoV-2 infection, and a clinical presentation consistent with COVID-19 severe pneumonia, acute lung injury, or ARDS. Patients at least 18 years of age, weighing≥40 kg, and admitted to a designated hospital facility for treatment are screened for eligibility in this study. Accounting for a 10% nonevaluable rate, approximately 270 patients are randomized in a 2:1 ratio (180 patients to receive ravulizumab plus BSC, 90 patients to BSC alone).

Patients randomized to ravulizumab plus BSC receive a weight-based dose of ravulizumab on Day 1. On Day 5 and Day 10, doses of 600 mg or 900 mg ravulizumab is administered (according to weight category) and on Day 15 patients receive 900 mg ravulizumab. Patients in both treatment groups continue to receive medications, therapies, and interventions per standard hospital treatment protocols for the duration of the study.

Screening and the Day 1 visits can occur on the same day if the patient has met all inclusion and no exclusion criteria.

Approximately 270 patients (180 ravulizumab+BSC, 90 BSC alone) are randomly assigned to 1 of 2 treatment groups.

The study consists of a Screening Period of up to 3 days, a Primary Evaluation Period of 4 weeks, a final assessment at Day 29, and a Follow-up Period of 8 weeks. The 2 follow-up visits are conducted 4 weeks apart as a telephone call if the patient is discharged from the hospital or an in-person visit if the patient is still hospitalized. The total duration of each patient's participation is anticipated to be approximately 3 months.

The dosage regimen to be administered during this study is provided in Table 24. Specifically, a weight based dose of ravulizumab is administered on Day 1 as follows: Patients weighing≥40 to <60 kg: 2400 mg; ≥60 to <100 kg: 2700 mg; or ≥100 kg: 3000 mg. A weight based dose of ravulizumab is administered on Day 5 and Day 10 as follows: Patients weighing≥40 to <60 kg: 600 mg; ≥60 to <100 kg: 900 mg; or ≥100 kg: 900 mg. On Day 15, patients receive 900 mg ravulizumab. No additional doses are allowed during the Primary Evaluation Period (i.e., from Day 1 to Day 29).

TABLE 24
Ravulizumab Dosage Regimen for COVID-19 Severe Pneumonia,
Acute Lung Injury, or Acute Respiratory Distress Syndrome

| Patient Body | Day 1 | Day 5 | Day 10 | Day 15 |
|---|---|---|---|---|
| Weight (kg)¹ | Dose (mg) | Dose (mg) | Dose (mg) | Dose (mg) |
| 40 to <60 | 2400 | 600 | 600 | 900 |
| 60 to <100 | 2700 | 900 | 900 | 900 |
| ≥100 | 3000 | 900 | 900 | 900 |

A. The patient's body weight is recorded on the day of the infusion visit. If the weight at the day of the infusion cannot be obtained, the weight recorded during the previous study visit may be used.

3. Schedule of Activities

The schedule of activities is set forth in Table 25.

TABLE 25
Schedule of Activities

| | Period | | | | | | | | | |
|---|---|---|---|---|---|---|---|---|---|---|
| | Screening | | | | | | | | Follow-up | |
| | Period | Primary Evaluation Period | | | | | | | Period⁵ | |
| | Study Day (D) | | | | | | | | | |
| | D −3 | | | | | | D29/ | Early | | |
| | to D −1 | D1^{1,2} | D5² | D10² | D15² | D22 | Discharge³ | Termination⁴ | D60 | D90 |
| | Study Window (days) | | | | | | | | | |
| | NA | NA | ±1 | ±1 | ±1 | ±1 | ±1 | | ±7 | |
| Eligibility | | | | | | | | | | |
| Informed consent | X | | | | | | | | | |
| Inclusion/exclusion | X | | | | | | | | | |
| Medical history¹⁹ | X | | | | | | | | | |
| Demographics | X | | | | | | | | | |
| Study Administrative | | | | | | | | | | |
| Confirmation of | X | | | | | | | | X | |
| vaccination or appropriate | | | | | | | | | | |
| antibiotic prophylaxis | | | | | | | | | | |
| against N. meningitidis⁶ | | | | | | | | | | |
| Screening Laboratory Tests | | | | | | | | | | |
| Confirmed positive | X | | | | | | | | | |
| SARS-COV-2 test | | | | | | | | | | |
| Chest CT or X-ray⁷ | X | | | | | | | | | |
| Pregnancy test⁸ | X | | | | | | X | X | | |
| Randomization | | | | | | | | | | |
| Randomized to study | | X | | | | | | | | |
| drug + BSC or BSC alone | | | | | | | | | | |
| Administration of Study | | | | | | | | | | |
| Drug | | | | | | | | | | |
| Ravulizumab² | | X | X | X | X | | | | | |
| Efficacy Assessments | | | | | | | | | | |
| Survival status | | <<monitor continuously>>¹⁸ | | | | | | | X | |
| Mechanical ventilation | X | <<monitor continuously>>¹⁸ | | | | | | | | |
| status | | | | | | | | | | |
| Hospitalization status | | <<monitor continuously>>¹⁸ | | | | | | | | |
| Intensive care unit status | | <<monitor continuously>>¹⁸ | | | | | | | | |
| Dialysis status | | <<monitor continuously>>¹⁸ | | | | | | | | |
| FiO2, SpO2, PaO2⁹ | | << monitor daily>>⁹ | | | | | | | | |
| Sequential Organ Failure | X | X | X | X | X | X | X | X | | |
| Assessment score | | | | | | | | | | |
| Safety Assessments | | | | | | | | | | |
| Electrocardiogram | X | | | | | | X | X | | |
| Complete physical | X | | | | | | X | X | | |
| examination¹⁰ | | | | | | | | | | |
| Abbreviated physical | | X | X | X | X | X | | | | |
| examination¹⁰ | | | | | | | | | | |
| Glasgow Coma Scale | X | X | X | X | X | X | X | X | | |
| Body weight | X | X | X | X | X | | | | | |
| Vital sign measurements | X | X | X | X | X | X | X | X | | |
| (predose)¹¹ | | | | | | | | | | |
| Adverse event review and | X | <<monitor continuously>> | | | | | | | X | |
| evaluation | | | | | | | | | | |
| Review safety card¹² | | X¹² | | | | | X¹² | X¹² | X¹² | |
| Safety Laboratory Tests | | | | | | | | | | |
| (Predose)¹³ | | | | | | | | | | |
| Clinical chemistry | X | X | X | X | X | X | X | X | | |
| Hematology | X | X | X | X | X | X | X | X | | |
| Coagulation panel and D- | X | X | X | X | X | X | X | X | | |
| dimer | | | | | | | | | | |
| Urinalysis | X | X | X | X | X | X | X | X | | |
| Direct Coombs test | X | | | | | | | | | |
| PK/PD/Immunogenicity | | | | | | | | | | |
| Tests | | | | | | | | | | |
| PK¹⁴ | | X | X | X | X | X | X | X | | |
| Total and free C5¹⁵ | | X | X | X | X | X | X | X | | |
| Immunogenicity | | X | | | | | X | X | | |
| (predose)¹⁴ | | | | | | | | | | |
| Biomarker Tests | | | | | | | | | | |
| Serum and plasma | X | X | X | X | X | X | X | X | | |
| biomarkers (predose)¹⁶ | | | | | | | | | | |
| Other | | | | | | | | | | |
| Concomitant medication¹⁷ | X | <<monitor continuously>> | | | | | | | X | |
| Nonpharmacologic | X | <<monitor continuously>> | | | | | | | X | |
| treatments and therapies¹⁸ | | | | | | | | | | |
| Patient-reported Outcomes | | | | | | | | | | |

| | | |
|---|---|---|
| SF-12 | X | X |
| EQ-5D-5L | X | X |

¹The Day 1 visit may occur on the same day as Screening.
²Patients randomized to ravulizumab plus BSC receive a weight-based dose of ravulizumab on Day 1. On Day 5 and Day 10, additional doses of 600 mg or 900 mg ravulizumab are administered (according to weight category) and on Day 15 patients receive 900 mg of ravulizumab.
³If a patient is discharged before the end of the Primary Evaluation Period (Day 29), the patient undergoes the early discharge assessments. In addition, the patient is contacted via telephone on Day 29 to assess health status (e.g., survival, mechanical ventilation, hospitalization, intensive care unit, and dialysis). Follow-up telephone calls should be performed as planned unless the patient withdraws consent.
⁴The Early Termination Visit is to be conducted when the patient discontinues from the study during the Primary Evaluation Period. In addition, the patient is contacted via telephone on Day 29 to assess health status (e.g., survival, mechanical ventilation, hospitalization, intensive care unit, and dialysis).
⁵Additional monitoring is performed during the 2 follow-up visits to review patient status, including survival and pregnancy, and to obtain information about new or worsening TESAEs. The follow-up is conducted as a telephone call if the patient is discharged from the hospital or an in-person visit if the patient is still hospitalized.
⁶Confirmation of meningococcal vaccination within the past 5 years prior to dosing for patients randomized to ravulizumab plus BSC. If vaccination cannot be confirmed, the patient should receive prophylactic antibiotics prior to initiating ravulizumab treatment and for at least 8 months from the last infusion of ravulizumab. When patients are vaccinated less than 2 weeks prior to treatment with ravulizumab or after initiation of ravulizumab, they should continue antibiotic prophylaxis for at least 2 weeks after meningococcal vaccination.
⁷Can be performed within the 3 days prior to Screening or at Screening. Imaging performed as part of the patient's routine clinical care is expected and acceptable for inclusion in this study.
⁸Urine or serum pregnancy tests (beta human chorionic gonadotropin) to be performed in all female patients. A negative pregnancy test result is required before administration of ravulizumab.
⁹SpO2 is measured by pulse oximetry. PaO2 is measured by arterial blood gas, if available. FiO2 to be measured by supplemental oxygen. For patients treated with ravulizumab, SpO2, PaO2 (if available), and FiO2 are measured predose on Day 1. The highest daily measurement of oxygen pressure or saturation on the lowest inspired supplemental oxygen level is recorded in the CRF/eCRF.
¹⁰Complete or abbreviated physical examination is to be performed at the timepoints indicated in the Schedule of Assessments. A complete physical examination includes, at a minimum, assessments of the following organs/body systems: skin, head, ears, eyes, nose, throat, neck, lymph nodes, chest, heart, abdomen, extremities, and musculoskeletal. An abbreviated physical examination consists of at least an evaluation of the respiratory and cardiovascular systems. Clinically significant abnormalities or findings will be recorded in the AE CRF/eCRF.
¹¹Vital sign measurements include systolic and diastolic BP (millimeters of mercury [mm Hg]), heart rate (beats/minute), respiratory rate (breaths/minute), and temperature (degrees Celsius [° C.] or degrees Fahrenheit [° F.]). These measurements are taken predose on dosing days.
¹²When the patient is responsive and capable of understanding, review the Patient Safety Information Card (including discussion of the risks of meningococcal infections) during the hospitalization and at discharge. Upon discharge, patients who received ravulizumab, must carry the Patient Safety Information Card at all times and for at least 8 months after the last infusion of ravulizumab.
¹³Clinical safety laboratory measurements are collected predose on dosing days.
¹⁴Serum samples for PK and immunogenicity analyses are collected at the timepoints indicated in the Schedule of Assessmnets for patients randomized to ravulizumab plus BSC. On Day 1/dosing days, immunogenicity and PK samples are collected within 4 hours before the administration of ravulizumab (predose) and PK samples are collected within 4 hours after the end-of-infusion (postdose). Postdose PK samples must be collected from a separate line or needle stick to the noninfused arm, not from the infusion line. Pharmacokinetic and immunogenicity samples can be collected at any time on nondosing days during the Primary Evaluation Period.
¹⁵Serum samples for total and free C5 analyses are collected at the timepoints indicated in the Schedule of Assessments for all patients. For patients randomized to ravulizumab plus BSC, samples are collected within 4 hours before the administration of ravulizumab (predose) and within 4 hours after the end-of-infusion (postdose) on dosing days. Postdose samples must be collected from a separate line or needle stick to the noninfused arm, not from the infusion line. Samples can be collected at any time on nondosing days during the Primary Evaluation Period.
¹⁶Serum and plasma biomarker samples for biomarker analyses are collected for all patients at the timepoints indicated in the Schedule of Assessments and stored. Samples are collected predose (any time before infusion start) for patients who are randomized to the ravulizumab plus BSC treatment group.
¹⁷Concomitant medications and nonpharmacologic therapies considered relevant to the treatment of COVID-19 (BSC) or ravulizumab treatment (e.g., antimicrobials, antimalarials, antivirals, steroids, and vasopressors) that the patient is receiving, at the time of Screening and for treating TEAEs/TESAEs, are recorded in the AE CRF/eCRF.
¹⁸Assessed via a telephone call at Day 29 for all patients who are discharged before the end of the Primary Evaluation Period (Day 29).
¹⁹Medical history includes date of first onset of signs and symptoms of SARS-COV-2 infection.

4. Benefit Assessment

Potential benefits of study participation include: (1) improving survival rate of patients with SARS CoV 2 infection who are receiving ravulizumab+best supportive care (BSC) compared with BSC alone, (2) decreasing lung injury in patients with SARS CoV 2 infection while on supportive medical care, and (3) improving clinical outcomes in patients with SARS CoV 2 infection while on supportive medical care.

5. Inclusion and Exclusion Criteria

Patients are eligible to be included in the study only if all the following criteria apply:
1. Patient must be ≥18 years of age at the time of providing informed consent;
2. Confirmed diagnosis of SARS-CoV-2 infection (e.g., via polymerase chain reaction [PCR] and/or antibody test) presenting as severe COVID-19 requiring hospitalization;
3. Severe pneumonia, acute lung injury, or ARDS confirmed by computed tomography (CT) or X-ray at Screening or within the 3 days prior to Screening, as part of the patient's routine clinical care;
4. Respiratory distress requiring mechanical ventilation, which can be either invasive (requiring endotracheal intubation) or noninvasive (with continuous positive airway pressure [CPAP] or bilevel positive airway pressure [BiPAP]);
5. Body weight≥40 kg at the time of providing informed consent;
6. Male or female; and
7. Female patients of childbearing potential and male patients with female partners of childbearing potential must follow protocol-specified contraception guidance for avoiding pregnancy for 8 months after treatment with the study drug.

Patients are excluded from the study if any of the following criteria apply:
1. Patient is not expected to survive for more than 24 hours.
1. Patient is on invasive mechanical ventilation with intubation for more than 48 hours prior to Screening;
2. Severe pre-existing cardiac disease (i.e., New York Heart Association Class 3 or Class 4, acute coronary syndrome, or persistent ventricular tachyarrhythmias);
3. Patient has an unresolved Neisseria meningitidis infection;
4. Use of the following medications and therapies: (a) current treatment with a complement inhibitor or (b) intravenous immunoglobulin (IVIg) within 4 weeks prior to randomization on Day 1;
5. Treatment with investigational therapy in a clinical study within 30 days before randomization, or within 5 half-lives of that investigational therapy, whichever is greater. Exceptions: (a) investigational therapies are allowed if received as part of best supportive care through an expanded access protocol or emergency approval for the treatment of COVID 19 and (b) Investigational antiviral therapies (such as remdesivir) are allowed even if received as part of a clinical study;
6. Female patients who are breastfeeding or who have a positive pregnancy test result at Screening;
7. History of hypersensitivity to any ingredient contained in the study drug, including hypersensitivity to murine proteins; or
8. Patient who is not currently vaccinated against N. meningitidis, unless the patient agrees to receive prophylactic treatment with appropriate antibiotics for at least 8 months after the last infusion of study drug or until at least 2 weeks after the patient receives vaccination against N. meningitidis.

6. Study Drug

Ravulizumab, a recombinant humanized anti-C5 mAb composed of two 448 amino acid heavy chains and two 214 amino acid light chains, is an IgG2/4 kappa immunoglobulin consisting of human constant regions, and murine complementarity-determining regions grafted onto human framework light- and heavy-chain variable regions. Ravulizumab is produced in Chinese hamster ovarian cell lines and was designed through minimal targeted engineering of eculizumab by introducing 4 unique amino acid substitutions to its heavy chain to extend antibody half-life.

Ravulizumab drug product is supplied for clinical studies as a sterile, preservative-free 10 mg/mL solution in single-use vials and designed for infusion by diluting into commercially available saline (0.9% sodium chloride injection; country-specific pharmacopeia) for administration via IV infusion.

The proposed dosage regimen for the treatment of patients with COVID-19 who are ≥18 years and ≥40 kg and are randomized to ravulizumab plus BSC is presented in Table 26.

TABLE 26
Ravulizumab Dosage Regimen for COVID-19 Severe Pneumonia,
Acute Lung Injury, or Acute Respiratory Distress Syndrome

| Patient Body | Day 1 | Day 5 | Day 10 | Day 15 |
|---|---|---|---|---|
| Weight (kg)¹ | Dose (mg) | Dose (mg) | Dose (mg) | Dose (mg) |
| 40 to <60 | 2400 | 600 | 600 | 900 |
| 60 to <100 | 2700 | 900 | 900 | 900 |
| ≥100 | 3000 | 900 | 900 | 900 |

¹The patient's body weight will be recorded on the day of the infusion visit. If the weight at the day of the infusion cannot be obtained, the weight recorded during the previous study visit may be used.

Ravulizumab drug product is formulated at pH 7.0 and each 30 mL vial contains 300 mg of ravulizumab, 0.02% polysorbate 80, 150 mM sodium chloride, 6.63 mM sodium phosphate dibasic, 3.34 mM sodium phosphate monobasic, and Water for Injection, United States Pharmacopeia.

The ravulizumab admixture is administered to the patient using an IV tubing set via an infusion pump followed by an IV flush. Use of a 0.2 micron filter is required during the infusion. The IV flush is infused at the same rate of the infusion and end of flush is considered the end of infusion. The IV flush volume is not to be included in the total volume of study drug administered. Additional details are provided in the Pharmacy Manual.

Ravulizumab is manufactured and supplied in single 30 mL vials as a solution concentration of 10 mg/mL (Table 17). Each vial contains 300 mg of ravulizumab for IV administration.

Stability studies of the diluted admixture of ravulizumab (10 mg/mL) in 0.9% sodium chloride injection support an in-use stability of 6 hours at room temperature at 23° C.-27° C. (73° F.-80° F.) and 24 hours when refrigerated at 2° C.-8° C. (36° F.-46° F.).

Ravulizumab vials are not frozen or shaken.

7. Concomitant Therapy

Patients may receive appropriate concomitant medications, including antivirals, as part of BSC during this clinical study, unless prohibited per exclusion criteria.

Concomitant medications considered relevant to treatment of COVID-19 or ravulizumab treatment (e.g., vaccines, antimicrobials, antimalarials, antivirals, steroids, and vasopressors) that the patient is receiving at the time of enrollment or receives during the study must be recorded in the CRF/eCRF along with: (a) reason for use, (b) rates of administration, including start and end dates, and (c) dosage information including dose and frequency.

Use of the following medications and therapies is prohibited for the specified duration prior to Screening and for the duration of the study: (a) current treatment with a complement inhibitor, and (b) intravenous immunoglobulin (IVIg) within 4 weeks prior to randomization on Day 1.

8. Vaccination or Antibiotic Prophylaxis

Confirmation of meningococcal vaccination within the past 5 years prior to dosing for patients randomized to ravulizumab plus BSC. If vaccination cannot be confirmed, the patient should receive prophylactic antibiotics prior to initiating ravulizumab treatment and for at least 8 months from the last infusion of ravulizumab. When patients are vaccinated less than 2 weeks prior to treatment with ravulizumab or after initiation of ravulizumab, they should continue antibiotic prophylaxis for at least 2 weeks after meningococcal vaccination.

9. Screening Assessments

The SARS-CoV-2 infection is evaluated at the designated hospital. A confirmed positive result (e.g., via PCR and/or antibody test) is required before randomization.

Chest CT or X-ray scans are performed during the Screening Period to confirm findings consistent with severe pneumonia, acute lung injury, or ARDS in patients with COVID-19. Scans performed during the course of the patient's clinical care are accepted and expected to fulfil this diagnostic inclusion criterion for Study ALXN1210-COV-305.

Urine or serum pregnancy tests (beta human chorionic gonadotropin) are performed in all female patients. A negative pregnancy test result is required before administration of ravulizumab.

10. Efficacy Assessments

Survival at Day 29 is determined.

The following secondary efficacy parameters are also measured through Day 29: (a) mechanical ventilation status, (b) time in the intensive care unit (ICU), (c) sequential Organ Failure Assessment (SOFA) score, (d) oxygen saturation levels (peripheral capillary oxygen saturation [SpO2]), (e) supplemental oxygen status (fraction of inspired oxygen [FiO2]), and (f) duration of hospitalization.

The following secondary efficacy parameter is measured at Day 60 and Day 90: survival (based on all-cause mortality).

11. Sequential Organ Failure Assessment Score

Multiple organ failure is a significant indicator of mortality in patients admitted to the ICU. In this study, patients are evaluated using the SOFA score, an assessment tool that includes a review of 6 organ systems: respiratory, renal, hepatic, cardiac, coagulation, and central nervous system (Vincent, 1998; see Table 18). Each organ system is scored from 0 to 4 points using the worst value observed within the previous 24 hours (Table 18).

Arterial blood gas may not be drawn on a protocol-specified visit day; therefore, the assessment of partial pressure of oxygen (PaO2) is optional and the highly correlated SpO2 will be a surrogate for the respiratory system assessment.

12. Physical Examinations

The following safety-related parameters will be measured through Day 29. Complete or abbreviated physical examinations are assessed by the Investigator or designee. A complete physical examination includes, at a minimum, assessments of the skin, head, ears, eyes, nose, throat, neck, lymph nodes, chest, heart, abdomen, extremities, and musculoskeletal. An abbreviated physical examination includes at a minimum, assessment of the respiratory system and cardiovascular systems. Body weight is measured, but if the site does not have the capacity to measure the patient's body weight it should be estimated using best judgement.

Vital sign measurements include systolic and diastolic blood pressure (millimeters of mercury [mm Hg]), heart rate (HR, beats/minute), respiratory rate (RR, breaths/minute), and temperature (degrees Celsius [° C.] or degrees Fahrenheit [° F.]). Vital sign measurements are taken predose on dosing days.

A single 12 lead electrocardiogram (ECG) is conducted to obtain HR, pulse rate (PR) interval, combination of the Q wave, R wave and S wave (QRS) interval, interval between the start of the Q wave and the end of the T wave (QT), and the corrected QT (QTc) interval(s).

13. Glasgow Coma Scale

The Glasgow Coma Scale (GCS) is a validated prognostic tool used in the clinical assessment of unconsciousness (e.g, patients who are comatose) (Sternbach, 2000). The GCS is comprised of 3 domains—eye response, verbal response, and motor response and within each domain contains a subset of responses that are separately assigned a score (see Table 19). The GCS has also been used in the critical care setting as an aid in managing respiratory support. A total GCS score of <8 is indicative of a patient's need for endotracheal intubation. The GCS is measured to enable calculation of the secondary efficacy endpoint, SOFA score.

14. Vaccine and Antibiotic Prophylaxis

It is anticipated that patients randomized to ravulizumab plus BSC who have not received a meningococcal vaccination within the past 5 years may be unable to receive meningococcal vaccinations prior to initiating treatment with ravulizumab during this study. If vaccination cannot be confirmed, the patient receive prophylactic antibiotics against meningococcal infection prior to initiating ravulizumab treatment and for at least 8 months from the last infusion of ravulizumab.

When patients can be vaccinated, vaccines against meningococcal serotypes A, C, Y, W135, and B, where available, are recommended to prevent common pathogenic meningococcal serotypes. Patients must be vaccinated or revaccinated according to the current national vaccination guidelines or local practice for vaccination use with complement inhibitors (e.g., ravulizumab). Vaccination may not be sufficient to prevent meningococcal infection. Consideration should be given per official guidance and local practice on the appropriate use of antibacterial agents. When patients are vaccinated after initiation of ravulizumab, they continue antibiotic prophylaxis for at least 2 weeks after meningococcal vaccination.

15. Clinical Improvement at Day 29

A reduction in the time to clinical improvement, especially when the patient is treated within a short timeframe from symptom onset has been reported in studies comparing antivirals to placebo (Wang, 2020). Time to clinical improvement is evaluated during this study and is defined as a live discharge, a decrease from of least 2 points (i.e., #5 to #3) from baseline, or both. The modified 6-category ordinal scale (set forth in Table 27) is used to evaluated clinical improvement.

TABLE 27
Modified 6-Category Ordinal Scale

| | |
|---|---|
| 1 | Discharged |
| 2 | Hospitalized, not requiring supplemental oxygen |
| 3 | Hospitalized, requiring supplemental oxygen |
| 4 | Hospitalized, requiring noninvasive mechanical ventilation |
| 5 | Hospitalized, requiring invasive mechanical ventilation |
| 6 | Death |

16. 12-Item Short Form at Days 29, 60, and 90

The Short-Form (SF)-12 is a validated health-related quality of life (HR-QoL) instrument that is widely used across a broad spectrum of disease indications. Adapted from the 36-item SF survey that was designed to evaluate physical and mental health status, the SF-12 survey contains only 12 questions but covers the same 8 domains. There is a further stratification into 2 summary measures (Physical Component Summary [PCS-12] and Mental Component Summary [MCS-12]) as specified below in Table 27.

The PCS-12 and MCS-12 summary measures are scored using a norm-based method (i.e., mean=50, SD=10) (Jenkinson, 1997). A PCS-12 or MCS-12 score of 50 indicates an average score with respect to a healthy population. Scores lower than 50 reflect less than average health and scores greater than 50 reflect better than average health (Ware, 1995).

The SF-12 assumes a recall of 1 week before responding to questions. The survey is anticipated to be completed in several minutes and can be completed by the patient or via an interviewer (in-person or over the telephone).

17. EuroQol-5 Dimension-5 Level at Days 29, 60, and 90

The EuroQol 5-dimension, 5 severity level (EQ-5D-5L) questionnaire is a brief, validated, HR-QoL instrument that is intended to assess the patient's health status at the time of administration. The questionnaire contains 5 dimensions (mobility, self-care, usual activities, pain/discomfort, and anxiety/depression), each of which includes 5 response variables (no problems, slight problems, moderate problems, severe problems, and extreme problems) (EQ—5D, 2019). There is no summary score generated upon completion, but rather a 5-digit profile (termed “health state”) based on each of the dimensions that can be further converted to a single numerical score (index value). Value sets (a collection of index values) have been derived for multiple countries/regions.

A vertical visual analogue scale (VAS) is included for patients to indicate a self-rated estimate of their health. The VAS ranges from 100 (best health you can imagine) to 0 (worst health you can imagine).

The EQ-5D-5L questionnaire and VAS are anticipated to be completed in several minutes and can be completed by the patient, via an interviewer (in-person or over the telephone); or via proxy.

18. Adverse Events and Serious Adverse Events

The definitions of AEs and SAEs are set forth in Tables 20 and 21.

All AEs are reported to the Investigator or qualified designee by the patient (or, when appropriate, by a caregiver, surrogate, or the patient's legally acceptable representative).

The Investigator and any qualified designees are responsible for detecting, documenting, and recording events that meet the definition of an AE or SAE and remain responsible for following up AEs that are serious, considered related to the study intervention or study procedures, or that caused the patient to discontinue the study intervention.

All AEs and SAEs are collected from the time of informed consent until through the timepoints specified in the Schedule of Assessments.

19. Pharmacokinetics, Pharmacodynamics, and Biomarkers

Samples are collected from patients randomized to ravulizumab plus BSC as specified in the Schedule of Activities to determine serum concentrations of ravulizumab. The actual date and time (24-hour clock time) of each sample is recorded.

Samples are collected from all patients as specified in the Schedule of Activities to assess the effect of ravulizumab on total and free C5 (for patients randomized to ravulizumab plus BSC) and determine complement activation in patients randomized to BSC alone. The actual date and time (24-hour clock time) of each sample is recorded.

Serum and plasma samples are collected from all patients for biomarker analysis to evaluate complement activation and related pathways and cardiovascular health, and their clinical response to ravulizumab. These biomarkers include complement pathway proteins (e.g., total and free C5, soluble C5b-9 [sC5b-9]), cytokines associated with inflammation and disease (eg, IL-1, IL-2R, IL-6, IL-8, IL-21, tumor necrosis factor [TNF]-b, Pentraxin-3, Citrullinated histone H3, and monocyte chemoattractant protein [MCP]-1), Factor II, and markers associated with cardiovascular disease (procalcitonin, myoglobin, high sensitivity troponin I [hs-TnI] and N-terminal pro-b-type natriuretic peptide [NT-proBNP]).

20. Immunogenicity

Antibodies to ALXN1210 (i.e., antidrug antibody [ADA]) are evaluated in serum samples collected from patients randomized to ravulizumab plus BSC according to the Schedule of Activities. Additionally, serum samples are also collected at the final visit from patients who discontinued ravulizumab or were withdrawn from the study.

Serum samples are screened for antibodies binding to ravulizumab and the titer of confirmed positive samples is reported. Other analyses can be performed to further characterize the immunogenicity of ravulizumab.

The detection and characterization of antibodies to ravulizumab is performed using a validated assay method. Samples collected for detection of antibodies to ravulizumab are also evaluated for study intervention serum concentration to enable interpretation of the antibody data. Confirmed antibody positive samples are further evaluated for antibody titer and the presence of neutralizing antibodies.

21. Statistical Considerations

The primary null hypothesis is that there is no difference in survival between ravulizumab plus BSC and BSC alone as measured by the difference in the proportions surviving at Day 29 between the 2 treatment groups. The alternative hypothesis is that ravulizumab plus BSC improves survival at Day 29 compared with BSC alone.

The null hypotheses associated with the secondary objectives are that ravulizumab plus BSC is no different than BSC alone for the respective endpoints; the alternative hypotheses are described below:
1. Number of days free of mechanical ventilation: The alternative hypothesis is that treatment with ravulizumab plus BSC increases the number days free of mechanical ventilation at Day 29 compared with BSC alone.
2. Duration of ICU stay: The alternative hypothesis is that treatment with ravulizumab plus BSC reduces the number days in the ICU at Day 29 compared with BSC alone.
3. Change in SOFA score: The alternative hypothesis is that treatment with ravulizumab plus BSC improves changes in SOFA score at Day 29 compared with BSC alone.
4. Change in SpO2/FiO2: The alternative hypothesis is that treatment with ravulizumab plus BSC improves changes in SpO2/FiO2 at Day 29 compared with BSC alone.
5. Duration of hospitalization: The alternative hypothesis is that treatment with ravulizumab plus BSC reduces the number days in the hospital at Day 29 compared with BSC alone.
6. Survival (based on all cause mortality) at Day 60 and Day 90: The alternative hypothesis is that ravulizumab plus BSC improves survival at Day 60 and Day 90 compared with BSC alone.

A sample size of 243 patients (162 ravulizumab+BSC, 81 BSC alone) is required to ensure at least 90% power and detect an improvement in survival from 60% in the BSC alone group to 80% in the ravulizumab+BSC group at Day 29. This sample size calculation assumes: (a) 1-sided Z-test of the difference in 2 proportions, (b) Type I error=0.025, (c) pooled variance, (d) 2:1 randomization on the 2 treatment groups, and (e) One interim analysis at 50% information which is after collecting primary efficacy data on approximately 122 patients. The early stopping boundaries for efficacy and futility (nonbinding) is constructed using a spending function as Lan DeMets spending function with O'Brien Fleming flavor and p spending function as Gamma(4) (Lan, 1983; Hwang, 1990).

Considering a nonevaluable rate of 10%, this study is planned to randomize approximately 270 patients (180 ravulizumab+BSC, 90 BSC alone).

The population sets used for analysis sets are set forth in Table 28.

The primary analysis is conducted when all patients have completed the Primary Evaluation Period. This analysis includes all efficacy, safety, and available PK/PD/immunogenicity study data for regulatory submission purposes and is the final analysis of the Primary Evaluation Period.

Summary statistics are presented by treatment group and by visit, where applicable. Descriptive statistics for continuous variables minimally include the number of patients, mean, standard deviation, median, minimum, and maximum. For categorical variables, frequencies and percentages are presented. Graphical displays are provided as appropriate. All statistical analyses are performed based on a 2-sided Type I error of 5%, unless otherwise noted.

Baseline is defined as the last available assessment on or before Day 1 for all patients. Day 1 is defined as the date of the first infusion of ravulizumab for patients randomized and dosed with ravulizumab and as the date of randomization for patients randomized but not dosed with ravulizumab.

Analyses are performed using SAS® software Version 9.4 or higher.

The primary efficacy endpoint is survival (based on all-cause mortality) at Day 29 and will be compared between the 2 treatment groups using a 1-sided Mantel-Haenszel (MH) test of the difference in 2 proportions stratified by intubated or not intubated on Day 1 and a Type I error of 0.025. The estimated MH risk difference is summarized along with the 95% confidence interval using Mantel-Haenszel stratum weights (Mantel, 1959) and the Sato variance estimator (Sato, 1989). Missing survival data for the primary analysis is imputed using a multiple imputation approach assuming the data are missing at random (MAR) using a logistic regression model with covariates for treatment group, the randomization stratification factor, age, sex, and presence of a pre-existing condition at baseline. Sensitivity analyses include the worst-case, all available, and best-case scenarios.

Survival is also analyzed using the method of Kaplan and Meier (KM) and compared using a log-rank test stratified by intubated or not intubated on Day 1 as a sensitivity analysis. Hazard ratio and risk reduction are summarized from a Cox proportional hazards model stratified by intubated or not intubated on Day 1. Confidence intervals (95%) are presented for the survival estimate at Day 29 based on the complementary log-log transformation. Kaplan-Meier curves for both treatment groups are produced.

A sensitivity analysis of the primary endpoint is also be performed using a 3-level categorical outcome of 3) alive and discharged from the ICU; 2) alive and in the ICU or 1) death. The 2 treatment groups are compared using an ordinal logistic regression with covariates for treatment group and the randomization stratification factor.

Additional sensitivity analyses include statistical models adjusting for age, randomization stratification factor, and other important baseline covariates. Subgroup analyses are also performed by age group, randomization stratification factor, and other important baseline covariates. The Statistical Analysis Plan (SAP) describes the sensitivity and subgroup analyses in greater detail.

Number of days free of mechanical ventilation at Day 29 is compared between treatment groups using an analysis of covariance (ANCOVA), adjusting for age, and randomization stratification factor, among survivors. Missing data is imputed using a multiple imputation approach assuming the data are MAR. Sensitivity analyses include the worst-case, all available, and best-case scenarios.

Duration of ICU stay at Day 29 are compared between treatment groups using an ANCOVA, adjusting for age and randomization stratification factor, among survivors. Missing data are imputed using a multiple imputation approach assuming the data are MAR. Sensitivity analyses include the worst-case, all available, and best-case scenarios.

Changes in SOFA score from Day 1 to Day 29 are summarized by treatment group and study visit for all patients and are analyzed using a mixed model for repeated measures (MMRM) with baseline SOFA score, age, randomization stratification factor, treatment group indicator, study day (Days 5, 10, 15, 22, and 29), and study day by treatment group interaction as covariates. Sensitivity analyses include imputations for missing data.

Change from baseline in SpO2/FiO2 at Day 29 are analyzed using a MMRM with baseline SpO2/FiO2, age, randomization stratification factor, treatment group indicator, study day (Days 5, 10, 15, 22, and 29), and study day by treatment group interaction as covariates. All patients are included in the model. Sensitivity analyses include imputations for missing data. Change from baseline in PaO2/FiO2 at Day 29 are also be analyzed using a MMRM with baseline PaO2/FiO2, age, randomization stratification factor, treatment group indicator, study day, and study day by treatment group interaction as fixed covariates. All patients are included in the model. Sensitivity analyses include imputations for missing data.

Duration of hospitalization at Day 29 are analyzed in a similar manner as duration of ICU stay.

Survival (based on all-cause mortality) at Day 60 and Day 90 is estimated using the KM method and compared using a log-rank test stratified by intubated or not intubated on Day 1. Hazard ratio and risk reduction are summarized from a Cox proportional hazards model stratified by intubated or not intubated on Day 1. Confidence intervals (95%) are presented for the survival estimates at Day 60 and Day 90 based on the complementary loglog transformation. Kaplan and Meier curves for both treatment groups are produced.

A closed testing procedure is applied to control the type I error for the analyses of the primary and secondary endpoints. If the primary endpoint is statistically significant in favor of ravulizumab, the secondary endpoints are evaluated according to the following rank order:
1. Number of days free of mechanical ventilation at Day 29,
2. Duration of ICU stay at Day 29,
3. Change from baseline in SOFA score at Day 29,
4. Change from baseline in SpO2/FiO2 at Day 29,
5. Duration of hospitalization at Day 29.

The hypothesis testing proceeds from highest rank (#1) the number of days free of mechanical ventilation at Day 29 to the lowest rank (#5) duration of hospitalization at Day 29, and if statistical significance is not achieved at an endpoint (p≥0.05), then endpoints of lower rank are not considered to be statistically significant. Confidence intervals and p-values are presented for all secondary efficacy endpoints for descriptive purposes, regardless of the outcome of the closed testing procedure.

An additional secondary endpoint is assessed beyond Day 29 regardless of the results of the closed testing procedure: Survival (based on all-cause mortality) at Day 60 and Day 90.

All safety analyses are made on the Safety Set (SS). Safety results are reported by treatment group.

The analysis and reporting of AEs and SAEs is based on TEAEs and TESAEs, defined as AEs and SAEs with onset during or after treatment with ravulizumab. The incidence of TEAEs and TESAEs is summarized by System Organ Class and Preferred Term, with additional summaries showing relationship to ravulizumab, severity, TEAEs or TESAEs leading to ravulizumab discontinuation, and TESAEs resulting in death.

Laboratory measurements, as well as their changes from baseline at each visit and shift from baseline, if applicable, are summarized. Vital sign measurements, physical examination findings, and ECG data are also summarized over time.

All patients who have evaluable PK/PD data are used to summarize PK/PD parameters for ravulizumab. Descriptive statistics of ravulizumab concentration data are presented for patients randomized and treated with ravulizumab for each scheduled sampling timepoint.

Total and free C5 concentrations are evaluated by assessing the absolute values and changes and percentage changes from baseline, as appropriate. Descriptive statistics are presented by treatment group and for each scheduled sampling timepoint.

Serum and plasma biomarkers' actual values, and changes from baseline, and their association with observed clinical responses to ravulizumab are summarized over time, as appropriate. Biomarker data is only summarized at the final analysis at the end of the study.

Blood samples are collected for biomarker analyses and the data may be used for future exploratory research related to complement activation and inflammatory processes. The samples may also be used to develop tests/assays including diagnostic tests related to C5 inhibitors and COVID 19 with clinical presentation of severe pneumonia, acute lung injury, or ARDS.

The incidence and titers for ADAs to ravulizumab are summarized in tabular format by treatment group. The proportion of patients ever positive and the proportion of patients always negative may be explored. Confirmed ADA positive samples are evaluated for the presence of neutralizing antibodies.

Incidence of and time to progression to renal failure requiring dialysis at Day 29 are analyzed in a similar manner as the primary endpoint.

Time to clinical improvement is analyzed using the KM method and compared using a log-rank test stratified by intubated or not intubated on Day 1.

The SF-12 PCS and MCS scores and EQ-5D-5L index and VAS scores are analyzed using an ANCOVA, adjusting for age and the randomization stratification factor.

An interim analysis for efficacy and futility is conducted when approximately 122 patients have completed Day 29. If the stopping criteria are met, the study may be terminated early for efficacy or futility depending on which stopping boundary is crossed. The early stopping boundaries for efficacy and futility (nonbinding) are constructed using α-spending function as Lan-DeMets (O'Brien-Fleming) spending function and β-spending function as Gamma (-4). A 1-sided t-test based on the results from combining all imputed datasets for overall inference is used with an overall Type I error of 0.025.

Provided the study was not stopped early for efficacy or futility, the final primary analysis is conducted when all patients have completed the Primary Evaluation Period. This analysis includes all efficacy, safety, and available PK/PD/immunogenicity study data for regulatory submission purposes. This analysis is not considered an interim analysis.

Protocol-required laboratory assessments are set forth in Table 28.

TABLE 28
Protocol-required Laboratory Assessments
The following parameters may be analyzed by the local laboratory:

| | |
|---|---|
| Hematology Panel | Clinical Chemistry Panel |
| Haptoglobin | Alanine aminotransferase |
| Hematocrit | Aspartate aminotransferase |
| Hemoglobin | Blood urea nitrogen |
| Platelet count | C-reactive protein |
| WBC count | Creatinine |
| WBC differential | Lactate dehydrogenase |
| Coagulation Panel | Sodium |
| D-dimer | Total bilirubin (direct and indirect) |
| International normalized ratio | Ferritin |
| Partial thromboplastin time | Other |
| Prothrombin time | Arterial blood gas (when available) |
| Fibrinogen | Beta human chorionic gonadotropin (all females |
| Urinalysis | of childbearing potential only; optional if urine |
| Albumin | pregnancy test is negative) |
| Creatinine | Direct Coombs test |
| Protein | |

The following parameters may be analyzed by the central laboratory or specialty laboratories:

| | |
|---|---|
| Inflammatory Biomarkers | Other Biomarkers |
| IL1, IL-2R, IL-6, IL-8, IL-21, Pentraxin-3, and | monocyte chemoattractant protein-1 (MCP-1) |
| Citrullinated histone H3 | TNF-b |
| Complement Pathway Biomarkers | Immunogenicity assay |
| Total and free C5 and sC5b-9 | (only collect from patients randomized to |
| Cardiac Biomarkers | ravulizumab + BSC) |
| Procalcitonin | Pharmacokinetic assay |
| Myoglobin | (only collect from patients randomized to |
| NT-proBNP (N-terminal pro b-type natriuretic | ravulizumab + BSC) |
| peptide) | Coagulation Biomarker |
| high sensitivity troponin I (hs-TnI) | Factor II |

### Example 10: Eculizumab as an Emergency Treatment for Adult Patients with Severe COVID-19 in the Intensive Care Unit

Since December 2019, a novel severe acute respiratory syndrome coronavirus (SARS-CoV-2) has spread from Wuhan, China, and as of May 26, 2020, has infected approximately 5,559,000 people in 188 countries, causing >349,000 deaths (see, e.g., Johns Hopkins University (2020) COVID-19 Dashboard by the Center for Systems Science and Engineering (CSSE) at Johns Hopkins University. Available at world wide web coronavirus.jhu.edu/map.html. Accessed May 21, 2020; and Guan et al. (2020), N Engl J Med 382:1708-1720).

The crude hospitalization rate for patients with COVID-19, the illness caused by SARS-CoV-2, is approximately 67.9 per 100,000 persons in the United States and 150 per 100,000 persons in France (see, e.g., US Centers for Disease Control and Prevention COVIDView: A weekly surveillance summary of U.S. COVID-19 Activity, key updates for week 16, ending Apr. 18, 2020. Available at world wide web cdc.gov/coronavirus/2019-ncov/covid-data/covidview/index.html. Accessed May 21, 2020; and Santé Publique France Infection au nouveau Coronavirus (SARS-CoV-2), COVID-19, France et Monde. Available at world wide web santepubliquefrance.fr/maladies-et-traumatismes/maladies-et-infections-respiratoires/infection-a-coronavirus/articles/infection-au-nouveau-coronavirus-sars-cov-2-covid-19-france-et-monde. Accessed May 21). According to published reports, 5-32% of confirmed, hospitalized patients require admission to the intensive care unit (ICU) (see, e.g., Guan et al. (2020); and Huang et al. (2020), Lancet 395:497-506). In these severe COVID-19 cases, clinical manifestations may include pneumonia; acute respiratory distress syndrome (ARDS), necessitating respiratory support; acute kidney, cardiac, and liver injury; sepsis; and disseminated intravascular coagulopathy (see, e.g., Guan et al. (2020); and Huang et al. (2020)).

Efforts to understand the biological mechanisms underlying acute lung injury have elucidated a critical role for the complement system, an important component of innate and adaptive immunity (see, e.g., Pandya et al. (2014), Am J Respir Cell Mol Biol 51:467-473). Complement signaling orchestrates key immuno-protective and anti-inflammatory functions, enabling clearance of pathogens and apoptotic cells (see, e.g., Pandya et al. (2014). However, complement activation and subsequent production of the proinflammatory anaphylatoxin C5a, a cleavage product of terminal complement protein C5, and formation of the terminal complement complex C5b-9, precipitate biological sequelae that can be harmful if unchecked, including activation of endothelial and phagocytic cells, generation of reactive oxygen species, and initiation of an inflammatory cytokine storm (see, e.g., Wang et al. (2015), Emerg Microbes Infect 4:e28). C5a-mediated effects have been shown to play a critical role in the development of acute lung injury induced by highly pathogenic viruses (see, e.g., Wang et al. (2015), Emerg Microbes Infect 4:e28). In mice, complement inhibition directed at C5a or upstream proteins (i.e, C3, C3a) reduced lung injury after SARS-CoV (see, e.g., Gralinski et al. (2018) mBio 9:e01753-01718) and influenza H5N1 virus infection (see, e.g., Sun et al. (2013), Am J Respir Cell Mol Biol 49:221-230). In SARS-CoV-infected mice, this occurred without change in viral titer (see, e.g., Gralinski et al. (2018)), suggesting that complement inhibition may provide protection from lung injury independent of viral load. Similar protection after C5a inhibition has been observed in animal models of Middle East respiratory syndrome coronavirus (see, e.g., Jiang et al. (2018), Emerg Microbes Infect 7:77), avian influenza H5N1 virus (see, e.g., Sun et al. (2013)), and H7N9 virus infection (see, e.g., Sun et al. (2015), Clin Infect Dis 60:586-595) Clinical studies have provided evidence for excessive complement activation in patients with SARS (see, e.g., Pang et al. (2006), Clin Chem 52:421-429) and H1N1 influenza (see, e.g., Ohta et al. (2011), Microbiol Immunol 55:191-198; and Berdal et al. (2011), J Infect 63:308-316) correlating to some degree with disease severity (see, e.g., Pang et al. (2006); and Berdal et al. (2011), J Infect 63:308-316). Other work has shown that progression of SARS illness is accompanied by the development of autoantibodies that mediate a form of complement-dependent cytotoxicity that may lead to further lung injury. Collectively, these observations suggest that blocking complement activation using a C5 inhibitor may be an effective treatment option for SARS-CoV-mediated disease.

Eculizumab is a humanized monoclonal antibody that is approved for the treatment of patients with paroxysmal nocturnal hemoglobinuria (PNH), atypical hemolytic uremic syndrome (aHUS), generalized myasthenia gravis (gMG), and neuromyelitis optica spectrum disorder (NMOSD) (see, e.g., Hillmen et al. (2006), N Engl J Med 355:1233-1243; Legendre et al. (2013), N Engl J Med 368:2169-2181; Pittock et al. (2019), N Engl J Med 381:614-625; Howard et al. (2017), Lancet Neurol 16:976-986; and SOLIRIS® (eculizumab). Summary of Product Characteristics, Alexion Europe SAS, Levallois-Perret, France, 2019). Eculizumab binds to terminal complement C5 with high affinity, inhibiting its cleavage to C5a and C5b and preventing the formation of C5b-9, which has variable effects, including lytic, proinflammatory, and prothrombotic properties (see, e.g., Pandya et al. (2014), Am J Respir Cell Mol Biol 51:467-473; Merle et al. (2015), Front Immunol 6:257; and Morgan et al. (2016), Immunol Rev 274:141-15). Selective C5 blockade preserves upstream complement component activity essential for opsonization of microorganisms and prevention of immune complex disorders (see, e.g., Merle et al. (2015), Front Immunol 6:257; and Matis et al. (1995) Complement-specific antibodies: designing novel anti-inflammatories. Nat Med 1:839-842). The ability of eculizumab to prevent tissue injury and the proinflammatory and prothrombotic effects of C5a and C5b-9 while preserving upstream immunoprotective and immunoregulatory functions suggests it may be an effective therapeutic for severe respiratory illness, including severe COVID-19. A recent case report and small case series further suggest this could be a promising therapeutic approach (see, e.g., Diurno et al. (2020), Eur Rev Med Pharmacol Sci 24:4040-4047; and Pitts T C (2020) A preliminary update to the Soliris to Stop Immune Mediated Death in Covid-19 (SOLID-C19) compassionate use study. Hudson Medical. Available at world wide web hudsonmedical.com/articles/soliris-stop-death-covid-19/. Accessed May 21). Disclosed herein are results from a first proof-of-concept study of eculizumab as experimental emergency treatment for patients with severe COVID-19 who were admitted to the ICU.

1. Methods

This controlled before-after study included a consecutive cohort of patients≥18 years of age admitted to the ICU between Mar. 10 and May 5, 2020, with severe COVID-19 confirmed by reverse-transcriptase polymerase chain reaction; symptomatic bilateral pulmonary infiltrates confirmed by computed tomography or chest X-ray ≤7 days before screening; and severe pneumonia, acute lung injury, or ARDS requiring supplemental oxygen. Patients were treated according to institutional and governmental guidelines for severe COVID-19, which included respiratory management, anticoagulants, antivirals, and antibiotics when indicated. On Mar. 19, 2020, eculizumab (300-mg/30-mL vials for intravenous infusion) were provided under the expanded access program (EAP) as experimental emergency treatment for adults with COVID-19 and severe pneumonia, acute lung injury, or ARDS, subsequent to physician request and in accordance with relevant national regulatory authorities. Patients were not eligible for this treatment if they were <40 kg; required <6 L/min of oxygen to maintain arterial oxygen saturation>90%; or had life expectancy≤24 hours, unresolved Neisseria meningitidis infection, or hypersensitivity to murine proteins or to an excipient of eculizumab. Patients were not selected for treatment; rather, treatments were assigned consecutively based on availability of eculizumab at the time of ICU admission, without randomization. “Before” periods included times in which eculizumab treatment was not available (without eculizumab), and “after” periods included times in which eculizumab was provided.

Upon initial receipt of eculizumab, 10 consecutive patients received emergency treatment according to dosing procedures within a subsequently approved EAP protocol. Subsequently, 25 patients were formally enrolled into the approved EAP protocol. Single infusions of eculizumab 900 mg were administered intravenously over 45 minutes on days 1 (within 7 days of confirmed pneumonia or ARDS), 8, 15, and 22. This regimen was designed to target immediate, complete, and sustained terminal complement inhibition and was based on the approved induction dosage regimen for aHUS, gMG, and NMOSD (see, e.g., Jiang et al. (2018), Emerg Microbes Infect 7:77). Patients received vaccination and prophylactic antibiotics against meningococcal infection (e.g., cefotaxime) before initiating eculizumab and for ≥60 days after the last infusion. Patients released from the ICU were required to remain hospitalized under quarantine until they were symptom-free for ≥2 days.

2. Study Assessments and Outcomes

Baseline patient demographic, clinical characteristics, and concomitant medication use were recorded in the hospital electronic health records at ICU admission; physical examination, vital signs, and laboratory tests were recorded at ICU admission and during treatment. Antiviral treatment, respiratory support, vasopressor therapy, and renal replacement therapy were also recorded. Serum samples for analysis of biomarkers of complement activation were collected before each infusion (see below).

Laboratory parameters were assayed at an ISO 15189-certified laboratory at the Hospital. Cytokine quantifications were performed on a Bio-Rad Bioplex 200 (Bio-Rad Laboratories, Inc., Marnes-la-Coquette, France) in duplicate using the Bio-Plex Pro Human Cytokine Screening Panel (Bio-Rad) according to the manufacturer's instructions on all consecutive biobanked samples in a blinded fashion. Values below and above the instrument's detection range were rounded to the nearest value in the detection range for the tested analyte.

Concomitant with clinical and biological data, CH50 activity; circulating levels of C3, C4, and soluble C5b-9; and free eculizumab levels were measured at days 1 and 7 after eculizumab infusion. For patients treated without eculizumab, day 1 was defined as the day of first complement assessment. The CH50 assay has been previously described in detail. Using this technique, blocked CH50 was defined as ≤20%, which reflects the presence of <5% of functional C5, and unblocked CH50 was defined as >20%. Soluble C5b-9 levels were determined using the MicroVue SC5b-9 Plus EIA kit (Quidel, San Diego, CA) according to the manufacturer's instructions. Normal values were determined with plasma from 68 healthy donors (<340 ng/mL). Circulating levels of C3 and C4 were also studied by nephelometry according to the instructions of the manufacturer (Siemens, Malvern, PA). Determination of free eculizumab concentration in plasma was performed using a homemade enzyme-linked immunosorbent assay, as previously reported by de Latour et al. (Blood 125:775-83, 2015).

The prespecified primary outcome was survival (based on all-cause mortality) at day 15, representative of approximate median time to death in previous reports. Additional outcomes of interest were survival at day 28, number of days alive and free of mechanical ventilation at day 15 in patients ventilated at baseline, number of ICU-free days, and change in oxygenation status at day 15. Other outcomes included changes over time in respiratory function, markers of tissue hypoxia, hematology and clinical chemistry parameters, inflammatory mediators, serum eculizumab, and soluble biomarkers associated with complement activation. Safety was characterized based on the incidence of treatment-emergent serious adverse events (TESAEs) of special interest (infections, hematologic disorders, associated with critical care).

The EAP protocol was approved by the local regulatory board and conducted in accordance with the Declaration of Helsinki, International Council for Harmonisation Good Clinical Practice guidelines, and local laws and regulations. Owing to the “state of health emergency,” deferred informed consent was recorded. The sponsor designed the EAP and provided eculizumab. Clinical and laboratory variables were independently extracted from hospital electronic health records. Assessments were recorded by research staff and analyzed independently. All authors had full and independent access to all data and vouch for the integrity, accuracy, and completeness of the data and analysis and to adherence to the EAP protocol.

3. Statistical Analysis

Because this was a proof-of-concept study, there was no formal sample size calculation; analyses included all patients with severe COVID-19 admitted to the ICU between Mar. 10 and May 5, 2020. The index date (baseline; day 0) was the date of ICU admission. Data were censored on May 22, 2020. Baseline demographics, clinical characteristics, laboratory values were compared using Fisher's exact test (categorical variables) or the Wilcoxon test (continuous variables). Survival was estimated using the Kaplan-Meier method. Kaplan-Meier survival curves were compared using a log-rank test. Hazard ratios (HRs) and associated 95% CIs were estimated using a Cox proportional-hazards model adjusted for sex and Simplified Acute Physiology Score (SAPS II) with exposure as a time-dependent variable. Actual proportions for survival and rates of TESAEs were compared using Fisher's exact test. Changes in laboratory values over time were assessed using linear mixed models for longitudinal data with a time by group effect. Changes in C5b-9 levels and days alive and free of mechanical ventilation were analyzed using the Wilcoxon test. P values were two-sided. Analyses were performed with R version 3.5.1 (R Foundation for Statistical Computing).

4. Results

Between Mar. 10 and May 5, 2020, 80 patients were admitted to the ICU, including 35 who received treatment with eculizumab and 45 who did not. Median (range) ICU follow-up was 20 (13-34) days with eculizumab and 10 (8-13) days without eculizumab; median (range) hospital follow-up was 39 (33-not reached) and 17 (14-21) days, respectively. Mean (SD) time from ICU admission to first dose of eculizumab was 3.5 (2.7) days. At time of data cutoff, 27 patients (77%) had received all 4 scheduled eculizumab doses, 1 (3%) received 3 doses, 5 (14%) received 2 doses, and 2 (6%) received 1 dose. One patient refused the fourth dose after being discharged from the ICU, and 2 patients were discharged home before the third infusion; 3 died before receiving the third infusion and 2 before the second. Patient baseline characteristics, including diagnosis of severe pneumonia and ARDS and markers of complement activation and infection, did not differ significantly between groups (Tables 29 and 30). Median age of patients treated with versus without eculizumab was 64 and 55 years, respectively; 63% versus 76% were men; and 46% versus 52% had severe ARDS (PaO2/FiO2≤100 mmHg). Median time from first symptoms to hospitalization was 6 days (both groups). Treatment use per institutional and French national guidelines was generally similar between groups receiving or not receiving eculizumab; the proportion of patients who received remdesivir was 3% and 16%, respectively, and who received lopinavir-ritonavir was 11% and 27%, respectively (Table 29).

TABLE 29
Patient Baseline^{a} Demographic and Clinical Characteristics

| | With | | Without | | |
|---|---|---|---|---|---|
| | Eculizumab | | Eculizumab | | P |
| Characteristic | (n = 35) | | (n = 45) | | Value^{b} |
| Age, y | | | | | |
| Mean (SD) | 62 | (14.3) | 59 | (17.9) | 0.43 |
| Median (IQR) | 64 | (54-71) | 55 | (43-73) | — |
| Men, n (%) | 22 | (63) | 34 | (76) | 0.31 |
| BMI, kg/m² | | | | | |
| Mean (SD) | 27.3 | (6.4)^{c} | 26.5 | (5.0) | — |
| Median (IQR) | 26.2 | (22.2-31.1)^{c} | 26.1 | (23.5-28.5) | 0.84 |
| Time from symptoms onset to | | | | | |
| hospitalization^{d, d} | | | | | |
| Mean (SD) | 6 | (5)^{c} | 8 | (6) | — |
| Median (IQR) | 6 | (3-8)^{c} | 6 | (5-9) | 0.19 |
| Time from hospitalization to ICU | | | | | |
| admission,^{d} | | | | | |
| Mean (SD) | 3 | (4)^{c} | 2 | (2) | — |
| Median (IQR) | 2 | (0-5)^{c} | 1 | (0-3) | 0.18 |
| SAPS II^{e} | | | | | |
| Mean (SD) | 59 | (20) | 52 | (20)^{f} | — |
| Median (IQR) | 66 | (48-71) | 56 | (46-67)^{f} | 0.08 |
| SOFA score^{g} | | | | | |
| Mean (SD) | 9 | (3) | 8 | (5)^{f} | — |
| Median (IQR) | 9 | (8-12) | 8 | (3-12)^{f} | 0.22 |
| PaO2/FiO2, mmHg, n (%)^{h} | | | | | 0.81 |
| ≤100 | 13 | (46) | 22 | (52) | — |
| >100 | 15 | (54) | 20 | (48) | — |
| Mechanical ventilation, n (%) | 21 | (60) | 31 | (69) | 0.48 |
| Renal replacement therapy, n (%) | 6 | (17) | 15 | (33) | 0.13 |
| Comorbidities, n (%) | | | | | |
| Hypertension | 14 | (40) | 16 | (36) | 0.82 |
| Coronary heart disease | 6 | (17) | 9 | (20) | 0.78 |
| Cancer | 6 | (17) | 7 | (16) | 1 |
| Kidney failure | 1 | (3) | 4 | (9) | 0.38 |
| COPD | 0 | | 3 | (7) | 0.25 |
| Medication use, n (%) | | | | | |
| Heparin, 4000 IU/d | 35 | (100) | 44 | (98) | 1 |
| Chloroquine | 30 | (86) | 41 | (91) | 0.49 |
| Vasopressors | 6 | (17) | 9 | (20) | 0.78 |
| Lopinavir-ritonavir | 4 | (11) | 12 | (27) | 0.16 |
| Corticosteroids | 5 | (14) | 4 | (9) | 0.49 |
| ACE inhibitor | 2 | (6) | 6 | (13) | 0.46 |
| Angiotensin II receptor blockers | 2 | (6) | 3 | (7) | 1 |
| Remdesivir | 1 | (3) | 7 | (16) | 0.07 |

ACE = angiotensin-converting enzyme; BMI = body mass index; COPD = chronic obstructive pulmonary disease; ECMO = extracorporeal membrane oxygenation; ICU = intensive care unit; IQR = interquartile range; SAPS II = Simplified Acute Physiology Score; SOFA = Sequential Organ Failure Assessment.
^{a}Baseline was defined as the index date (date of ICU admission).
^{b}Calculated using t test (mean age) or Wilcoxon test (all other variables).
^{c}N = 34.
dOne patient in each group was hospitalized before symptom onset; these patients contracted SARS-CoV-2 during a hospital stay and were subsequently transferred to the ICU.
^{e}SAPS II ranges from 0 (lowest risk of in-hospital mortality) to 163 (highest risk of in-hospital mortality).
^{f}N = 44.
^{g}SOFA scores consist of 6 organ systems, which are graded from 0 (no organ dysfunction) to 4 (high organ dysfunction); total scores range from 0 (no organ dysfunction; low risk of mortality) to 24 (high organ dysfunction; high risk of mortality).
^{h}With eculizumab, N = 28; without eculizumab, N = 42.

TABLE 30
Baseline^{a} Laboratory Values

| | With | Without | |
|---|---|---|---|
| Characteristic, | Eculizumab | Eculizumab | P |
| Mean (SD) | (n = 35) | (n = 45) | Value^{b} |
| D-dimers, ng/ml | n = 23 | n = 36 | |
| | 8909 (19,474) | 10,279 (28,276) | 0.69 |
| Prothrombin time, % ratio | n = 22 | n = 39 | |
| patient/control | | | |
| | 72.4 (11.8) | 71.8 (11.6) | 0.78 |
| Fibrinogens, g/L | n = 22 | n = 39 | |
| | 6.2 (1.6) | 6.6 (1.6) | 0.23 |
| Leukocytes, 10⁹/L | n = 23 | n = 40 | |
| | 8.3 (3.5) | 11.3 (14.2) | 0.65 |
| Platelets, 10⁹/L | n = 23 | n = 40 | |
| | 226.7 (109.4) | 227.6 (107.7) | 0.99 |
| Blood urea nitrogen, | n = 23 | n = 40 | |
| mmol/L | 5.6 (3.2) | 6.6 (4.4) | 0.36 |
| Creatinine, μmol/L | n = 23 | n = 40 | |
| | 71.0 (22.8) | 89.0 (60.3) | 0.48 |
| Total bilirubin, μmol/L | n = 22 | n = 40 | |
| | 8.8 (5.1) | 8.7 (5.2) | 0.92 |
| Conjugated bilirubin, | n = 23 | n = 39 | |
| μmol/L | 5.1 (3.5) | 5.0 (3.9) | 1 |
| AST, U/L | n = 23 | n = 40 | |
| | 71.3 (50.3) | 71.4 (44.7) | 0.79 |
| ALT, U/L | n = 23 | n = 40 | |
| | 55.2 (54.3) | 50.4 (36.8) | 0.59 |
| Troponin T, ng/L | n = 15 | n = 32 | |
| | 104.4 (211.2) | 109.3 (259.6) | 0.63 |
| CRP, mg/L | n = 23 | n = 40 | |
| | 168 (77) | 199 (99) | 0.23 |
| Procalcitonin, μg/L | n = 22 | n = 39 | |
| | 1.0 (1.8) | 6.2 (18.1) | 0.33 |
| Albumin, g/L | n = 21 | n = 37 | |
| | 24 (5) | 23 (5) | 0.90 |
| LDH, U/L | n = 21 | n = 40 | |
| | 435 (118) | 528 (588) | 0.97 |
| Lymphocytes, 10⁹/L | n = 22 | n = 40 | |
| | 0.8 (0.3) | 2.8 (12.8) | 0.63 |
| PaO2/FiO2, mmHg | n = 28 | n = 42 | |
| Mean (SD) | 119 (58) | 119 (64) | 0.90 |
| ARDS severity, n (%) | | | 0.81 |
| Severe (≤100) | 13 (46) | 22 (52) | |
| Mild/moderate (>100) | 15 (54) | 20 (48) | |
| Lactate, mmol/L | n = 28 | n = 42 | |
| | 1.3 (0.6) | 1.3 (0.7) | 0.66 |

ALT = alanine aminotransferase; AST = aspartate aminotransferase; CRP = C-reactive protein; LDH = lactate dehydrogenase; PaO2/FiO2 = ratio of partial pressure of arterial oxygen to fractional inspired oxygen.
^{a}Baseline was defined as the index date (date of ICU admission).
^{b}Calculated using Fisher's exact test (categorical variables) or Wilcoxon test (continuous variables).

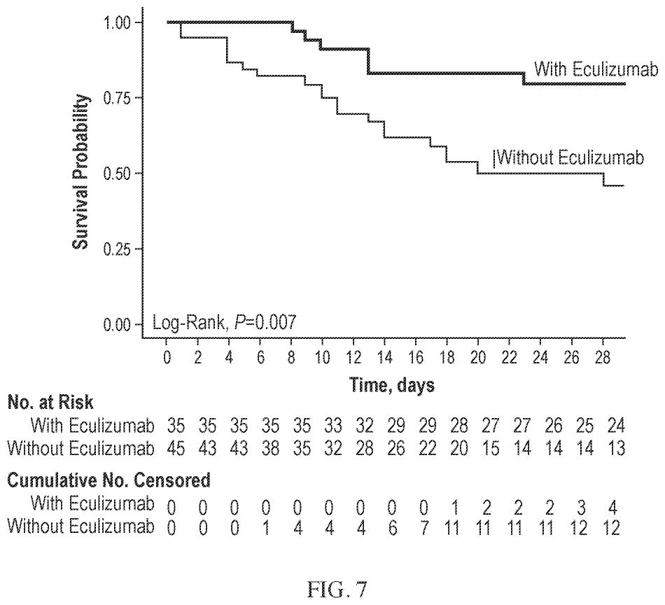

The estimated proportion of patients alive at day 15 was 82.9% (95% CI, 70.4%-95.3%) for patients treated with eculizumab and 62.1% (95% CI, 47.3%-76.9%) for patients treated without eculizumab; the estimated proportion of patients who were alive at day 28 was 79.8% (95% CI, 66.4%-93.2%) and 46.0% (95% CI, 29.4%-62.5%), respectively. In the prespecified statistical analysis, the log-rank test showed a significant difference in survival curves between groups (P=0.007; FIG. 7). In the 2 secondary analyses of the primary outcome, crude and sex- and SAPS II-adjusted hazard ratios (95% CI) for mortality at day 15 were 0.38 (0.15-0.97) and 0.19 (0.02-1.59), respectively (Table 31), and actual proportions for mortality at day 15 were 17.1% with eculizumab and 35.6% without eculizumab (P=0.08).

TABLE 31
Cox Proportional Hazard Model of the Association
of Mortality With Eculizumab

| | With | Without | | |
|---|---|---|---|---|
| Outcome, | Eculizumab | Eculizumab | Crude HR | Adjusted HR |
| n (%) | (n = 35) | (n = 45) | (95% CI)^{a} | (95% CI)^{b} |
| Mortality up | 6 (17) | 16 (36) | 0.38 | 0.19 |
| to day 15 | | | (0.15-0.97) | (0.02-1.59) |
| Mortality up | 7 (20) | 21 (47) | 0.32 | 0.29 |
| to day 28 | | | (0.14-0.77) | (0.06-1.42) |

^{a}Calculated using Cox proportional-hazard model with exposure as a time-dependent variable.
^{b}Calculated using Cox proportional-hazard model with exposure as a time-dependent variable and adjusted for sex and SAPS II.

In patients who were ventilated at baseline, mean (SD) number of days alive and free of mechanical ventilation at day 15 was 5.3 (4.9) with eculizumab and 2.3 (5.2) without eculizumab (P=0.1). The mean (SD) number of ICU-free days at day 28 was 13.2 (11.4) with eculizumab and 10.7 (10.9) without eculizumab.

At day 15, the proportion of patients treated with and without eculizumab who showed improvement in oxygenation, as measured by a shift in PaO2/FiO2 from ≤100 to >100 mmHg, was 18.2% and 14.3%, respectively (Table 32). Overtime, patients treated with eculizumab experienced more rapid clearance of lactate and more rapid increase in platelet count; other hematologic, chemistry, and respiratory parameters did not differ significantly between groups (Table 33). The proinflammatory cytokines IL-6, IL-17, and IFN-α2 decreased more rapidly over time in patients receiving eculizumab; there were no significant differences between groups in the evolution of other proinflammatory mediators or the anti-inflammatory mediators IL-4, IL-10, and IL-1RA (Table 33).

TABLE 32
Improvement in Oxygenation from Baseline to Day 15

| | With Eculizumab | | Without Eculizumab | |
|---|---|---|---|---|
| | (N = 35) | | (N = 45) | |
| | Baseline | Day 15 | Baseline | Day 15 |
| PaO2/FiO2, mmHg | (n = 28) | (n = 12) | (n = 42) | (n = 16) |
| ≤100, n (%) | 13 (46) | 4 (33) | 22 (52) | 10 (63) |
| >100, n (%) | 15 (54) | 8 (67) | 20 (48) | 6 (38) |
| Shift from ≤100 | — | 2/11 (18) | — | 2/14 (14) |
| to >100, n/N1 (%) | | | | |

N1 = number of patients with available data; PaO2/FiO2 = ratio of partial pressure of arterial oxygen to fractional inspired oxygen.

TABLE 33
Differences in Change in Hematologic, Chemistry, and Respiratory Parameters
and Inflammatory Markers Over Time

| | With Eculizumab | | Without Eculizumab | | β | P |
|---|---|---|---|---|---|---|
| Parameter | n | β^{a} | n | β^{a} | Difference | Value^{b} |
| Platelets, 10⁹/L | 29 | 14.8 | 42 | 0.5 | −14.3 | 0.002 |
| Prothrombin time, % ratio | 28 | −0.75 | 42 | −0.72 | 0.03 | 0.97 |
| patient/control | | | | | | |
| Fibrinogen, g/L | 28 | 0.01 | 42 | −0.09 | −0.10 | 0.23 |
| D-dimers, ng/ml | 29 | 1469.16 | 38 | 203.31 | −1265.85 | 0.21 |
| Creatinine, μmol/L | 29 | 0.26 | 42 | 3.80 | 3.54 | 0.42 |
| Blood urea nitrogen, mmol/L | 29 | 0.64 | 42 | 0.37 | −0.27 | 0.42 |
| Lymphocytes, 10⁹/L | 23 | −0.07 | 41 | −0.01 | 0.06 | 0.46 |
| Procalcitonin, μg/L | 29 | −0.03 | 41 | 0.41 | 0.44 | 0.69 |
| CRP, mg/L | 29 | 4.98 | 42 | −4.05 | −9.03 | 0.08 |
| Total bilirubin, μmol/L | 29 | 0.82 | 42 | 1.46 | 0.64 | 0.64 |
| Conjugated bilirubin, μmol/L | 29 | 0.52 | 41 | 1.16 | 0.64 | 0.53 |
| Troponin T, ng/L | 29 | −0.36 | 38 | −6.65 | −6.29 | 0.88 |
| PaO2/FiO2, mmHg | 29 | −3.53 | 42 | 2.37 | 5.90 | 0.56 |
| Lactate, mmol/L | 29 | −0.11 | 42 | −0.02 | 0.09 | <0.001 |
| TNF-α | 29 | −3.1 | 19 | −1.2 | 1.9 | 0.06 |
| IL-1β | 28 | −0.38 | 17 | −0.18 | 0.20 | 0.07 |
| IL-6 | 29 | −53.6 | 19 | −9.8 | 43.8 | 0.04 |
| IL-8 | 29 | −6.3 | 19 | 2.5 | 8.8 | 0.20 |
| IL-1RA | 29 | −87.1 | 19 | −21.7 | 65.4 | 0.60 |
| IL-4 | 29 | −0.05 | 19 | −0.03 | 0.02 | 0.30 |
| IL-10 | 29 | 7.4 | 19 | −0.1 | −7.5 | 0.60 |
| IL-17 | 29 | −0.3 | 19 | 0.1 | 0.4 | 0.01 |
| IFN-α2 | 29 | −0.2 | 19 | −0.07 | 0.13 | 0.03 |
| IFN-γ | 29 | −1.4 | 19 | 0.3 | 1.7 | 0.08 |

CRP = C-reactive protein; IFN = interferon; IL = interleukin; IL-1RA = IL-1 receptor antagonist;
PaO2/FiO2 = ratio of partial pressure of oxygen to fractional inspired oxygen; TNF = tumor necrosis factor.
^{a}Slope of change in parameter over time.
^{b}Calculated with linear mixed model for longitudinal data with time-by-group effect.

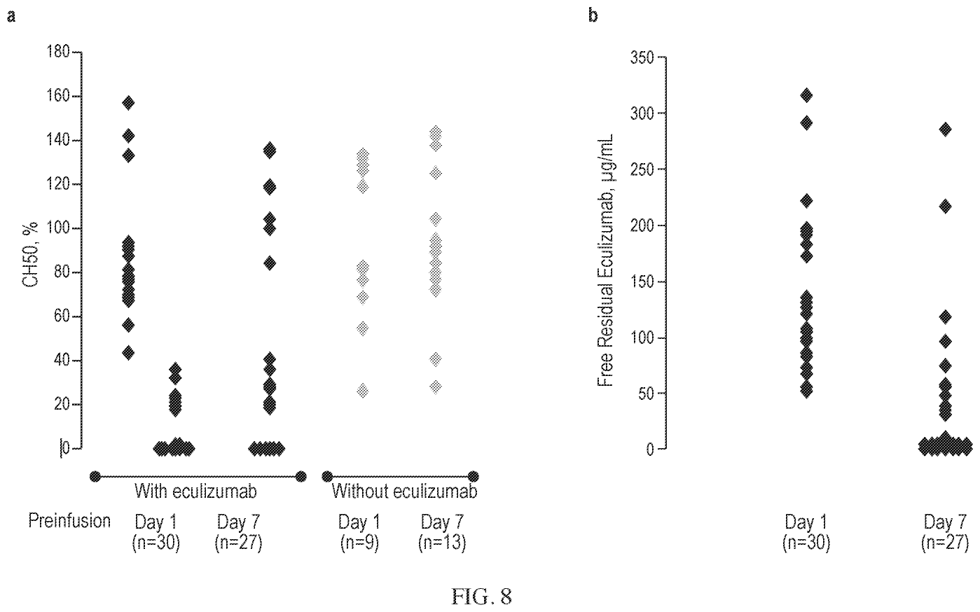

In patients receiving eculizumab, free residual eculizumab levels were variable (54-320 Dg/mL) on day 1 and undetectable in 15 of 27 patients on day 7 (FIG. B); CH50 activity was decreased on day 1, with detectable levels in 11 of 16 patients on day 7 (FIG. 8A). Serum soluble C5b-9 levels decreased over time (Table 35), whereas C3 and C4 levels remained stable (data not shown).

TABLE 35
Change in Complement C5b-9 Over Time

| | With | Without | P |
|---|---|---|---|
| Time | Eculizumab | Eculizumab | Value^{a} |
| Day 1 | n = 21 | n = 18 | |
| Mean (SD) | 264 (213) | 479 (452) | 0.09 |
| Median (range) | 198 (72-1025) | 336 (150-2000) | |
| Day 8 | n = 9 | n = 13 | |
| Mean (SD) | 334 (173) | 381 (203) | 0.60 |
| Median (range) | 296 (136-719) | 305 (140-925) | |
| Day 15 | n = 5 | n = 6 | |
| Mean (SD) | 185 (39) | 308 (88) | 0.03 |
| Median (range) | 180 (135-238) | 319 (178-396) | |

^{a}Calculated using Wilcoxon test.

Treatment-emergent SAEs of special interest are shown in Table 36. The proportion of patients experiencing a TESAE of an infectious complication was significantly greater with versus without eculizumab (57% vs 27%, respectively; P=0.01). Ventilator-associated pneumonia was reported in 51% versus 22% of patients treated with versus without eculizumab, respectively, bacteremia in 11% versus 2%, gastroduodenal hemorrhage in 14% versus 16%, and hemolysis in 3% versus 18%.

TABLE 36
Summary of TESAEs of Special Interest

| | With | Without | |
|---|---|---|---|
| | Eculizumab | Eculizumab | P |
| Patients With Event, n (%) | (n = 35) | (n = 45) | Value^{a} |
| Infectious complications | 20 (57) | 12 (27) | 0.01 |
| Ventilator-associated | 18 (51) | 11 (24) | 0.02 |
| pneumonia | | | |
| Bacteremia | 4 (11) | 1 (2) | 0.2 |
| Urinary tract infection | 0 | 1 (2) | 1 |
| Gastroduodenal hemorrhage | 5 (14) | 7 (16) | 1 |
| Hemolysis | 1 (3) | 8 (18) | 0.07 |
| Cardiac arrhythmia | 1 (3) | 1 (2) | 1 |
| Pulmonary embolism | 1 (3) | 0 | 1 |
| Catheter-related infection | 0 | 1 (2) | 1 |
| Cutaneous rash | 0 | 1 (2) | 1 |
| Leukopenia | 0 | 0 | — |

TESAE = treatment-emergent serious adverse event.
Adverse event terms are based on the Medical Dictionary for Regulatory Activities, version 22.1.
^{a}Calculated using Fisher's exact test.

5. Discussion

The analyses reported in the present study represent the largest and one of the first observations of the effectiveness of complement C5 inhibitor eculizumab as emergency treatment in patients with severe COVID-19. Compared with patients not receiving eculizumab but otherwise treated under the same institutional, national, and international guidelines, patients treated with eculizumab showed significantly improved survival (see, e.g., Alhazzani et al. (2020), Intensive Care Med 46:854-887). Improvements in key biomarkers suggest a potential mechanism of action involving improvements in oxygenation and inflammation subsequent to reduced terminal complement activation.

Serious respiratory manifestations contribute to high mortality in patients with severe COVID-19 (see, e.g., Gattinoni et al. (2020), Intensive Care Med:1-4); rates in patients who required mechanical ventilation range from 25% in a recent report from hospitals in the New York City region to 97% in a report from Wuhan, China (see, e.g., Richardson et al. (2020) JAMA 2020 May 26; 323(20):2052-2059), early in the pandemic (see, e.g., Zhou et al. (2020), Lancet 395:1054-1062). A randomized trial of lopinavir-ritonavir showed a nonsignificant reduction in mortality at day 28 from 25% with standard care to 19% with lopinavir-ritonavir (see, e.g., Cao B et al. (2020) N Engl J Med May 7; 382(19):1787-1799), and a small compassionate-use study of remdesivir showed a mortality rate of 13% (median follow-up, 18 days) (see, e.g., Grein et al. (2020) N Engl J Med June 11; 382(24):2327-2336); both studies included hospitalized patients with severe COVID-19, some of whom received mechanical ventilation. A phase 3 open-label trial of remdesivir, which excluded patients receiving ventilation at screening, showed overall day-14 mortality rates of 8% and 11% with 5- and 10-day treatment courses, respectively; in patients receiving invasive mechanical ventilation at day 5, rates were 40% and 17%, respectively (see, e.g., Goldman et al. (2020) N Engl J Med November 5; 383(19):1827-1837). In the study hospital, observed mortality rates 15 and 28 days after ICU admission were 36% and 47%, respectively, and these were reduced to 17% and 20% with the addition of eculizumab.

The present study represents the largest to date with respect to the number of patients treated with a complement inhibitor for severe COVID-19. In preclinical studies, inhibition of complement proteins C3, C5, or their downstream products reduced lung injury after infection with highly pathogenic viruses (see, e.g., Gralinski et al. (2018), mBio 9:e01753-01718; Sun et al. (2013), Am J Respir Cell Mol Biol 49:221-230; Jiang et al. (2018), Emerg Microbes Infect 7:77; and Sun et al. (2015), Clin. Infect Dis 60:586-595). Interestingly, inhibition of complement components C4 and factor B, which lie upstream from C3 and C5 in the complement pathway, appeared not to offer the same protection, indicating the importance of terminal complement blockade over inhibition of the alternative pathway (see, e.g., Gralinski et al. (2018) Complement activation contributes to severe acute respiratory syndrome coronavirus pathogenesis. mBio 9:e01753-01718). Taken together with clinical reports showing overactivation of the complement system (see, e.g., Pang et al. (2006), Clin Chem 52:421-429; Ohta et al. (2011), Microbiol Immunol 55:191-198; and Berdal et al. (2011), J Infect 63:308-316), these findings provide a rationale for blocking complement activation at C5 for improving survival in severe respiratory illness without compromising the immuno-protective and immunomodulatory functions served by other components of the complement pathway.

In the present study, baseline C5b-9 levels were elevated, and, at day 7, there was no detectable residual free eculizumab in some patients, suggesting possible overactivation of the complement pathway. The unexpected rapid clearance of eculizumab suggests that dosing may have been suboptimal and that higher or more frequent dosing may be appropriate in patients with severe COVID-19. However, the transient reduction in CH50 activity in patients who received eculizumab supports the concept that incomplete C5 inhibition is sufficient to enact clinical improvements. Reduced C5 activation represents an important mechanism for decreasing inflammation, cytokine production, and tissue damage, and biomarker analyses suggest that the clinical improvements in patients who received eculizumab may have been mediated by reduced inflammation and improved oxygenation (see, e.g., Wang et al. (2015), Emerg Microbes Infect 4:e28; and Keshari et al. (2017), Proceedings of the National Academy of Sciences 114:E6390-E6399). Concurrent with C5b-9 reduction, eculizumab-treated patients experienced reductions in the proinflammatory cytokines IL-6, IL-17, and IFN-α2, as well as accelerated improvements in platelet count and clearance of lactate, a robust biomarker of tissue hypoxia (see, e.g., Bakker et al. (2013),Annals of Intensive Care 3:12). Improvement in platelet count could be related to inhibition of complement-mediated thrombotic microangiopathy, a known effect of eculizumab in aHUS (see, e.g., Legendre et al. (2013), N Engl J Med 368:2169-2181). Based on preliminary evidence from a case series, overactivation of C5b-9 and subsequent complement-mediated microvascular injury could play an important role in severe COVID-19 (see, e.g., Magro et al. (2020) Transl Res June; 220:1-13). Collectively, these findings illustrate that C5 inhibition with eculizumab may lead to accelerated reduction of systemic and pulmonary inflammation induced by SARS-Cov-2, resulting in improved tissue oxygenation, improved survival, and more rapid resolution of severe illness.

Serious AEs are frequent in patients treated in the ICU. Patients in this study presented with severe pneumonia, acute lung injury, or ARDS. Reported TESAEs were generally consistent with SAEs typically seen in critically ill patients treated in the ICU (e.g., ventilator-associated pneumonia). Infectious complications were more commonly reported in patients treated with eculizumab. This could be related to prolonged survival, which may have exposed eculizumab-treated patients to additional risk of acquiring secondary infections. Overall, safety was consistent with the approximately 10 years of known safety data for eculizumab in complement-mediated diseases (see, e.g., Socie et al. (2019), Br J Haematol 185:297-310). Differences in TESAEs may represent spurious findings related to small sample size, and large randomized controlled studies are needed to characterize safety in patients with severe COVID-19.

Although this proof-of-concept study has some limitations, including the small number of patients, effort was made to make the analyses more robust by incorporating a controlled before-and-after design, using a prespecified primary outcome/analysis, and analyzing all measures recorded in the electronic health records to reduce bias. While there was no treatment randomization or blinding, patients were not selected for treatment; rather treatment was assigned based on availability of eculizumab at the time of ICU admission. There was no power analysis, and the study may have been underpowered to detect a significant difference in the secondary analyses. However, a significant difference was found for the prespecified analysis, suggesting these findings are worthy of follow-up. Some patients did not undergo all assessments, resulting in missing values. Although this work involved a single institution, which may limit generalizability, this helped to ensure consistency of care for all patients and highlights the importance of defining institutional and national guidelines. Nevertheless, there is potential for unidentified confounding variables, including nonspecific immune effects related to receipt of vaccines and antibiotics. Potential for such bias would exist even if treatment assignment had been fully consecutive.

In conclusion, in this single-institution proof-of-concept study, eculizumab treatment for patients with severe COVID-19 increased survival and accelerated the improvement of biomarkers of tissue hypoxia and inflammation. These preliminary findings underscore the efficacy and safety of the anti-C5 antibody eculizumab or a biosimilar antibody thereof in the treatment of patients with severe COVID-19.

### Example 11: Circulating sC5b9 Levels as Prognostic Indicator in Patients with COVID-19

Since the first cases were reported in December 2019, infection with the severe acute respiratory coronavirus 2 (SARS-CoV-2) commonly referred as COVID-19 has become a worldwide pandemic (see, e.g., Cucinotta D, Vanelli M., Acta Biomed 2020; 91:157-160). In patients with COVID-19 infection, respiratory deterioration has been associated not only to the increased viral loads in the lung but also to inadequate and exaggerated immune response (see, e.g., Risitano et al. Complement as a target in COVID-19? Nat Rev Immunol. 2020 June; 20(6):343-344)

Preclinical data have demonstrated a role for complement activation in CoV-mediated disease. Gralinski et al, found activation of the complement system in a mouse model of CoV-2 (see, e.g., Gralinski et al. mBio. 2018; 9(5)). In some patients with COVID-19, significant deposits of terminal complement components C5b-9 (membrane attack complex), C4d, and mannose binding lectin (MBL)-associated serine protease (MASP)2 have been found in the microvasculature of different organs, consistent with sustained, systemic activation of the lectin-complement pathway (see, e.g., Magro et al., Transl Res. 2020; S1931-5244(20)). MASP-2 mediated complement overactivation has also been reported in some patients by a Chinese group (see, e.g., Gao et al., Medrxiv. 2020).

However, there is little data on upregulation of terminal complement components C5b-9 (membrane attack complex), complement C4d, complement C3 and complement C4, in human patients suffering from severe COVID-19. More importantly, there is little scientific evidence, if any, that complement C5b9 can serve as biomarkers for monitoring or even predicting outcomes, i.e., time to discharge from hospital, in patients with severe COVID-19.

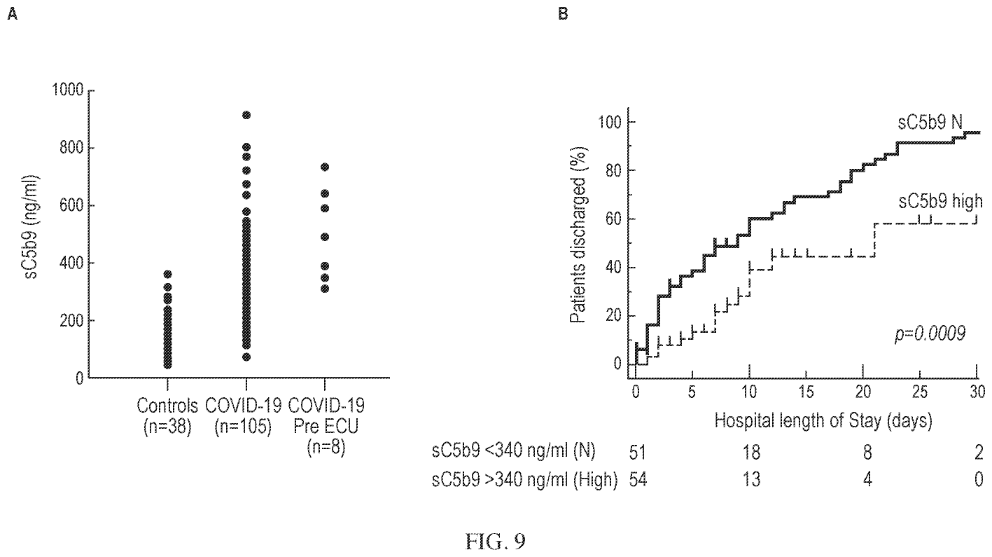

Complement activity was assessed in 113 patients with COVID-19 followed in Saint-Louis hospital (pneumology unit, infectious disease unit or ICU) using validated routine complement hemolytic activity (reported as CH50) by testing the capacity of patient plasma to lyse sheep erythrocytes coated with antibodies, C3, C4 and sC5b-9 circulating levels by nephelometry (Siemens) and ELISA (Quidel, San Diego, CA) respectively according to the instructions of the manufacturers. It was found that the levels of C3 and C4 were increased in 63.7% (72/113) and 35.5% (37/104) of patients, respectively. Moreover, the level of circulating sC5b-9 was increased in 54.8% of the COVID patients (62/113), highlighting the systemic C5 cleavage in COVID-19 (FIG. 9A). Furthermore, as shown in FIG. 9B, a significant correlation was found in our cohort of COVID patients between circulating sC5b-9 levels at time of sampling (high versus normal value) and the time to discharge from hospital (p=0.0009). Altogether, these data suggest that C5 activation contribute to disease severity.

Taken together, these findings suggest complement serves as a viable target for specific intervention in COVID-19 patients. In China, two deteriorating patients have been rescued using an anti-C5a monoclonal antibody (see, e.g., Gao et al., Medrxiv. 2020). In Italy, four patients with severe pneumoniae successfully recovered after treatment with Eculizumab (SOLIRIS; see, e.g., Diumo et al., European Review for Medical and Pharmacological Sciences. 2020; 4030-4037(24)). In the absence of proven effective therapy, a decision was taken to treat with Eculizumab COVID-19 patients with severe pneumonia on a compassionate-use basis. Five patients not requiring Intensive Care Unit (ICU) with severe pneumonia requiring ≥5 L/min of oxygen to maintain SpO2>97% and three patients with respiratory failure requiring mechanical ventilation and renal injury defined by AKI≥2 or requiring dialysis and vasopressive drugs support were treated. All patients had confirmed severe COVID-19 using specific RT-PCR (positive PCR on nasal swabs). Characteristics of the patients are detailed in Table 37. This report is based on data from patients who received Eculizumab during the period from Mar. 17, 2020, through Apr. 30, 2020.

TABLE 37
Patients Baseline Characteristics, at Time of Eculizumab Initiation and During Treatment

| | Patient #1 | Patient #2 | Patient #3 | Patient #4 | Patient #5 | Patient #6 | Patient #7 | Patient #8 |
|---|---|---|---|---|---|---|---|---|
| Age, years | 65 | 58 | 31 | 73 | 58 | 56 | 64 | 47 |
| Gender | M | F | M | M | M | F | F | M |
| Coexisting | Multiple | 7 months | High | High | High | High | High | None |
| conditions | Myeloma | post | Blood | Blood | Blood | Blood | Blood | |
| | | AlloBMT | Pressure | Pressure | Pressure | Pressure | Pressure | |
| | | (AML) | Takayasu | | Diabetes | Diabetes | Diabetes | |
| | | High Blood | disease | | Obesity | Obesity | Obesity | |
| | | Pressure | | | | | Asthma | |
| | | Obesity | | | | | | |
| | | Hypo- | | | | | | |
| | | thyroidism | | | | | | |

Baseline characteristics at admission

| | | | | | | | | |
|---|---|---|---|---|---|---|---|---|
| Symptoms | Fever, | Fever, | Dyspnea, | Fever, | Fever, | Fever, | Fever, | Fever, |
| | cough, | cough, | throat | cough, | cough | dyspnea | dyspnea | dyspnea |
| | dysgeusia, | dyspnea, | pain | dyspnea, | | | | |
| | anosmia | nausea, | | | | | | |
| | | vomiting | | | | | | |
| Time from first | 5 | 5 | 3 | 12 | 5 | 7 | 15 | 10 |
| symptoms (days) | | | | | | | | |
| Oxygen | 5 | 5 | 9 | 5 | 15 | 15 | 4 | 6 |
| supplementation to | | | | | | | | |
| maintain SpO2 | | | | | | | | |
| ≥97% | | | | | | | | |
| (liter/minute) | | | | | | | | |
| Creatinine | 24 | 49 | 115 | 79 | On | 20 | 40 | 109 |
| clearance (ml/mn) | | | | | dialysis | | | |
| C-reactive protein | 96 | 195 | 90 | 187 | 137 | 221 | NA | 95 |
| (mg/L) | | | | | | | | |

Characteristics at time of Eculizumab initiation

| | | | | | | | | |
|---|---|---|---|---|---|---|---|---|
| Time from first | 6 | 6 | 3 | 13 | 8 | 9 | 17 | 10 |
| symptoms to | | | | | | | | |
| Eculizumab first | | | | | | | | |
| injection (days) | | | | | | | | |
| Oxygen | 5 | 6 | 6 | 6 | Mechanical | Mechanical | Mechanical | High |
| supplementation to | | | | | ventilation | ventilation | ventilation | flow |
| maintain SpO2 | | | | | | | | |
| ≥97% | | | | | | | | |
| (liter/minute) | | | | | | | | |
| Additional | Enoxaparin | Enoxaparin | Enoxaparin | Enoxaparin | Unfractioned | Enoxaparin | Enoxaparin | Enoxaparin |
| treatments | 60 mg/day | 40 mg/day | 40 mg/day | 60 mg/day | heparin | 60 mg/day | 60 mg/day | 40 mg/day |
| | — | — | Dexa- | — | Dexa- | Dexa- | Dexa- | — |
| | | | methason^{ε} | | methason^{ε} | methason^{ε} | methason^{ε} | |

Evolution under Eculizumab treatment

| | | | | | | | | |
|---|---|---|---|---|---|---|---|---|
| Status at last | Alive | Alive | Alive | Alive | Death | Death | Alive | Alive |
| follow-up, location | Hospital | Hospital | d | Home | (MOF) | (PE) | Hospital | Home |
| and length of stay | 29 | 29 | 13 | 14 | 13 | 6 | 25 | 5 |
| in the hospital | | | | | | | | |
| (days) | | | | | | | | |
| Time from first | 28* | 28^{#} | 10 | 10 | NA | NA | 16 | 5 |
| injection to | | | | | | | | |
| ambient air of low | | | | | | | | |
| flow supplemental | | | | | | | | |
| oxygen (≤2 | | | | | | | | |
| liter/minute) (days) | | | | | | | | |
| Therapeutic | 900 mg | 900 mg | 900 mg | 1200 | 900 mg | 1200 mg | 1200 mg | 1200 |
| schedule and total | every 4 | every 4 | every 4 | mg | every 7 | every 4 | every 4 | mg |
| number of | days | days | days | every 4 | days | days | days | every 4 |
| Eculizumab | (4) | (5) | (3) | days | (2) | (1) | (4) | days |
| injection | | | | (3) | | | | (2) |

Abbreviations: AML: alloBMT: allogeneic Bone Marrow Transplantation; Acute Myeloid Leukemia; MOF: Mutli Organ Failure; PE: Pulmonary Embolism;
*diagnosed 10 days after Eculizumab injection with deep vein thrombosis and pulmonary embolism; ^{#}BMT related bronchiolitis flair 15 days after Eculizumab injection with a concomitant diagnosis of Parainfluenzae Virus Infection; ^{ε}Dexamethasone was given intravenously at dose of 20 mg once daily from day 1 to day 5, and then 10 mg once daily from day 6 to day 10.

The first ICU patient was treated according to SOLIRIS® SmPC—dosing regimen of atypical Hemolytic and Uremic Syndrome (aHUS, induction period with 900 mg every week). This patient has been monitored closely with regard to the complement activity during follow-up according to standard practice (see, e.g., Peffault de Latour et al., Blood. 2015; 775-83; 125(5)). The plasma of free Eculizumab was assessed using standard ELISA as previously described (see, e.g., Peffault de Latour et al., Blood. 2015; 775-83; 125(5)). The observation at day 7 showing a lack of complete inhibition of C5 with normal CH50 activity and undetectable free eculizumab circulating levels is suggestive of much higher clearance of eculizumab than usually seen after a single injection, probably related to the massive complement activation in those patients (FIG. 9A). Patients #2, 3 and 4 thus received 900 mg every 4 days allowing better but not optimal and prolonged complement blockade. Low eculizumab levels were observed at day 4 (below 50 μg/ml in 2 out the 3 patients) but an efficient complement blockage since day 1. These data point to a different eculizumab pharmacokinetics in COVID patients compared to patients afflicted with complement diseases like aHUS and paroxysmal nocturnal hemoglobinuria (PNH). The last 4 patients thus received 3 induction doses of 1200 mg at day 1, 4 and 8, which appears satisfactory from a PK/PD standpoint. Because of complement blockade the patients received prophylactic antibiotics against meningococcal infection prior to initiating SOLIRIS treatment and vaccination was done when possible (see, e.g., Diurno et al., European Review for Medical and Pharmacological Sciences. 2020; 4030-4037(24)).

At time of Eculizumab initiation, 3 patients were intubated, 1 received high flow oxygen and 4 were treated with standard oxygen support only. They all had elevated sC5b-9 circulating levels (FIG. 9A). Over a median follow-up of 18 days (from 5 to 29) after receiving the first dose of Eculizumab, 6 patients showed an improvement in the category of oxygen support, including 1 patient receiving mechanical ventilation who was extubated. By the date of the most recent follow-up, 3 patients had been discharged (day +5, +13 and +13 after first eculizumab injection, respectively), 3 patients are still hospitalized in non-ICU units at day +23, +28 and +28 after first eculizumab injection, respectively). Two patients who received invasive ventilation died (day +4 and +10 after first eculizumab injection, respectively). Patient #5 presented a septic shock and multi organ failure while patient #6 was diagnosed with massive pulmonary embolism and cardiac arrest. In addition, Patient #1 also presented severe thrombotic complications during evolution (deep venous thrombosis and pulmonary embolism).

Recent findings suggest that up to 30% of patients with severe COVID-19 infection develop life-threatening thrombotic complications (see, e.g., Klok et al., Thromb Res. 2020; S0049-3848(20)30120-1). In the present study, pulmonary embolism occurred despite complement blockade, in critically ill patients but also outside ICU, which confirm the higher thrombotic risk in those patients, including those under C5 therapy. Excessive inflammation, platelet activation, endothelial dysfunction, and stasis may predispose patients to thrombotic disease, both in the venous and arterial circulations (see, e.g., Bikdeli et al. J Am Coll Cardiol. 2020 Jun. 16; 75(23):2950-2973). Those considerations reinforce the recommendation to strictly apply thrombosis prophylaxis in COVID-19 patients, also in the context of complement inhibition (see, e.g., Connors J M and Levy J H. COVID-19 and Its Implications for Thrombosis and Anticoagulation. Blood. 2020 Jun. 4; 135(23):2033-2040).

Overall, the data in the present study show that terminal pathway of complement is overactivated in half the COVID-19 patients and is reflective of the severity of the disease. Complement suppression represents a common therapeutic approach in this setting. However, based on the observation that eculizumab pharmacokinetics in COVID patients differ significantly from reports in other complement mediated diseases, the present disclosure provides a novel therapeutic approach for treatment of COVID-19. Particularly, higher eculizumab doses and/or shorter intervals are applied to ensure efficient and sustained blockade of C5 activity. In patients enrolled in the present study, complement blockade did not prevent thrombotic occurrence, which may be attributed to sample size and heterogeneity in patient characteristics. Further outcome measurements are planned in a randomized, multicenter, prospective phase III clinical trial in severe non-ICU patients as well as in intubated patients (NCT ClinicalTrials.gov Identifier: NCT04346797). Patients receive a uniform schedule of Eculizumab (4 doses of 1200 mg every 3 days followed by 3 doses of 900 mg every 3 days) until oxygen support independence. More scientific evidence supporting use of eculizumab (SOLIRIS) in improving patient outcomes among severely ill COVID-19 patients is expected from this trial.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCARY
FFGSSPNWYFDVWGQGTLVTVSS
SEQ ID NO: 8
DIQMTQSPSSLSASVGDRVTITCGASENIYGALNWYQQKPGKAPKLLIYGA
TNLADGVPSRFSGSGSGTDFTLTISSLQPEDFATYYCQNVLNTPLTFGQGTK
VEIK
SEQ ID NO: 9
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
SEQ ID NO: 10
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPV
TKSFNRGEC
SEQ ID NO: 12
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSS ASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKC
CVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKV
SNKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVF
SCSVMHEALHNHYTQKSLSLSPGK
SEQ ID NO: 16
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMIS
RTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK

1. A method of treating a complement mediated disorder caused by a virus in a human subject comprising administering an effective amount of a polypeptide inhibitor of human complement C5 protein to the human subject,
wherein the virus is the coronavirus, Dengue virus (DENV), Ross River virus (RRV) and/or influenza virus (flu) and wherein the coronavirus is capable of causing lung or pulmonary injury in the subject.

2. The method of claim 1, wherein the coronavirus is selected from the group consisting of severe acute respiratory syndrome coronavirus (SARS-CoV), Middle East respiratory syndrome coronavirus (MERS-CoV), COVID-19 coronavirus (2019-nCoV), or a coronavirus related thereto.

3. (canceled)

4. The method of claim 1, comprising, prior to administering the effective amount of the polypeptide inhibitor of a complement C5 protein to the subject, determining that the subject infected with the coronavirus.

5. The method of claim 1, wherein the human subject exhibits at least one symptom or sign selected from (A) a respiratory symptom selected from: (1)) inflammation of cells in the large airway and parenchyma; (2) perivascular cuffing; (3) thickening of the interstitial membrane; (4) intra-alveolar edema; (5) rhinorrhea; (6) sneezing; (7) sore throat; (8) pneumonia; (9) ground-glass opacity; (10) RNAaemia; (11) acute respiratory distress syndrome (ARDS); and/or (B) a systemic disorder selected from (1) fever; (2) cough; (3) fatigue; (4) headache; (5) sputum production; (6) haemoptysis; (7) acute cardiac injury; (8) hypoxemia; (9) dyspnoea; (10) lymphopenia; (11) renal injury; and (12) diarrhea.

6. The method of claim 5, comprising the step of, prior to administering an effective amount of the polypeptide inhibitor of a complement C5 protein to the subject, determining that the subject's level of C5a is elevated or determining that the subject's serum level of lactate dehydrogenase (LDH) is elevated.

7. (canceled)

8. The method of claim 1, wherein the polypeptide inhibitor is a monoclonal antibody.

9. The method of claim 1, wherein the polypeptide inhibitor comprises a variable region of an antibody.

10. The method of claim 1, wherein the polypeptide inhibitor is eculizumab or an eculizumab variant, or antigen-binding fragment of eculizumab or eculizumab variant.

11. The method of claim 10, wherein the eculizumab or an eculizumab variant, or antigen-binding fragment of either is administered through intravenous infusion.

12. The method of claim 1, further comprising administering a second therapeutic agent to the subject.

13. The method of claim 12, wherein the subject experiences one or more of the following, after being administered the polypeptide inhibitor of a complement C5 protein: improved survival, decreased hemolysis, decreased disseminated intravascular coagulation, reduced complement levels, decreased levels of cytokines that are over-produced prior to the administration of the inhibitor, inhibition of pulmonary edema, maintained or improved lung functions, or reduced other symptoms of the disease.

14. The method of claim 1, wherein dosage level of the polypeptide inhibitor of a complement C5 protein to the subject is between about 1 mg per kg and about 100 mg per kg per subject per treatment.

15. (canceled)

16. The method of claim 1, wherein the subject receives a single unit dosage form of the polypeptide inhibitor of a complement C5 protein of 300 mg.

17. The method of claim 1, wherein the subject receives the polypeptide inhibitor of a complement C5 protein under the following treatment schedule: (i) about 900 mg of the polypeptide inhibitor every 7±2 days for the first 3 weeks, (ii) about 1200 mg of the polypeptide inhibitor for the 4th, 5th, and 6th dose on weeks 4, 6, and 8, and (iii) optionally about 1200 mg of the polypeptide inhibitor every other week for an additional 8 weeks.

18. The method of claim 1, wherein the subject experiences one or more of the following, after being administered the C5 inhibitor: improved chance for survival, reduced C5a level, reduced serum LDH level, little to no organ failure, decreased levels of one or more proinflammatory cytokines, improved one or more other symptoms of pulmonary edema, or combination thereof.

19. A method of treating a complement mediated disorder caused by a coronavirus in a human subject, comprising administering an effective amount of an anti-C5 antibody, or antigen binding fragment thereof, to the subject, wherein the complement mediated disorder is SARS, MERS, or COVID-19, wherein the method comprises an administration cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

20-24. (canceled)

25. The method of claim 19, wherein the treatment maintains a serum trough concentration of the anti-C5 antibody, or antigen binding fragment thereof, of 100 μg/ml or greater during the induction phase and/or the maintenance phase.

26-27. (canceled)

28. The method of claim 19, wherein the treatment results in terminal complement inhibition.

29-30. (canceled)

31. A method of treating a complement mediated disorder caused by a virus in a human subject, comprising intravenously administering a pharmaceutical composition comprising;
(i) eculizumab at a dose of 1200 mg on Days 1, 4, and 8; optionally administering 900 mg or 1200 mg of eculizumab at day 12 (D12) based on the therapeutic dose monitoring (TDM); administering 900 mg dose intravenously on day 15 (D15); optionally administering 900 mg or 1200 mg of intravenous eculizumab at day 18 (D18) based on TDM; and administering 900 mg dose intravenously on day 22 (D22), or
(ii) ravulizumab on Day 1 based on weight-based loading dose per United States Product Information (USPI) label for ULTOMIRIS® (ravulizumab-cwvz) injection, for intravenous use: administering 900 mg (or 600 mg for patients<60 kg) on day 5 (D5): administering 900 mg (or 600 mg for patients<60 kg) of ravulizumab on Day 10 (D10); and administering 900 mg of ravulizumab for all patients on Day 15 (D15);
wherein the virus is the coronavirus, Dengue virus (DENV), Ross River virus (RRV) and/or influenza virus (flu).

32. The method of claim 31, wherein the TDM comprises monitoring of a parameter selected from eculizumab plasma level and free C5 free C-5, and/or CH50 suppression, wherein, the optional dose is administered if the parameter is attenuated compared to a reference standard.

33-36. (canceled)

37. The method of claim 31, wherein the complement-mediated disorder caused by a virus is severe coronavirus disease 2019 (COVID-19) which comprises a need for hospitalization and/or treatment in an intensive care unit (ICU).

38. (canceled)

39. A method of effectively treating severe coronavirus disease-2019 (severe COVID-19) in a human patient with eculizumab, comprising
a. measuring a level of a marker which is C5b-9 (membrane attack complex; MAC) in the patient's blood sample, prior to and after treatment with eculizumab;
b. comparing the marker level to a reference standard;
c. titrating the treatment dose of eculizumab until the marker level in the human patient converges towards the reference standard; and
d. administering the titrated dose of eculizumab to the human patient.

40. The method of claim 39, wherein the marker is circulating sC5b9 level and the reference standard comprises a level of about 340 ng/ml, wherein the effective treatment comprises reduction in duration of hospitalization and/or length of intensive care unit (ICU) stay.

41-42. (canceled)


SEQUENCE LISTING (20 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK